(19) United States
(12) Patent Application Publication
(10) Pub. No.: US 2009/0030038 A1
(43) Pub. Date: Jan. 29, 2009

(54) Novel 8-Sulfonylamino-3 Aminosubstituted Chroman or Tetrahydronaphtalene Derivatives Modulating the 5Ht6 Receptor

(75) Inventors: Chester Chu, Loughborough (GB); Andrew Lister, Loughborough (GB); Gunnar Nordvall, Sodertalje (SE); Carl Petersson, Sodertalje (SE); Didier Rotticci, Sodertalje (SE); Daniel Sohn, Sodertalje (SE); Stefan Von Berg, Sodertalje (SE)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: ASTRAZENECA AB, Sodertalje (SE)

(21) Appl. No.: 11/915,173

(22) PCT Filed: May 22, 2006
(86) PCT No.: PCT/SE2006/000593

§ 371 Date: Jun. 30, 2008

(30) Foreign Application Priority Data
May 23, 2005 (SE) 0501166-3
May 23, 2005 (SE) 0501168-9

(51) Int. Cl. A61K 31/47; C07D 311/02; A61K 31/352; C07D 277/20; A61K 31/426; C07D 333/52; A61K 31/381; C07D 271/12; A61K 31/4245; C07D 319/16; A61K 31/357; C07D 405/02; A61K 31/4433; C07D 209/04; A61K 31/415; C07D 231/10; A61K 31/4164; C07D 261/02; A61P 3/00; A61P 25/00; A61K 31/18; C07C 311/15; A61K 31/42; C07D 263/54; A61K 31/423; C07D 215/00

(52) U.S. Cl. 514/312; 549/404; 514/456; 548/146; 514/365; 549/49; 514/443; 548/126; 514/364; 549/362; 514/452; 546/282.7; 514/337; 548/465; 514/414; 548/373.1; 514/406; 548/240; 514/378; 548/217; 514/375; 546/153; 564/84; 514/602

(57) ABSTRACT

The present invention relates to new compounds of formula I. (I) wherein R¹ to R¹², X, Q and n are as defined as in formula I, or salts, solvates or solvated salts thereof, processes for their preparation and to new intermediates used in the preparation thereof, pharmaceutical formulations containing said compounds and to the use of said compounds in therapy.

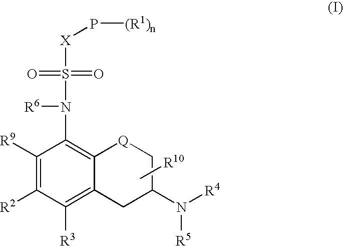

### FIELD OF THE INVENTION

The present invention relates to new compounds, to pharmaceutical formulations containing said compounds and to the use of said compounds in therapy. The present invention further relates to processes for the preparation of said compounds and to intermediates used in the preparation thereof.

### BACKGROUND OF THE INVENTION

Serotonin (5-hydroxy-tryptamine) (5-HT) receptors play an important role in many physiological and pathological functions like anxiety, sleep regulation, aggression, feeding and depression. The 5-HT receptors are distributed throughout the body and can be divided into seven different 5-HT receptor subtypes, i.e. 5-HT1-5-HT7, with different properties. The 5-HT6 receptor is mostly found in the central nervous system (CNS). From in situ hybridization studies it is known that the 5-HT6 receptor in rat brain is localized in areas like striatum, nucleus accumbens, olfactory tubercle and hippocampal formation (Ward et al., Neuroscience, 64, p 1105-1111, 1995).

Scientific research has revealed a potential therapeutic use for modulators of the 5-HT6 receptor, especially with regard to various CNS disorders. Blocking 5-HT6 receptor function has been shown to enhance cholinergic transmission (Bentley et al, Br J Pharmacol 126: 1537-1542, 1999; Riemer et al J Med Chem 46, 1273-1276). 5-HT6 antagonist have also been shown to reverse cognitive deficits in in vivo cognition models induced by the muscarinic antagonist scopolamine (Woolley et al. Phychopharmacolgy, 170, 358-367, 2003; Foley et al. Neuropsychopharmacology, 29 93-100, 2004)

Studies have shown that 5-HT6 antagonists increase levels of glutamate and aspartate in the frontal cortex and dorsal hippocampus as well as acetylcholine in the frontal cortex. These neurochemicals are known to be involved in memory and cognition (Dawson et al., Neuropsychopharmacology, 25(5), p 662-668, 2001) (Gerard et al., Brain Res., 746, p 207-219, 1997) (Riemer et al J Med Chem 46(7), p 1273-1276, 2003).

Acetylcholinesterase inhibitors increase the levels of acetylcholine in the CNS and are used in the treatment of cognitive disorders such as Alzheimer's disease. 5-HT6 antagonists may therefore be used in the treatment of cognitive disorders.

Studies have also shown that 5-HT6 antagonist increases the level of dopamine and noradrenaline in the medial prefrontal cortex (Lacroix et al. Synapse 51, 158-164, 2004). In addition, 5-HT6 receptor antagonists have been shown to improve performance in the attentional set shifting task (Hatcher et al. Psychopharmacology 181(2):253-9, 2005). Therefore, 5-HT6 ligands are expected to be useful in the treatment of disorders where cognitive deficits are a feature, such as schizophrenia. Several antidepressants and atypical antipsychotics bind to the 5-HT6 receptor and this may be a factor in their profile of activities (Roth et al., J. Pharm. Exp. Therapeut., 268, 1402-1420, 1994; Sleight et al., Exp. Opin. Ther. Patents, 8, 1217-1224, 1998; Kohen et al., J. Neurochem., 66(1), p 47-56, 1996; Sleight et al. Brit. J. Pharmacol., 124, p 556-562, 1998; Bourson et al., Brit. J. Pharmacol., 125, p 1562-1566, 1998).

Stean et al., (Brit. J. Pharmacol. 127 Proc. Supplement 131P, 1999) have described the potential use of 5-HT6 modulators in the treatment of epilepsy. 5-HT6 receptors have also been linked to generalized stress and anxiety states (Yoshioka et al., Life Sciences, 62, 17/18, p 1473-1477, 1998). 5-HT6 agonists have been shown to elevate levels of GABA in brain regions associated with anxiety and shown positive effects in models predictive of obsessive-compulsive disorder (Schechter et al. NeuroRx. 2005 October; 2(4): 590-611). The use of modulators for this receptor is therefore expected for a wide range of CNS disorders.

Pullagurla et al (Pharmacol Biochem Behav. 78(2):263-8, 2004) have described the potential use of 5-HT6 antagonists in disorders were the dopamine transmission is affected, for example a combination between a 5-HT6 antagonist and a dopamine enhancer for example levodopa/carbidopa or amantidine would be expected to have a advantages compared to a dopamine enhancer alone.

Moreover, a reduction in food intake in rats has been reported using 5-HT6 receptor modulators (Bentley et al., Br. J. Pharmacol. Suppl. 126, P66, 1999; Bentley et al. J. Psychopharmacol. Supl. A64, 255, 1997; Pendharkar et al Society for Neuroscience, 2005). 5-HT6 receptor modulators may therefore also be useful in the treatment of feeding disorders like anorexia, obesity, bulimia and similar disorders and also type 2 diabetes.

### DETAILED DESCRIPTION OF THE INVENTION

The object of the present invention is to provide compounds exhibiting a modulating activity at the 5-hydroxy-tryptamine 6 receptor.

The present invention provides compounds of formula I

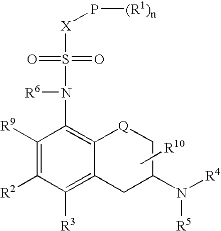

wherein:
P is C₆₋₁₀arylC₀₋₆alkyl, C₅₋₁₁heteroarylC₀₋₆alkyl, C₃₋₇cycloalkylC₀₋₆alkyl, C₃₋₇heterocycloalkylC₀₋₆alkyl or C₂₋₁₀alkyl;
R¹ is hydrogen, hydroxy, halogen, C₁₋₁₀alkyl, C₂₋₁₀alkenyl, C₂₋₁₀alkynyl, C₁₋₁₀alkoxy, N(R¹¹)₂, C₆₋₁₀arylC₀₋₆alkyl, C₅₋₁₁heteroarylC₀₋₆alkyl, C₁₋₆haloalkyl, C₁₋₆haloalkylO, R⁷OC₀₋₆alkyl, cyano, NO₂, SR⁷, R⁷SO₂C₀₋₄alkyl, SOR⁷, R⁷CON(R⁸)C₀₋₄alkyl, N(R⁸)SO₂R⁷, COR⁷, COOR⁸, OSO₂R⁷, (R⁸)₂NCOC₀₋₆alkyl, oxo or SO₂N(R⁹)₂;

n is 0, 1, 2, 3, 4 or 5;

X is a single bond, C₁₋₃alkyl or NR⁶, or X is N in a heteroalkyl or C₅₋₁₁heteroaryl; or

N, SO₂, X and P form together a C₈₋₁₁heteroaryl or C₈₋₁₁bicycloheteroalkyl;

### Q is CH or O;

R² is hydrogen, hydroxy, halogen, C₁₋₁₀alkyl, C₂₋₁₀alkenyl, C₂₋₁₀alkynyl, C₁₋₁₀alkoxy, N(R¹¹)₂, C₆₋₁₀arylC₀₋₆alkyl, C₅₋₆heteroarylC₀₋₆alkyl, C₁₋₆haloalkyl, C₁₋₆haloalkylO, R⁷OC₀₋₆alkyl, cyano, SR⁷, SO₂R⁸, SOR⁷, NCOR⁷, NR⁸SO₂R⁷, COR⁷, COOR⁷, OSO₂R⁷, CON(R⁸)₂ or SO₂N(R⁸)₂;
R³ is hydrogen, hydroxy, halogen, C₁₋₁₀alkyl, C₂₋₁₀alkenyl, C₂₋₁₀alkynyl, C₁₋₁₀alkoxy, N(R¹¹)₂, C₆₋₁₀arylC₀₋₆alkyl, C₅₋₆heteroarylC₀₋₆alkyl, C₁₋₆haloalkyl, C₁₋₆haloalkylO, R⁷OC₀₋₆alkyl, cyano, SR⁷, SO₂R⁷, SOR⁷, N(R⁸)COR⁷, N(R⁸SO₂R⁷, COR⁷, COOR⁷, OSO₂R⁷, CON(R⁸)₂ or SO₂N(R⁸)₂;
R⁴ and R⁵ are selected independently from hydrogen, C₁₋₅alkyl, C₁₋₅haloalkyl, C₂₋₅alkenyl, C₂₋₅alkynyl, C₃₋₆cycloalkyl, C₅₋₆arylC₁₋₂alkyl and C₅₋₆heteroarylC₁₋₂alkyl and may be substituted by one or more groups selected independently from halogen, hydroxyl, cyano and C₁₋₅alkoxy, or
R⁴ and R⁵ form together C₃₋₇heterocycloalkyl, whereby R⁴ and R⁵ may be substituted by one or more groups selected independently from hydrogen, halogen, C₁₋₆alkyl, C₁₋₆haloalkyl, C₅₋₆aryl, C₅₋₆heteroaryl, COR¹², SO₂R¹², OR¹², cyano, SO₂N(R¹¹)₂ and oxo substituted on β or γ position;
R⁶ is hydrogen, C₁₋₆alkyl, C₃₋₆cycloakyl, R⁷OC₁₋₆alkyl, C₁₋₆haloalkyl, C₁₋₆cyanoalkyl, (R¹¹)₂NCOC₀₋₆alkyl or R¹²SO₂C₁₋₆alkyl;
R⁷ is C₁₋₁₀alkyl, C₁₋₆haloalkyl, C₆₋₁₀arylC₀₋₆alkyl, C₅₋₆heteroarylC₀₋₆alkyl, C₃₋₇cycloalkylC₀₋₆alkyl or C₁₋₆alkoxyC₆₋₁₀aryl;
R⁵ is a hydrogen, C₁₋₁₀alkyl, C₃₋₇cycloalkylC₀₋₆alkyl, C₆₋₁₀arylC₀₋₆alkyl, C₁₋₆haloalkyl or C₅₋₆heteroarylC₀₋₆alkyl, or
R⁷ and R⁸ form together a C₅₋₆heteroaryl or C₃₋₇heterocycloalkyl; and whereby any aryl and heteroaryl under R¹, R⁷ and R⁸ may be substituted by one or more groups selected independently from hydrogen, halogen, hydroxy, C₁₋₆haloalkyl, cyano, alkyl, OR¹², oxo, C₁₋₅alkoxy, SOR¹², SR¹¹, CON(R¹¹)₂, N(R¹¹)COR¹², SO₂R¹², N(R¹¹)₂ and COR¹²;
R⁹ is hydrogen, halogen, hydroxy, C₁₋₆alkoxy, C₁₋₆haloalkoxy, C₁₋₆haloalkyl, C₁₋₆alkyl or COR¹²;
R¹⁰ is hydrogen, C₁₋₆alkyl, C₁₋₆alkoxy or C₁₋₆haloalkyl;
R¹¹ is hydrogen, C₁₋₆alkyl or C₁₋₆haloalkyl; and
R¹² is C₁₋₆alkyl or C₁₋₆haloalkyl, or
R¹¹ and R¹² form together a C₃₋₇cycloalkyl or C₃₋₇heterocycloalkyl, whereby R¹¹ and R¹² may be substituted by one or more groups selected independently from hydrogen, halogen, hydroxy, cyano, C₁₋₃alkyl, C₁₋₃alkoxy and C₁₋₃haloalkyl, or salts, solvates or solvated salts thereof.

Another embodiment of the invention relates to compounds of formula I wherein: wherein:

P is C₆₋₁₀arylC₀₋₆alkyl, C₅₋₁₁heteroarylC₀₋₆alkyl, C₃₋₇cycloalkylC₀₋₆alkyl or C₂₋₁₀alkyl;
R¹ is hydrogen, hydroxy, halogen, C₁₋₁₀alkyl, C₁₋₁₀alkoxy, C₆₋₁₀arylC₀₋₆alkyl, C₅₋₁₁heteroarylC₀₋₆alkyl, C₁₋₆haloalkyl, R⁷OC₀₋₆alkyl, NO₂, R⁷SO₂C₀₋₄alkyl, R⁷CON(R⁸)C₀₋₄alkyl, COR⁷ or SO₂N(R⁸)₂;
n is 0, 1, 2, 3 or 4;
X is a single bond or NR⁶;

### Q is CH or O;

R² is hydrogen;
R³ is halogen or C₁₋₁₀alkoxy;
R⁴ and R⁵ are selected independently from hydrogen or C₁₋₅alkyl, or
R⁴ and R⁵ form together C₃₋₇heterocycloalkyl;
R⁶ is R⁶ is hydrogen;
R⁷ is C₁₋₁₀alkyl, C₁₋₆haloalkyl, C₆₋₁₀arylC₀₋₆alkyl, C₃₋₇cycloalkylC₀₋₆alkyl or C₁₋₆alkoxyC₆₋₁₀aryl;
R⁸ is a hydrogen, C₁₋₁₀alkyl, C₆₋₁₀arylC₀₋₆alkyl or C₁₋₆haloalkyl; and whereby any aryl and heteroaryl under R¹, R⁷ and R⁸ may be substituted by one or more groups selected independently from hydrogen, halogen, C₁₋₆haloalkyl, cyano, C₁₋₅alkoxy or SR¹¹;
R⁹ is hydrogen; and
R¹⁰ is hydrogen;
or salts, solvates or solvated salts thereof.

In a further embodiment of the invention P is phenyl, naftyl or tetralinyl.

In yet another embodiment of the invention P is pyridinyl, pyrrolyl, benzodioxanyl, methylpyridinyl, benzofuryl, thiophenyl, thioimidazolyl, benzothiaimidazolyl, benzofurazanyl, thiazolylpyrazolyl, imidazolyl, methylphenyl, indolinyl, benzopyrrolidinyl, quinoline, isoquinoline, thiazolyl, imidazothiazolyl, furyl, ethyl, cyclopropyl, thienyl or ethylnaphtyl.

In one embodiment P is chromane or indane.

In another embodiment of the invention P is substituted with 0, 1, 2, 3 or 4 groups R¹, wherein the number of R¹ substituents is designated by the term n. In another embodiment of the invention n is 0, 1, 2 or 3.

Where P is substituted by more than one R¹ group it is to be understood that the R¹ substituent may be the same or different.

In a further embodiment of the invention R¹ is hydrogen, chloro, fluoro, bromo, iodo, methyl, ethyl, i-propyl, n-propyl, n-butyl, tert-butyl, phenoxy, methoxy, ethoxy, propoxy, pyridinyl, isooxazole, benzooxazolyl, thiophenyl, methylCON, phenylNCOmethyl, phenylSO₂-ethyl, nitro, phenylSO₂, methylSO₂, NH₂SO₂, phenyl, cyano, COOmethyl, pyrimidyl, pyrazolyl, COmethyl or hydroxy.

In another embodiment R¹ is C₁₋₆haloalkyl, C₁₋₆haloalkylO or NCOhalomethyl. In yet a another embodiment R¹ is fluoromethyl, difluoromethyl, trifluoromethyl, fluoromethoxy, difluoromethoxy or trifluoromethoxy.

In one embodiment of the invention R³ is halogen, methoxy, ethoxy or propoxy. In another embodiment R³ is C₁₋₆haloalkyl or C₁₋₆haloalkylO. In yet another embodiment R³ is fluoromethyl, difluoromethyl, trifluoromethyl, fluoromethoxy, difluoromethoxy or trifluoromethoxy.

In a further embodiment X is a bond. In another embodiment X is NH. In yet a further embodiment X is N in a mono or bicyclic C₅₋₁₁heteroalkyl or C₈₋₁₂heteroaryl. In one embodiment X is N in an indol, indoline, tetrahydroquinoline, tetrahydroisoquinoline, benzoxazepine, isoindoline or benzazepine.

In one embodiment of the invention R⁴ and R⁵ are selected independently from C₁₋₃alkyl, and C₁₋₃haloalkyl. In another embodiment R⁴ and R⁵ are selected independently from hydrogen, methyl, ethyl, i-propyl, n-propyl and fluoroethyl.

In a further embodiment R⁴ and R⁵ form together C₃₋₇heterocycloalkyl ring. In yet a further embodiment R⁴ and R⁵ form together a pyrrolidine.

In another embodiment R⁴ and R⁵ form together morpholine, aminolactam optionally substituted on the lactam nitrogen or N-substituted piperazine whereby the substituent on the piperazine nitrogen may be selected independently from hydrogen, C₁₋₆alkyl, C₅₋₆aryl, C₅₋₆heteroaryl, COR⁷, SO₂R⁷ and SO₂N(R⁸)R⁶.

Another embodiment of the invention relates to compounds selected from the group consisting of
(3R)-5-Methoxy-N,N-dimethyl-8-[(phenylsulfonyl)amino]chroman-3-ammonium acetate,
(3R)-8-{[(4-Chlorophenyl)sulfonyl]amino}-5-methoxy-N,N-dimethylchroman-3-ammonium acetate,
3-Bromo-N-[(3R)-3-(dimethylamino)-5-methoxy-3,4-dihydro-2H-chromen-8-yl]benzenesulfonamide,
N-[(3R)-3-(Dimethylamino)-5-methoxy-3,4-dihydro-2H-chromen-8-yl]biphenyl-4-sulfonamide,
N-[(3R)-3-(dimethylamino)-5-methoxy-3,4-dihydro-2H-chromen-8-yl]-2-methoxy-4-methylbenzenesulfonamide,
6-chloro-N-[(3R)-3-(dimethylamino)-5-methoxy-3,4-dihydro-2H-chromen-8-yl]imidazo[2,1-b][1,3]thiazole-5-sulfonamide,
N-[(3R)-3-(dimethylamino)-5-methoxy-3,4-dihydro-2H-chromen-8-yl]-2-(methylsulfonyl)benzenesulfonamide,
5-chloro-N-[(3R)-3-(dimethylamino)-5-methoxy-3,4-dihydro-2H-chromen-8-yl]-3-methyl-1-benzothiophene-2-sulfonamide,
7-chloro-N-[(3R)-3-(dimethylamino)-5-methoxy-3,4-dihydro-2H-chromen-8-yl]-2,1,3-benzoxadiazole-4-sulfonamide,
N-[(3R)-3-(dimethylamino)-5-methoxy-3,4-dihydro-2H-chromen-8-yl]-3-(trifluoromethoxy)benzenesulfonamide,
N-[(3R)-3-(dimethylamino)-5-methoxy-3,4-dihydro-2H-chromen-8-yl]-2,3-dihydro-1,4-benzodioxine-6-sulfonamide,
3-(2-chlorophenoxy)-N-[(3R)-3-(dimethylamino)-5-methoxy-3,4-dihydro-2H-chromen-8-yl]benzenesulfonamide,
4,5-dichloro-N-[(3R)-3-(dimethylamino)-5-methoxy-3,4-dihydro-2H-chromen-8-yl]thiophene-2-sulfonamide,
N-[(3R)-3-(dimethylamino)-5-methoxy-3,4-dihydro-2H-chromen-8-yl]-2-(1-naphthyl)ethanesulfonamide,
4-chloro-N-[(3R)-3-(dimethylamino)-5-methoxy-3,4-dihydro-2H-chromen-8-yl]naphthalene-1-sulfonamide,
4′-cyano-N-[(3R)-3-(dimethylamino)-5-methoxy-3,4-dihydro-2H-chromen-8-yl]-1,1′-biphenyl-2-sulfonamide,
N-[(3R)-3-(dimethylamino)-5-methoxy-3,4-dihydro-2H-chromen-8-yl]-3-(trifluoromethyl)benzenesulfonamide,
N-[(3R)-3-(dimethylamino)-5-methoxy-3,4-dihydro-2H-chromen-8-yl]-5-pyridin-2-ylthiophene-2-sulfonamide,
N-[3-({[(3R)-3-(dimethylamino)-5-methoxy-3,4-dihydro-2H-chromen-8-yl]amino}sulfonyl)phenyl]acetamide,
1-acetyl-5-bromo-N-[(3R)-3-(dimethylamino)-5-methoxy-3,4-dihydro-2H-chromen-8-yl]indoline-6-sulfonamide,
4-cyano-N-[(3R)-3-(dimethylamino)-5-methoxy-3,4-dihydro-2H-chromen-8-yl]benzenesulfonamide,
N-[(3R)-3-(dimethylamino)-5-methoxy-3,4-dihydro-2H-chromen-8-yl]-4-propylbenzenesulfonamide,
N-[(3R)-3-(dimethylamino)-5-methoxy-3,4-dihydro-2H-chromen-8-yl]naphthalene-2-sulfonamide,
N-[(3R)-3-(dimethylamino)-5-methoxy-3,4-dihydro-2H-chromen-8-yl]-4-methylbenzenesulfonamide,
4-bromo-N-[(3R)-3-(dimethylamino)-5-methoxy-3,4-dihydro-2H-chromen-8-yl]benzenesulfonamide,
3-bromo-5-chloro-N-[(3R)-3-(dimethylamino)-5-methoxy-3,4-dihydro-2H-chromen-8-yl]thiophene-2-sulfonamide,
4-tert-butyl-N-[(3R)-3-(dimethylamino)-5-methoxy-3,4-dihydro-2H-chromen-8-yl]benzenesulfonamide,
N-[(3R)-3-(dimethylamino)-5-methoxy-3,4-dihydro-2H-chromen-8-yl]-4-methoxybenzenesulfonamide,
2-chloro-N-[(3R)-3-(dimethylamino)-5-methoxy-3,4-dihydro-2H-chromen-8-yl]benzenesulfonamide,
N-[4-({[(3R)-3-(dimethylamino)-5-methoxy-3,4-dihydro-2H-chromen-8-yl]amino}sulfonyl)phenyl]acetamide,
2-bromo-N-[(3R)-3-(dimethylamino)-5-methoxy-3,4-dihydro-2H-chromen-8-yl]benzenesulfonamide,
N-{[5-({[(3R)-3-(dimethylamino)-5-methoxy-3,4-dihydro-2H-chromen-8-yl]amino}sulfonyl)thien-2-yl]methyl}benzamide,
N-[(3R)-3-(dimethylamino)-5-methoxy-3,4-dihydro-2H-chromen-8-yl]-4-(trifluoromethyl)benzenesulfonamide,
N-[(3R)-3-(dimethylamino)-5-methoxy-3,4-dihydro-2H-chromen-8-yl]-4-ethylbenzenesulfonamide,
N-[(3R)-3-(dimethylamino)-5-methoxy-3,4-dihydro-2H-chromen-8-yl]-2-nitrobenzenesulfonamide,
2-chloro-N-[(3R)-3-(dimethylamino)-5-methoxy-3,4-dihydro-2H-chromen-8-yl]-5-(trifluoromethyl)benzenesulfonamide,
N-[(3R)-3-(dimethylamino)-5-methoxy-3,4-dihydro-2H-chromen-8-yl]-4-methyl-3-nitrobenzenesulfonamide,
N-[(3R)-3-(dimethylamino)-5-methoxy-3,4-dihydro-2H-chromen-8-yl]naphthalene-1-sulfonamide,
4-chloro-N-[(3R)-3-(dimethylamino)-5-methoxy-3,4-dihydro-2H-chromen-8-yl]-3-nitrobenzenesulfonamide,
4-chloro-N-[(3R)-3-(dimethylamino)-5-methoxy-3,4-dihydro-2H-chromen-8-yl]benzenesulfonamide,
2,4-dichloro-N-[(3R)-3-(dimethylamino)-5-methoxy-3,4-dihydro-2H-chromen-8-yl]benzenesulfonamide,
N-[5-({[(3R)-3-(dimethylamino)-5-methoxy-3,4-dihydro-2H-chromen-8-yl]amino}sulfonyl)-4-methyl-1,3-thiazol-2-yl]acetamide,
N-[(3R)-3-(dimethylamino)-5-methoxy-3,4-dihydro-2H-chromen-8-yl]thiophene-2-sulfonamide,
N-[(3R)-3-(dimethylamino)-5-methoxy-3,4-dihydro-2H-chromen-8-yl]-4-nitrobenzenesulfonamide,
3,5-dichloro-N-[(3R)-3-(dimethylamino)-5-methoxy-3,4-dihydro-2H-chromen-8-yl]-2-hydroxybenzenesulfonamide,
N-[(3R)-3-(dimethylamino)-5-methoxy-3,4-dihydro-2H-chromen-8-yl]-3-nitrobenzenesulfonamide,
N-[(3R)-3-(dimethylamino)-5-methoxy-3,4-dihydro-2H-chromen-8-yl]-2,5-dimethoxybenzenesulfonamide,
4,5-dibromo-N-[(3R)-3-(dimethylamino)-5-methoxy-3,4-dihydro-2H-chromen-8-yl]thiophene-2-sulfonamide,
5-bromo-N-[(3R)-3-(dimethylamino)-5-methoxy-3,4-dihydro-2H-chromen-8-yl]-2-methoxybenzenesulfonamide,
N-[(3R)-3-(dimethylamino)-5-methoxy-3,4-dihydro-2H-chromen-8-yl]-5-(phenylsulfonyl)thiophene-2-sulfonamide,
N-[(3R)-3-(dimethylamino)-5-methoxy-3,4-dihydro-2H-chromen-8-yl]-5-[1-methyl-5-(trifluoromethyl)-1H-pyrazol-3-yl]thiophene-2-sulfonamide,
2-cyano-N-[(3R)-3-(dimethylamino)-5-methoxy-3,4-dihydro-2H-chromen-8-yl]benzenesulfonamide,
5-chloro-N-[(3R)-3-(dimethylamino)-5-methoxy-3,4-dihydro-2H-chromen-8-yl]-1,3-dimethyl-1H-pyrazole-4-sulfonamide,
N-[(3R)-3-(dimethylamino)-5-methoxy-3,4-dihydro-2H-chromen-8-yl]benzenesulfonamide,
N-[(3R)-3-(dimethylamino)-5-methoxy-3,4-dihydro-2H-chromen-8-yl]-3,5-dimethylisoxazole-4-sulfonamide,
N-[(3R)-3-(dimethylamino)-5-methoxy-3,4-dihydro-2H-chromen-8-yl]-1-methyl-1H-imidazole-4-sulfonamide,
N-[(3R)-3-(dimethylamino)-5-methoxy-3,4-dihydro-2H-chromen-8-yl]-5-isoxazol-3-ylthiophene-2-sulfonamide,
methyl 3-({[(3R)-3-(dimethylamino)-5-methoxy-3,4-dihydro-2H-chromen-8-yl]amino}sulfonyl)thiophene-2-carboxylate,
N-[(3R)-3-(dimethylamino)-5-methoxy-3,4-dihydro-2H-chromen-8-yl]-4-phenoxybenzenesulfonamide,
N-[(3R)-3-(dimethylamino)-5-methoxy-3,4-dihydro-2H-chromen-8-yl]-3,5-bis(trifluoromethyl)benzenesulfonamide,
2,6-dichloro-N-[(3R)-3-(dimethylamino)-5-methoxy-3,4-dihydro-2H-chromen-8-yl]benzenesulfonamide,
N-[(3R)-3-(dimethylamino)-5-methoxy-3,4-dihydro-2H-chromen-8-yl]-2,6-difluorobenzenesulfonamide,
N-[(3R)-3-(dimethylamino)-5-methoxy-3,4-dihydro-2H-chromen-8-yl]-2-methyl-5-nitrobenzenesulfonamide,
N-[(3R)-3-(dimethylamino)-5-methoxy-3,4-dihydro-2H-chromen-8-yl]-4-tertpentylbenzenesulfonamide,
N-[(3R)-3-(dimethylamino)-5-methoxy-3,4-dihydro-2H-chromen-8-yl]-3,4,5-trimethoxybenzenesulfonamide,
N-[(3R)-3-(dimethylamino)-5-methoxy-3,4-dihydro-2H-chromen-8-yl]-3-methylbenzenesulfonamide,
N-[(3R)-3-(dimethylamino)-5-methoxy-3,4-dihydro-2H-chromen-8-yl]-2-(trifluoromethoxy)benzenesulfonamide,
4-bromo-N-[(3R)-3-(dimethylamino)-5-methoxy-3,4-dihydro-2H-chromen-8-yl]-2-fluorobenzenesulfonamide,
N-[(3R)-3-(dimethylamino)-5-methoxy-3,4-dihydro-2H-chromen-8-yl]-2-methyl-4-nitrobenzenesulfonamide,
3-chloro-N-[(3R)-3-(dimethylamino)-5-methoxy-3,4-dihydro-2H-chromen-8-yl]benzenesulfonamide,
2-chloro-N-[(3R)-3-(dimethylamino)-5-methoxy-3,4-dihydro-2H-chromen-8-yl]-4-fluorobenzenesulfonamide,
3-chloro-N-[(3R)-3-(dimethylamino)-5-methoxy-3,4-dihydro-2H-chromen-8-yl]-4-fluorobenzenesulfonamide,
2-chloro-N-[(3R)-3-(dimethylamino)-5-methoxy-3,4-dihydro-2H-chromen-8-yl]-4-(trifluoromethyl)benzenesulfonamide,
N-[(3R)-3-(dimethylamino)-5-methoxy-3,4-dihydro-2H-chromen-8-yl]-1-phenylmethanesulfonamide,
N-[(3R)-3-(dimethylamino)-5-methoxy-3,4-dihydro-2H-chromen-8-yl]-2,4-difluorobenzenesulfonamide,
N-[(3R)-3-(dimethylamino)-5-methoxy-3,4-dihydro-2H-chromen-8-yl]-5-fluoro-2-methylbenzenesulfonamide,
N-[(3R)-3-(dimethylamino)-5-methoxy-3,4-dihydro-2H-chromen-8-yl]-4-(trifluoromethoxy)benzenesulfonamide,
2,5-dichloro-N-[(3R)-3-(dimethylamino)-5-methoxy-3,4-dihydro-2H-chromen-8-yl]benzenesulfonamide,
2,4,6-trichloro-N-[(3R)-3-(dimethylamino)-5-methoxy-3,4-dihydro-2H-chromen-8-yl]benzenesulfonamide,
3-bromo-N-[(3R)-3-(dimethylamino)-5-methoxy-3,4-dihydro-2H-chromen-8-yl]benzenesulfonamide,
3-chloro-N-[(3R)-3-(dimethylamino)-5-methoxy-3,4-dihydro-2H-chromen-8-yl]-2-methylbenzenesulfonamide,
N-[(3R)-3-(dimethylamino)-5-methoxy-3,4-dihydro-2H-chromen-8-yl]-2,3,5,6-tetramethylbenzenesulfonamide,
N-[(3R)-3-(dimethylamino)-5-methoxy-3,4-dihydro-2H-chromen-8-yl]-2-fluorobenzenesulfonamide,
N-[(3R)-3-(dimethylamino)-5-methoxy-3,4-dihydro-2H-chromen-8-yl]-4-fluorobenzenesulfonamide,
N-[(3R)-3-(dimethylamino)-5-methoxy-3,4-dihydro-2H-chromen-8-yl]-2-(trifluoromethyl)benzenesulfonamide,
2,5-dichloro-N-[(3R)-3-(dimethylamino)-5-methoxy-3,4-dihydro-2H-chromen-8-yl]thiophene-3-sulfonamide,
N-[(3R)-3-(dimethylamino)-5-methoxy-3,4-dihydro-2H-chromen-8-yl]-3,4-dimethoxybenzenesulfonamide,
N-[(3R)-3-(dimethylamino)-5-methoxy-3,4-dihydro-2H-chromen-8-yl]-2,5-dimethylbenzenesulfonamide,
N-[(3R)-3-(dimethylamino)-5-methoxy-3,4-dihydro-2H-chromen-8-yl]-3-methoxybenzenesulfonamide,
N-[(3R)-3-(dimethylamino)-5-methoxy-3,4-dihydro-2H-chromen-8-yl]-2,5-difluorobenzenesulfonamide,
N-[(3R)-3-(dimethylamino)-5-methoxy-3,4-dihydro-2H-chromen-8-yl]-4-[2-(phenylsulfonyl)ethyl]benzenesulfonamide,
8-chloro-N-[(3R)-3-(dimethylamino)-5-methoxy-3,4-dihydro-2H-chromen-8-yl]naphthalene-2-sulfonamide,
N-[4-({[(3R)-3-(dimethylamino)-5-methoxy-3,4-dihydro-2H-chromen-8-yl]amino}sulfonyl)phenyl]-2,2,2-trifluoroacetamide,
N-[(3R)-3-(dimethylamino)-5-methoxy-3,4-dihydro-2H-chromen-8-yl]-2-(phenylsulfonyl)benzenesulfonamide,
7-bromo-N-[(3R)-3-(dimethylamino)-5-methoxy-3,4-dihydro-2H-chromen-8-yl]naphthalene-1-sulfonamide,
4-(1,3-benzoxazol-2-yl)-N-[(3R)-3-(dimethylamino)-5-methoxy-3,4-dihydro-2H-chromen-8-yl]benzenesulfonamide,
N-[(3R)-3-(dimethylamino)-5-methoxy-3,4-dihydro-2H-chromen-8-yl]-4-methylnaphthalene-1-sulfonamide,
5-chloro-N-[(3R)-3-(dimethylamino)-5-methoxy-3,4-dihydro-2H-chromen-8-yl]naphthalene-2-sulfonamide,
N-[(3R)-3-(dimethylamino)-5-methoxy-3,4-dihydro-2H-chromen-8-yl]-1,2-dimethyl-1H-imidazole-4-sulfonamide,
N-[(3R)-3-(dimethylamino)-5-methoxy-3,4-dihydro-2H-chromen-8-yl]thiophene-3-sulfonamide,
2-chloro-N-[(3R)-3-(dimethylamino)-5-methoxy-3,4-dihydro-2H-chromen-8-yl]-4,5-difluorobenzenesulfonamide,
N-[(3R)-3-(dimethylamino)-5-methoxy-3,4-dihydro-2H-chromen-8-yl]-4-(methylsulfonyl)benzenesulfonamide,
4-chloro-N-[(3R)-3-(dimethylamino)-5-methoxy-3,4-dihydro-2H-chromen-8-yl]-2,5-difluorobenzenesulfonamide,
N-[(3R)-3-(dimethylamino)-5-methoxy-3,4-dihydro-2H-chromen-8-yl]-1,1′-biphenyl-4-sulfonamide,
2-chloro-4-cyano-N-[(3R)-3-(dimethylamino)-5-methoxy-3,4-dihydro-2H-chromen-8-yl]benzenesulfonamide,
3-chloro-N-[(3R)-3-(dimethylamino)-5-methoxy-3,4-dihydro-2H-chromen-8-yl]-4-methylbenzenesulfonamide,
4-bromo-N-[(3R)-3-(dimethylamino)-5-methoxy-3,4-dihydro-2H-chromen-8-yl]-2-methylbenzenesulfonamide,
4-chloro-N-[(3R)-3-(dimethylamino)-5-methoxy-3,4-dihydro-2H-chromen-8-yl]-2,5-dimethylbenzenesulfonamide,
N-[(3R)-3-(dimethylamino)-5-methoxy-3,4-dihydro-2H-chromen-8-yl]-2,3,4-trifluorobenzenesulfonamide,
4-butyl-N-[(3R)-3-(dimethylamino)-5-methoxy-3,4-dihydro-2H-chromen-8-yl]benzenesulfonamide,
1-(3-chlorophenyl)-N-[(3R)-3-(dimethylamino)-5-methoxy-3,4-dihydro-2H-chromen-8-yl]methanesulfonamide,
N-[(3R)-3-(dimethylamino)-5-methoxy-3,4-dihydro-2H-chromen-8-yl]-2,4,5-trifluorobenzenesulfonamide,
methyl 4-({[(3R)-3-(dimethylamino)-5-methoxy-3,4-dihydro-2H-chromen-8-yl]amino}sulfonyl)-2,5-dimethyl-3-furoate,
5-bromo-6-chloro-N-[(3R)-3-(dimethylamino)-5-methoxy-3,4-dihydro-2H-chromen-8-yl]pyridine-3-sulfonamide,
3-chloro-N-[(3R)-3-(dimethylamino)-5-methoxy-3,4-dihydro-2H-chromen-8-yl]-5-fluoro-2-methylbenzenesulfonamide,
4-bromo-N-[(3R)-3-(dimethylamino)-5-methoxy-3,4-dihydro-2H-chromen-8-yl]-2-ethylbenzenesulfonamide,
N-[(3R)-3-(dimethylamino)-5-methoxy-3,4-dihydro-2H-chromen-8-yl]-6-phenoxypyridine-3-sulfonamide,
2,3,4-trichloro-N-[(3R)-3-(dimethylamino)-5-methoxy-3,4-dihydro-2H-chromen-8-yl]benzenesulfonamide,
4-bromo-N-[(3R)-3-(dimethylamino)-5-methoxy-3,4-dihydro-2H-chromen-8-yl]-2,5-difluorobenzenesulfonamide,
N-[(3R)-3-(dimethylamino)-5-methoxy-3,4-dihydro-2H-chromen-8-yl]-1,1′-biphenyl-3-sulfonamide,
N-[(3R)-3-(dimethylamino)-5-methoxy-3,4-dihydro-2H-chromen-8-yl]-1,1′-biphenyl-2-sulfonamide,
N-[(3R)-3-(dimethylamino)-5-methoxy-3,4-dihydro-2H-chromen-8-yl]-1-pyridin-3-ylmethanesulfonamide,
N-[(3R)-3-(dimethylamino)-5-methoxy-3,4-dihydro-2H-chromen-8-yl]-2,2-diphenylethanesulfonamide,
N-[(3R)-3-(dimethylamino)-5-methoxy-3,4-dihydro-2H-chromen-8-yl]-1-benzofuran-2-sulfonamide,
4-chloro-N′-[(3R)-3-(dimethylamino)-5-methoxy-3,4-dihydro-2H-chromen-8-yl]benzene-1,3-disulfonamide,
N-[(3R)-3-(dimethylamino)-5-methoxy-3,4-dihydro-2H-chromen-8-yl]-4-pentylbenzenesulfonamide,
N-[(3R)-3-(dimethylamino)-5-methoxy-3,4-dihydro-2H-chromen-8-yl]-3-(2-methoxyphenoxy)benzenesulfonamide,
N-[(3R)-3-(dimethylamino)-5-methoxy-3,4-dihydro-2H-chromen-8-yl]-4′-methoxy-1,1′-biphenyl-3-sulfonamide,
N-[(3R)-3-(dimethylamino)-5-methoxy-3,4-dihydro-2H-chromen-8-yl]cyclopropanesulfonamide,
1-[3,5-bis(trifluoromethyl)phenyl]-N-[(3R)-3-(dimethylamino)-5-methoxy-3,4-dihydro-2H-chromen-8-yl]methanesulfonamide,
N-[(3R)-3-(dimethylamino)-5-methoxy-3,4-dihydro-2H-chromen-8-yl]-4-fluoronaphthalene-1-sulfonamide,
N-[(3R)-3-(dimethylamino)-5-methoxy-3,4-dihydro-2H-chromen-8-yl]-3,5-difluorobenzenesulfonamide,
N-[(3R)-3-(dimethylamino)-5-methoxy-3,4-dihydro-2H-chromen-8-yl]-3-fluoro-4-methoxybenzenesulfonamide,
N-[(3R)-3-(dimethylamino)-5-methoxy-3,4-dihydro-2H-chromen-8-yl]-5-[2-(methylthio)pyrimidin-4-yl]thiophene-2-sulfonamide,
1-[3-chloro-5-(trifluoromethyl)pyridin-2-yl]-N-[(3R)-3-(dimethylamino)-5-methoxy-3,4-dihydro-2H-chromen-8-yl]-1H-pyrrole-2-sulfonamide,
2,6-dichloro-N-[(3R)-3-(dimethylamino)-5-methoxy-3,4-dihydro-2H-chromen-8-yl]-4-(trifluoromethyl)benzenesulfonamide,
N-[(3R)-3-(dimethylamino)-5-methoxy-3,4-dihydro-2H-chromen-8-yl]-5-[1-methyl-3-(trifluoromethyl)-1H-pyrazol-5-yl]thiophene-2-sulfonamide,
N-[(3R)-3-(dimethylamino)-5-methoxy-3,4-dihydro-2H-chromen-8-yl]-5-[5-(trifluoromethyl)isoxazol-3-yl]thiophene-2-sulfonamide,
N-[(3R)-3-(dimethylamino)-5-methoxy-3,4-dihydro-2H-chromen-8-yl]-4-fluoro-2-(trifluoromethyl)benzenesulfonamide,
N-[(3R)-3-(dimethylamino)-5-methoxy-3,4-dihydro-2H-chromen-8-yl]-4-fluoro-3-(trifluoromethyl)benzenesulfonamide,
N-[(3R)-3-(dimethylamino)-5-methoxy-3,4-dihydro-2H-chromen-8-yl]-2,4,6-trifluorobenzenesulfonamide,
N-[(3R)-3-(dimethylamino)-5-methoxy-3,4-dihydro-2H-chromen-8-yl]-5-isoxazol-5-ylthiophene-2-sulfonamide,
N-[(3R)-3-(dimethylamino)-5-methoxy-3,4-dihydro-2H-chromen-8-yl]-1-(3-nitrophenyl)methanesulfonamide,
N-[(3R)-3-(dimethylamino)-5-methoxy-3,4-dihydro-2H-chromen-8-yl]-2-fluoro-5-(trifluoromethyl)benzenesulfonamide,
N-[(3R)-3-(dimethylamino)-5-methoxy-3,4-dihydro-2H-chromen-8-yl]-5-methyl-2,1,3-benzothiadiazole-4-sulfonamide,
N-[(3R)-3-(dimethylamino)-5-methoxy-3,4-dihydro-2H-chromen-8-yl]-5-fluoro-3-methyl-1-benzothiophene-2-sulfonamide,
2,3-dichloro-N-[(3R)-3-(dimethylamino)-5-methoxy-3,4-dihydro-2H-chromen-8-yl]-4-methoxybenzenesulfonamide,
1-(4-chlorophenyl)-N-[(3R)-3-(dimethylamino)-5-methoxy-3,4-dihydro-2H-chromen-8-yl]methanesulfonamide,
2,3-dichloro-N-[(3R)-3-(dimethylamino)-5-methoxy-3,4-dihydro-2H-chromen-8-yl]benzenesulfonamide,
5-chloro-N-[(3R)-3-(dimethylamino)-5-methoxy-3,4-dihydro-2H-chromen-8-yl]thiophene-2-sulfonamide,
2-chloro-N-[(3R)-3-(dimethylamino)-5-methoxy-3,4-dihydro-2H-chromen-8-yl]-6-methylbenzenesulfonamide,
3,4-dichloro-N-[(3R)-3-(dimethylamino)-5-methoxy-3,4-dihydro-2H-chromen-8-yl]benzenesulfonamide,
3,5-dichloro-N-[(3R)-3-(dimethylamino)-5-methoxy-3,4-dihydro-2H-chromen-8-yl]benzenesulfonamide,
4-(3-chloro-2-cyanophenoxy)-N-[(3R)-3-(dimethylamino)-5-methoxy-3,4-dihydro-2H-chromen-8-yl]benzenesulfonamide,
5-bromo-N-[(3R)-3-(dimethylamino)-5-methoxy-3,4-dihydro-2H-chromen-8-yl]thiophene-2-sulfonamide,
N-[(3R)-3-(dimethylamino)-5-methoxy-3,4-dihydro-2H-chromen-8-yl]-4-isopropylbenzenesulfonamide,
4-bromo-5-chloro-N-[(3R)-3-(dimethylamino)-5-methoxy-3,4-dihydro-2H-chromen-8-yl]thiophene-2-sulfonamide,
5-chloro-N-[(3R)-3-(dimethylamino)-5-methoxy-3,4-dihydro-2H-chromen-8-yl]-2-methoxybenzenesulfonamide,
N-[(3R)-3-(dimethylamino)-5-methoxy-3,4-dihydro-2H-chromen-8-yl]-3-fluorobenzenesulfonamide,
N-[2-chloro-4-({[(3R)-3-(dimethylamino)-5-methoxy-3,4-dihydro-2H-chromen-8-yl]amino}sulfonyl)phenyl]acetamide,
2,4-dichloro-N-[(3R)-3-(dimethylamino)-5-methoxy-3,4-dihydro-2H-chromen-8-yl]-5-methylbenzenesulfonamide,
N-[(3R)-3-(dimethylamino)-5-methoxy-3,4-dihydro-2H-chromen-8-yl]-2-oxo-1,2,3,4-tetrahydroquinoline-6-sulfonamide,
2,4-dichloro-N-[(3R)-3-(dimethylamino)-5-methoxy-3,4-dihydro-2H-chromen-8-yl]-6-methylbenzenesulfonamide,
N-[(3R)-3-(dimethylamino)-5-methoxy-3,4-dihydro-2H-chromen-8-yl]-3,4-difluorobenzenesulfonamide,
N-[(3R)-3-(dimethylamino)-5-methoxy-3,4-dihydro-2H-chromen-8-yl]-2-methylbenzenesulfonamide,
N-[(3R)-3-(dimethylamino)-5-methoxy-3,4-dihydro-2H-chromen-8-yl]-4-iodobenzenesulfonamide,
3-Chloro-N-[(3R)-5-methoxy-3-pyrrolidin-1-yl-3,4-dihydro-2H-chromen-8-yl]-4-methylbenzenesulfonamide, and
5-Chloro-N-[(3R)-5-methoxy-3-pyrrolidin-1-yl-3,4-dihydro-2H-chromen-8-yl]naphthalene-2-sulfonamide, or salts, solvates or solvated salts thereof.

A further embodiment of the invention relates to compounds selected from the group consisting of
(2S)-5-{[(3-Bromophenyl)sulfonyl]amino}-N,N-dimethyl-1,2,3,4-tetrahydronaphthalen-2-ammonium acetate,
N-[(6S)-4-Bromo-6-(dimethylamino)-5,6,7,8-tetrahydronaphthalen-1-yl]benzenesulfonamide,
N-[(6S)-4-Bromo-6-(dimethylamino)-5,6,7,8-tetrahydronaphthalen-1-yl]-3-chloro-4-fluorobenzenesulfonamide,
4-bromo-N-[(6S)-6-(dimethylamino)-4-methoxy-5,6,7,8-tetrahydronaphthalen-1-yl]-2-ethylbenzenesulfonamide,
5-bromo-6-chloro-N-[(6S)-6-(dimethylamino)-4-methoxy-5,6,7,8-tetrahydronaphthalen-1-yl]pyridine-3-sulfonamide,
N-[(6S)-6-(dimethylamino)-4-methoxy-5,6,7,8-tetrahydronaphthalen-1-yl]-2,3-dihydro-1,4-benzodioxine-6-sulfonamide,
N-[(6S)-6-(dimethylamino)-4-methoxy-5,6,7,8-tetrahydronaphthalen-1-yl]-1,1′-biphenyl-2-sulfonamide,
N-[(6S)-6-(dimethylamino)-4-methoxy-5,6,7,8-tetrahydronaphthalen-1-yl]-1-pyridin-3-ylmethanesulfonamide,
4-chloro-N′-[(6S)-6-(dimethylamino)-4-methoxy-5,6,7,8-tetrahydronaphthalen-1-yl]benzene-1,3-disulfonamide,
5-chloro-N-[(6S)-6-(dimethylamino)-4-methoxy-5,6,7,8-tetrahydronaphthalen-1-yl]naphthalene-1-sulfonamide,
N-[(6S)-6-(dimethylamino)-4-methoxy-5,6,7,8-tetrahydronaphthalen-1-yl]-4-fluoro-3-(trifluoromethyl)benzenesulfonamide,
N-[(6S)-6-(dimethylamino)-4-methoxy-5,6,7,8-tetrahydronaphthalen-1-yl]-5-fluoro-3-methyl-1-benzothiophene-2-sulfonamide,
1-(4-chlorophenyl)-N-[(6S)-6-(dimethylamino)-4-methoxy-5,6,7,8-tetrahydronaphthalen-1-yl]methanesulfonamide,
2-chloro-4-cyano-N-[(6S)-6-(dimethylamino)-4-methoxy-5,6,7,8-tetrahydronaphthalen-1-yl]benzenesulfonamide,
6-chloro-N-[(6S)-6-(dimethylamino)-4-methoxy-5,6,7,8-tetrahydronaphthalen-1-yl]imidazo[2,1-b][1,3]thiazole-5-sulfonamide,
N-[(6S)-6-(dimethylamino)-4-methoxy-5,6,7,8-tetrahydronaphthalen-1-yl]-2-(methylsulfonyl)benzenesulfonamide,
7-chloro-N-[(6S)-6-(dimethylamino)-4-methoxy-5,6,7,8-tetrahydronaphthalen-1-yl]-2,1,3-benzoxadiazole-4-sulfonamide,
4,5-dibromo-N-[(6S)-6-(dimethylamino)-4-methoxy-5,6,7,8-tetrahydronaphthalen-1-yl]thiophene-2-sulfonamide,
5-bromo-N-[(6S)-6-(dimethylamino)-4-methoxy-5,6,7,8-tetrahydronaphthalen-1-yl]-2-methoxybenzenesulfonamide,
N-[(6S)-6-(dimethylamino)-4-methoxy-5,6,7,8-tetrahydronaphthalen-1-yl]-4-phenoxybenzenesulfonamide,
1-acetyl-5-bromo-N-[(6S)-6-(dimethylamino)-4-methoxy-5,6,7,8-tetrahydronaphthalen-1-yl]indoline-6-sulfonamide,
N-[(6S)-6-(dimethylamino)-4-methoxy-5,6,7,8-tetrahydronaphthalen-1-yl]-4-propylbenzenesulfonamide,
4-cyano-N-[(6S)-6-(dimethylamino)-4-methoxy-5,6,7,8-tetrahydronaphthalen-1-yl]benzenesulfonamide,
5-chloro-N-[(6S)-6-(dimethylamino)-4-methoxy-5,6,7,8-tetrahydronaphthalen-1-yl]thiophene-2-sulfonamide,
N-[(6S)-6-(dimethylamino)-4-methoxy-5,6,7,8-tetrahydronaphthalen-1-yl]naphthalene-2-sulfonamide,
3-chloro-N-[(6S)-6-(dimethylamino)-4-methoxy-5,6,7,8-tetrahydronaphthalen-1-yl]-4-methylbenzenesulfonamide,
4-bromo-N-[(6S)-6-(dimethylamino)-4-methoxy-5,6,7,8-tetrahydronaphthalen-1-yl]-2-methylbenzenesulfonamide,
4-chloro-N-[(6S)-6-(dimethylamino)-4-methoxy-5,6,7,8-tetrahydronaphthalen-1-yl]-2,5-dimethylbenzenesulfonamide,
N-[(6S)-6-(dimethylamino)-4-methoxy-5,6,7,8-tetrahydronaphthalen-1-yl]-2,3,4-trifluorobenzenesulfonamide,
1-(3-chlorophenyl)-N-[(6S)-6-(dimethylamino)-4-methoxy-5,6,7,8-tetrahydronaphthalen-1-yl]methanesulfonamide,
N-[(6S)-6-(dimethylamino)-4-methoxy-5,6,7,8-tetrahydronaphthalen-1-yl]-2,4,5-trifluorobenzenesulfonamide,
3-chloro-N-[(6S)-6-(dimethylamino)-4-methoxy-5,6,7,8-tetrahydronaphthalen-1-yl]-5-fluoro-2-methylbenzenesulfonamide,
N-[(6S)-6-(dimethylamino)-4-methoxy-5,6,7,8-tetrahydronaphthalen-1-yl]-6-phenoxypyridine-3-sulfonamide,
4-bromo-N-[(6S)-6-(dimethylamino)-4-methoxy-5,6,7,8-tetrahydronaphthalen-1-yl]-2,5-difluorobenzenesulfonamide,
N-[(6S)-6-(dimethylamino)-4-methoxy-5,6,7,8-tetrahydronaphthalen-1-yl]-1-pyridin-2-ylmethanesulfonamide,
N-[(6S)-6-(dimethylamino)-4-methoxy-5,6,7,8-tetrahydronaphthalen-1-yl]-1,1′-biphenyl-3-sulfonamide,
N-[(6S)-6-(dimethylamino)-4-methoxy-5,6,7,8-tetrahydronaphthalen-1-yl]-1-benzofuran-2-sulfonamide,
4-chloro-N′-[(6S)-6-(dimethylamino)-4-methoxy-5,6,7,8-tetrahydronaphthalen-1-yl]benzene-1,3-disulfonamide,
N-[(6S)-6-(dimethylamino)-4-methoxy-5,6,7,8-tetrahydronaphthalen-1-yl]-3-(2-methoxyphenoxy)benzenesulfonamide,
N-[(6S)-6-(dimethylamino)-4-methoxy-5,6,7,8-tetrahydronaphthalen-1-yl]-4′-methoxy-1,1′-biphenyl-3-sulfonamide,
N-[(6S)-6-(dimethylamino)-4-methoxy-5,6,7,8-tetrahydronaphthalen-1-yl]cyclopropanesulfonamide,
N-[(6S)-6-(dimethylamino)-4-methoxy-5,6,7,8-tetrahydronaphthalen-1-yl]-4-fluoronaphthalene-1-sulfonamide,
N-[(6S)-6-(dimethylamino)-4-methoxy-5,6,7,8-tetrahydronaphthalen-1-yl]-3,5-difluorobenzenesulfonamide,
N-[(6S)-6-(dimethylamino)-4-methoxy-5,6,7,8-tetrahydronaphthalen-1-yl]-3-fluoro-4-methoxybenzenesulfonamide,
1-[3-chloro-5-(trifluoromethyl)pyridin-2-yl]-N-[(6S)-6-(dimethylamino)-4-methoxy-5,6,7,8-tetrahydronaphthalen-1-yl]-1H-pyrrole-2-sulfonamide,
N-[(6S)-6-(dimethylamino)-4-methoxy-5,6,7,8-tetrahydronaphthalen-1-yl]-5-[5-(trifluoromethyl)isoxazol-3-yl]thiophene-2-sulfonamide,
N-[(6S)-6-(dimethylamino)-4-methoxy-5,6,7,8-tetrahydronaphthalen-1-yl]-2,4,6-trifluorobenzenesulfonamide,
N-[(6S)-6-(dimethylamino)-4-methoxy-5,6,7,8-tetrahydronaphthalen-1-yl]-5-isoxazol-5-ylthiophene-2-sulfonamide,
N-[(6S)-6-(dimethylamino)-4-methoxy-5,6,7,8-tetrahydronaphthalen-1-yl]-1-(3-nitrophenyl)methanesulfonamide,
N-[(6S)-6-(dimethylamino)-4-methoxy-5,6,7,8-tetrahydronaphthalen-1-yl]-2-fluoro-5-(trifluoromethyl)benzenesulfonamide,
2,3-dichloro-N-[(6S)-6-(dimethylamino)-4-methoxy-5,6,7,8-tetrahydronaphthalen-1-yl]-4-methoxybenzenesulfonamide,
N-[(6S)-6-(dimethylamino)-4-methoxy-5,6,7,8-tetrahydronaphthalen-1-yl]-4-methylbenzenesulfonamide,
5-(dimethylamino)-N-[(6S)-6-(dimethylamino)-4-methoxy-5,6,7,8-tetrahydronaphthalen-1-yl]naphthalene-1-sulfonamide,
N-[(6S)-6-(dimethylamino)-4-methoxy-5,6,7,8-tetrahydronaphthalen-1-yl]-2-nitrobenzenesulfonamide,
N-[(6S)-6-(dimethylamino)-4-methoxy-5,6,7,8-tetrahydronaphthalen-1-yl]-4-nitrobenzenesulfonamide,
N-[(6S)-6-(dimethylamino)-4-methoxy-5,6,7,8-tetrahydronaphthalen-1-yl]benzenesulfonamide,
N-[(6S)-6-(dimethylamino)-4-methoxy-5,6,7,8-tetrahydronaphthalen-1-yl]-3,4,5-trimethoxybenzenesulfonamide,
N-[(6S)-6-(dimethylamino)-4-methoxy-5,6,7,8-tetrahydronaphthalen-1-yl]-1-phenylmethanesulfonamide,
N-[(6S)-6-(dimethylamino)-4-methoxy-5,6,7,8-tetrahydronaphthalen-1-yl]-4-fluorobenzenesulfonamide,
N-[(6S)-6-(dimethylamino)-4-methoxy-5,6,7,8-tetrahydronaphthalen-1-yl]-4-isopropylbenzenesulfonamide,
N-[(6S)-6-(dimethylamino)-4-methoxy-5,6,7,8-tetrahydronaphthalen-1-yl]-4-iodobenzenesulfonamide,
3-bromo-5-chloro-N-[(6S)-6-(dimethylamino)-4-methoxy-5,6,7,8-tetrahydronaphthalen-1-yl]thiophene-2-sulfonamide,
4-tert-butyl-N-[(6S)-6-(dimethylamino)-4-methoxy-5,6,7,8-tetrahydronaphthalen-1-yl]benzenesulfonamide,
N-[(6S)-6-(dimethylamino)-4-methoxy-5,6,7,8-tetrahydronaphthalen-1-yl]-4-methoxybenzenesulfonamide,
2-chloro-N-[(6S)-6-(dimethylamino)-4-methoxy-5,6,7,8-tetrahydronaphthalen-1-yl]benzenesulfonamide,
N-[4-({[(6S)-6-(dimethylamino)-4-methoxy-5,6,7,8-tetrahydronaphthalen-1-yl]amino}sulfonyl)phenyl]acetamide,
2-bromo-N-[(6S)-6-(dimethylamino)-4-methoxy-5,6,7,8-tetrahydronaphthalen-1-yl]benzenesulfonamide,
N-{[5-({[(6S)-6-(dimethylamino)-4-methoxy-5,6,7,8-tetrahydronaphthalen-1-yl]amino}sulfonyl)thien-2-yl]methyl}benzamide,
N-[(6S)-6-(dimethylamino)-4-methoxy-5,6,7,8-tetrahydronaphthalen-1-yl]-4-(trifluoromethyl)benzenesulfonamide,
N-[(6S)-6-(dimethylamino)-4-methoxy-5,6,7,8-tetrahydronaphthalen-1-yl]-4-ethylbenzenesulfonamide,
2-chloro-N-[(6S)-6-(dimethylamino)-4-methoxy-5,6,7,8-tetrahydronaphthalen-1-yl]-5-(trifluoromethyl)benzenesulfonamide,
N-[(6S)-6-(dimethylamino)-4-methoxy-5,6,7,8-tetrahydronaphthalen-1-yl]-4-methyl-3-nitrobenzenesulfonamide,
N-[(6S)-6-(dimethylamino)-4-methoxy-5,6,7,8-tetrahydronaphthalen-1-yl]naphthalene-1-sulfonamide,
4-chloro-N-[(6S)-6-(dimethylamino)-4-methoxy-5,6,7,8-tetrahydronaphthalen-1-yl]-3-nitrobenzenesulfonamide,
N-[(6S)-6-(dimethylamino)-4-methoxy-5,6,7,8-tetrahydronaphthalen-1-yl]-3-(trifluoromethyl)benzenesulfonamide,
4-chloro-N-[(6S)-6-(dimethylamino)-4-methoxy-5,6,7,8-tetrahydronaphthalen-1-yl]benzenesulfonamide,
2,4-dichloro-N-[(6S)-6-(dimethylamino)-4-methoxy-5,6,7,8-tetrahydronaphthalen-1-yl]benzenesulfonamide,
N-[5-({[(6S)-6-(dimethylamino)-4-methoxy-5,6,7,8-tetrahydronaphthalen-1-yl]amino}sulfonyl)-4-methyl-1,3-thiazol-2-yl]acetamide,
N-[(6S)-6-(dimethylamino)-4-methoxy-5,6,7,8-tetrahydronaphthalen-1-yl]thiophene-2-sulfonamide,
3,5-dichloro-N-[(6S)-6-(dimethylamino)-4-methoxy-5,6,7,8-tetrahydronaphthalen-1-yl]-2-hydroxybenzenesulfonamide,
N-[(6S)-6-(dimethylamino)-4-methoxy-5,6,7,8-tetrahydronaphthalen-1-yl]-3-nitrobenzenesulfonamide,
N-[(6S)-6-(dimethylamino)-4-methoxy-5,6,7,8-tetrahydronaphthalen-1-yl]-2,5-dimethoxybenzenesulfonamide,
N-[(6S)-6-(dimethylamino)-4-methoxy-5,6,7,8-tetrahydronaphthalen-1-yl]-5-(phenylsulfonyl)thiophene-2-sulfonamide,
N-[(6S)-6-(dimethylamino)-4-methoxy-5,6,7,8-tetrahydronaphthalen-1-yl]-5-pyridin-2-ylthiophene-2-sulfonamide,
2-cyano-N-[(6S)-6-(dimethylamino)-4-methoxy-5,6,7,8-tetrahydronaphthalen-1-yl]benzenesulfonamide,
5-chloro-N-[(6S)-6-(dimethylamino)-4-methoxy-5,6,7,8-tetrahydronaphthalen-1-yl]-1,3-dimethyl-1H-pyrazole-4-sulfonamide,
N-[(6S)-6-(dimethylamino)-4-methoxy-5,6,7,8-tetrahydronaphthalen-1-yl]-3,5-dimethylisoxazole-4-sulfonamide,
N-[(6S)-6-(dimethylamino)-4-methoxy-5,6,7,8-tetrahydronaphthalen-1-yl]-1-methyl-1H-imidazole-4-sulfonamide,
N-[(6S)-6-(dimethylamino)-4-methoxy-5,6,7,8-tetrahydronaphthalen-1-yl]-5-isoxazol-3-ylthiophene-2-sulfonamide,
methyl 3-({[(6S)-6-(dimethylamino)-4-methoxy-5,6,7,8-tetrahydronaphthalen-1-yl]amino}sulfonyl)thiophene-2-carboxylate,
2,6-dichloro-N-[(6S)-6-(dimethylamino)-4-methoxy-5,6,7,8-tetrahydronaphthalen-1-yl]benzenesulfonamide,
N-[(6S)-6-(dimethylamino)-4-methoxy-5,6,7,8-tetrahydronaphthalen-1-yl]-2,6-difluorobenzenesulfonamide,
N-[(6S)-6-(dimethylamino)-4-methoxy-5,6,7,8-tetrahydronaphthalen-1-yl]-2-methyl-5-nitrobenzenesulfonamide,
N-[(6S)-6-(dimethylamino)-4-methoxy-5,6,7,8-tetrahydronaphthalen-1-yl]-3-methylbenzenesulfonamide,
4-bromo-N-[(6S)-6-(dimethylamino)-4-methoxy-5,6,7,8-tetrahydronaphthalen-1-yl]-2-fluorobenzenesulfonamide,
N-[3-({[(6S)-6-(dimethylamino)-4-methoxy-5,6,7,8-tetrahydronaphthalen-1-yl]amino}sulfonyl)phenyl]acetamide,
N-[(6S)-6-(dimethylamino)-4-methoxy-5,6,7,8-tetrahydronaphthalen-1-yl]-2-methyl-4-nitrobenzenesulfonamide,
3-chloro-N-[(6S)-6-(dimethylamino)-4-methoxy-5,6,7,8-tetrahydronaphthalen-1-yl]benzenesulfonamide,
2-chloro-N-[(6S)-6-(dimethylamino)-4-methoxy-5,6,7,8-tetrahydronaphthalen-1-yl]-4-fluorobenzenesulfonamide,
3-chloro-N-[(6S)-6-(dimethylamino)-4-methoxy-5,6,7,8-tetrahydronaphthalen-1-yl]-4-fluorobenzenesulfonamide,
2-chloro-N-[(6S)-6-(dimethylamino)-4-methoxy-5,6,7,8-tetrahydronaphthalen-1-yl]-4-(trifluoromethyl)benzenesulfonamide,
N-[(6S)-6-(dimethylamino)-4-methoxy-5,6,7,8-tetrahydronaphthalen-1-yl]-2,4-difluorobenzenesulfonamide,
N-[(6S)-6-(dimethylamino)-4-methoxy-5,6,7,8-tetrahydronaphthalen-1-yl]-5-fluoro-2-methylbenzenesulfonamide,
2,5-dichloro-N-[(6S)-6-(dimethylamino)-4-methoxy-5,6,7,8-tetrahydronaphthalen-1-yl]benzenesulfonamide,
3-bromo-N-[(6S)-6-(dimethylamino)-4-methoxy-5,6,7,8-tetrahydronaphthalen-1-yl]benzenesulfonamide,
3-chloro-N-[(6S)-6-(dimethylamino)-4-methoxy-5,6,7,8-tetrahydronaphthalen-1-yl]-2-methylbenzenesulfonamide,
N-[(6S)-6-(dimethylamino)-4-methoxy-5,6,7,8-tetrahydronaphthalen-1-yl]-2-fluorobenzenesulfonamide,
N-[(6S)-6-(dimethylamino)-4-methoxy-5,6,7,8-tetrahydronaphthalen-1-yl]-2-(trifluoromethyl)benzenesulfonamide,
2,5-dichloro-N-[(6S)-6-(dimethylamino)-4-methoxy-5,6,7,8-tetrahydronaphthalen-1-yl]thiophene-3-sulfonamide,
N-[(6S)-6-(dimethylamino)-4-methoxy-5,6,7,8-tetrahydronaphthalen-1-yl]-3,4-dimethoxybenzenesulfonamide,
2,3-dichloro-N-[(6S)-6-(dimethylamino)-4-methoxy-5,6,7,8-tetrahydronaphthalen-1-yl]benzenesulfonamide,
2-chloro-N-[(6S)-6-(dimethylamino)-4-methoxy-5,6,7,8-tetrahydronaphthalen-1-yl]-6-methylbenzenesulfonamide,
3,4-dichloro-N-[(6S)-6-(dimethylamino)-4-methoxy-5,6,7,8-tetrahydronaphthalen-1-yl]benzenesulfonamide,
3,5-dichloro-N-[(6S)-6-(dimethylamino)-4-methoxy-5,6,7,8-tetrahydronaphthalen-1-yl]benzenesulfonamide,
5-bromo-N-[(6S)-6-(dimethylamino)-4-methoxy-5,6,7,8-tetrahydronaphthalen-1-yl]thiophene-2-sulfonamide,
4-bromo-5-chloro-N-[(6S)-6-(dimethylamino)-4-methoxy-5,6,7,8-tetrahydronaphthalen-1-yl]thiophene-2-sulfonamide,
5-chloro-N-[(6S)-6-(dimethylamino)-4-methoxy-5,6,7,8-tetrahydronaphthalen-1-yl]-2-methoxybenzenesulfonamide,
N-[(6S)-6-(dimethylamino)-4-methoxy-5,6,7,8-tetrahydronaphthalen-1-yl]-3-fluorobenzenesulfonamide,
N-[2-chloro-4-({[(6S)-6-(dimethylamino)-4-methoxy-5,6,7,8-tetrahydronaphthalen-1-yl]amino}sulfonyl)phenyl]acetamide,
N-[(6S)-6-(dimethylamino)-4-methoxy-5,6,7,8-tetrahydronaphthalen-1-yl]-2-oxo-1,2,3,4-tetrahydroquinoline-6-sulfonamide,
N-[(6S)-6-(dimethylamino)-4-methoxy-5,6,7,8-tetrahydronaphthalen-1-yl]-3,4-difluorobenzenesulfonamide,
N-[(6S)-6-(dimethylamino)-4-methoxy-5,6,7,8-tetrahydronaphthalen-1-yl]-2-methylbenzenesulfonamide,
N-[(6S)-6-(dimethylamino)-4-methoxy-5,6,7,8-tetrahydronaphthalen-1-yl]-2,5-dimethylbenzenesulfonamide,
N-[(6S)-6-(dimethylamino)-4-methoxy-5,6,7,8-tetrahydronaphthalen-1-yl]-3-methoxybenzenesulfonamide,
N-[(6S)-6-(dimethylamino)-4-methoxy-5,6,7,8-tetrahydronaphthalen-1-yl]-2,5-difluorobenzenesulfonamide,
4-chloro-N-[(6S)-6-(dimethylamino)-4-methoxy-5,6,7,8-tetrahydronaphthalen-1-yl]naphthalene-1-sulfonamide,
N-[(6S)-6-(dimethylamino)-4-methoxy-5,6,7,8-tetrahydronaphthalen-1-yl]-4-[2-(phenylsulfonyl)ethyl]benzenesulfonamide,
8-chloro-N-[(6S)-6-(dimethylamino)-4-methoxy-5,6,7,8-tetrahydronaphthalen-1-yl]naphthalene-2-sulfonamide,
N-[4-({[(6S)-6-(dimethylamino)-4-methoxy-5,6,7,8-tetrahydronaphthalen-1-yl]amino}sulfonyl)phenyl]-2,2,2-trifluoroacetamide,
N-[(6S)-6-(dimethylamino)-4-methoxy-5,6,7,8-tetrahydronaphthalen-1-yl]-2-(phenylsulfonyl)benzenesulfonamide,
7-bromo-N-[(6S)-6-(dimethylamino)-4-methoxy-5,6,7,8-tetrahydronaphthalen-1-yl]naphthalene-1-sulfonamide,
4-(1,3-benzoxazol-2-yl)-N-[(6S)-6-(dimethylamino)-4-methoxy-5,6,7,8-tetrahydronaphthalen-1-yl]benzenesulfonamide,
N-[(6S)-6-(dimethylamino)-4-methoxy-5,6,7,8-tetrahydronaphthalen-1-yl]-4-methylnaphthalene-1-sulfonamide,
5-chloro-N-[(6S)-6-(dimethylamino)-4-methoxy-5,6,7,8-tetrahydronaphthalen-1-yl]naphthalene-2-sulfonamide,
4′-cyano-N-[(6S)-6-(dimethylamino)-4-methoxy-5,6,7,8-tetrahydronaphthalen-1-yl]-1,1′-biphenyl-2-sulfonamide,
N-[(6S)-6-(dimethylamino)-4-methoxy-5,6,7,8-tetrahydronaphthalen-1-yl]-1,2-dimethyl-1H-imidazole-4-sulfonamide,
N-[(6S)-6-(dimethylamino)-4-methoxy-5,6,7,8-tetrahydronaphthalen-1-yl]thiophene-3-sulfonamide,
2-chloro-N-[(6S)-6-(dimethylamino)-4-methoxy-5,6,7,8-tetrahydronaphthalen-1-yl]-4,5-difluorobenzenesulfonamide,
N-[(6S)-6-(dimethylamino)-4-methoxy-5,6,7,8-tetrahydronaphthalen-1-yl]-4-(methylsulfonyl)benzenesulfonamide,
4-chloro-N-[(6S)-6-(dimethylamino)-4-methoxy-5,6,7,8-tetrahydronaphthalen-1-yl]-2,5-difluorobenzenesulfonamide,
N-[(6S)-6-(dimethylamino)-4-methoxy-5,6,7,8-tetrahydronaphthalen-1-yl]-1,1′-biphenyl-4-sulfonamide,
N-[(6S)-6-(dimethylamino)-4-methoxy-5,6,7,8-tetrahydronaphthalen-1-yl]-2-methoxy-4-methylbenzenesulfonamide,
N-[(6S)-4-methoxy-6-pyrrolidin-1-yl-5,6,7,8-tetrahydronaphthalen-1-yl]pyridine-3-sulfonamide,
3,5-Dichloro-N-[(6S)-4-methoxy-6-pyrrolidin-1-yl-5,6,7,8-tetrahydronaphthalen-1-yl]benzenesulfonamide,
N-[(6S)-4-Methoxy-6-pyrrolidin-1-yl-5,6,7,8-tetrahydronaphthalen-1-yl]quinoline-8-sulfonamide,
N-[(6S)-4-Methoxy-6-pyrrolidin-1-yl-5,6,7,8-tetrahydronaphthalen-1-yl]naphthalene-1-sulfonamide,
4′-Chloro-N-[(6S)-4-methoxy-6-(methylamino)-5,6,7,8-tetrahydronaphthalen-1-yl]biphenyl-2-sulfonamide,
4′-Chloro-N-[(6S)-4-methoxy-6-(methylamino)-5,6,7,8-tetrahydronaphthalen-1-yl]-N-methylbiphenyl-2-sulfonamide,
N-[(6S)-4-Methoxy-6-pyrrolidin-1-yl-5,6,7,8-tetrahydronaphthalen-1-yl]naphthalene-1-sulfonamide,
5 N-[(6S)-6-(Dimethylamino)-4-methoxy-5,6,7,8-tetrahydronaphthalen-1-yl]quinoline-8-sulfonamide,
4′-Chloro-N-[(6S)-6-(dimethylamino)-4-methoxy-5,6,7,8-tetrahydronaphthalen-1-yl]biphenyl-2-sulfonamide, and
4′-Chloro-N-[(6S)-6-(dimethylamino)-4-methoxy-5,6,7,8-tetrahydronaphthalen-1-yl]-N-methylbiphenyl-2-sulfonamide,
   or salts, solvates or solvated salts thereof.

Listed below are definitions of various terms used in the specification and claims to describe the present invention.

For the avoidance of doubt it is to be understood that where in this specification a group is qualified by ‘hereinbefore defined’, ‘defined hereinbefore’ or ‘defined above’ the said group encompasses the first occurring and broadest definition as well as each and all of the other definitions for that group.

For the avoidance of doubt it is to be understood that in this specification ‘C₁₋₆’ means a carbon group having 1, 2, 3, 4, 5 or 6 carbon atoms.

In this specification, unless stated otherwise, the term “alkyl” includes both straight and branched chain alkyl groups and may be, but are not limited to methyl, ethyl, n-propyl, i-propyl, n-butyl, i-butyl, s-butyl, t-butyl, n-pentyl, i-pentyl, neo-pentyl, n-hexyl or i-hexyl. The term C₁₋₄ alkyl having 1 to 4 carbon atoms and may be but are not limited to methyl, ethyl, n-propyl, i-propyl or tert-butyl.

The term ‘C₀’ means a bond or does not exist. For example “arylCoalkyl” is equivalent with “aryl”, “C₂alkylOC₀alkyl” is equivalent with “C₂alkylO”.

In this specification, unless stated otherwise, the term “alkenyl” includes both straight and branched chain alkenyl groups. The term “C₂₋₆alkenyl” having 2 to 6 carbon atoms and one or two double bonds, may be, but is not limited to vinyl, allyl, propenyl, butenyl, crotyl, pentenyl, or hexenyl, and a butenyl group may for example be buten-2-yl, buten-3-yl or buten-4-yl.

In this specification, unless stated otherwise, the term “alkynyl” includes both straight and branched chain alkynyl groups. The term “C₂₋₆alkynyl” having 2 to 6 carbon atoms and one or two trippel bonds, may be, but is not limited to etynyl, propargyl, pentynyl or hexynyl and a butynyl group may for example be butyn-3-yl or butyn-4-yl.

The term “alkoxy”, unless stated otherwise, refers to radicals of the general formula —O—R, wherein R is selected from a hydrocarbon radical. The term “alkoxy” may include, but is not limited to methoxy, ethoxy, propoxy, isopropoxy, butoxy, t-butoxy, isobutoxy, cyclopropylmethoxy, allyloxy or propargyloxy.

In this specification, unless stated otherwise, the term “amine” or “amino” refers to radicals of the general formula —NRR′, wherein R and R′ are independently selected from hydrogen or a hydrocarbon radical.

In this specification, unless stated otherwise, the term “cycloalkyl” refers to an optionally substituted, completely or partially saturated cyclic hydrocarbon ring system. The term “C₃₋₇cycloalkyl” may be but is not limited to cyclopropyl, cyclobutyl, cyclopentyl, cyclohexyl cycloheptyl or cyclopentenyl.

The term “heterocycloalkyl” denotes a 3- to 7-membered, non-aromatic, partially or completely saturated hydrocarbon group, which contains one ring and at least one heteroatom. Examples of said heterocycle include, but are not limited to pyrrolidinyl, pyrrolidinonyl, piperidinyl, ioxazolyl, (1,3)-thiazolyl, piperazinyl, morpholinyl, oxazolyl, 2-oxazolidonyl or tetrahydrofuranyl.

In this specification, unless stated otherwise, the term “aryl” refers to an optionally substituted monocyclic or bicyclic hydrocarbon ring system with at least one unsaturated aromatic ring. Examples of “aryl” may be, but are not limited to phenyl, naphthyl or tetralinyl.

In this specification, unless stated otherwise, the term “heteroaryl” refers to an optionally substituted monocyclic or bicyclic hydrocarbon ring system with at least one unsaturated aromatic ring and containing at least one heteroatom selected independently form N, O or S. Examples of “heteroaryl” may be, but are not limited to pyridinyl, pyrrolyl, furyl, thienyl, imidazolyl, imidazo[2,1-b][1,3]thiazolyl, 2,1,3-benzoxadiazolyl, benzofurane, quinoline, isoquinoline, oxazolyl, isoxazolyl, benzothiophenyl, thiazolyl, pyrazolyl, benzofuryl, indolyl, isoindolyl, benzimidazolyl, pyridazinyl, pyrimidinyl, pyrazinyl, tetrazolyl, triazolyl, oxazolyl, indolyl, quinazolinyl or chromanyl.

For the avoidance of doubt, a C₅heteroaryl refers to a 5 membered aromatic ring system containing at least one heteroatom.

In this specification, unless stated otherwise, the terms “arylalkyl” and “heteroarylalkyl” refer to a substituent that is attached via the alkyl group to an aryl or heteroaryl group.

In this specification, unless stated otherwise, the terms “halo” and “halogen” may be fluoro, iodo, chloro or bromo.

In this specification, unless stated otherwise, the term “haloalkyl” means an alkyl group as defined above, which is substituted with halo as defined above. The term “C₁₋₆haloalkyl” may include, but is not limited to fluoromethyl, difluoromethyl, trifluoromethyl, fluoroethyl, difluoroethyl or bromopropyl. The term “C₁₋₆haloalkylO” may include, but is not limited to fluoromethoxy, difluoromethoxy, trifluoromethoxy, fluoroethoxy or difluoroethoxy.

The present invention relates to the compounds of formula I as hereinbefore defined as well as to the salts, solvates or solvated salts thereof. Salts for use in pharmaceutical formulations will be pharmaceutically acceptable salts, but other salts may be useful in the production of the compounds of formula I.

A suitable pharmaceutically acceptable salt of the compounds of the invention is, for example, an acid-addition salt, for example a salt with an inorganic or organic acid. In addition, a suitable pharmaceutically acceptable salt of the compounds of the invention is an alkali metal salt, an alkaline earth metal salt or a salt with an organic base. Other pharmaceutically acceptable salts and methods of preparing these salts may be found in, for example, Remington's Pharmaceutical Sciences (18% Edition, Mack Publishing Co.).

Most compounds of formula I may have chiral centres and/or geometric isomeric centres (E- and Z-isomers), and it is to be understood that the invention encompasses all such optical, diastereoisomeric and geometric isomers.

The invention also relates to any and all tautomeric forms of the compounds of formula I.

### Methods of Preparation

One embodiment of the invention relates to processes for the preparation of the compound of formula I wherein R¹ to R¹², p, X, Q and n, unless otherwise specified, are defined as in formula I and PG is a suitable protecting group.

### General Procedure

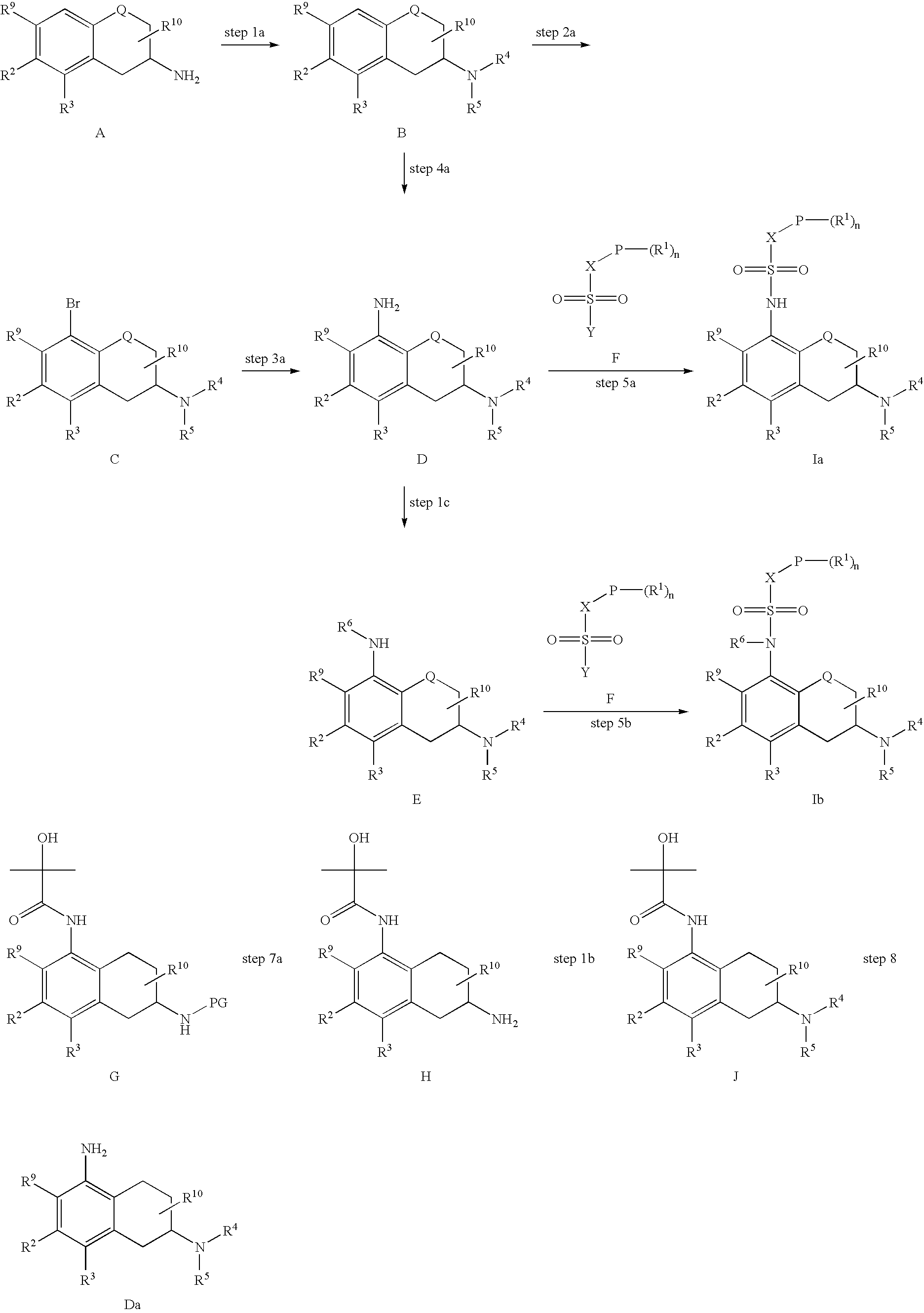

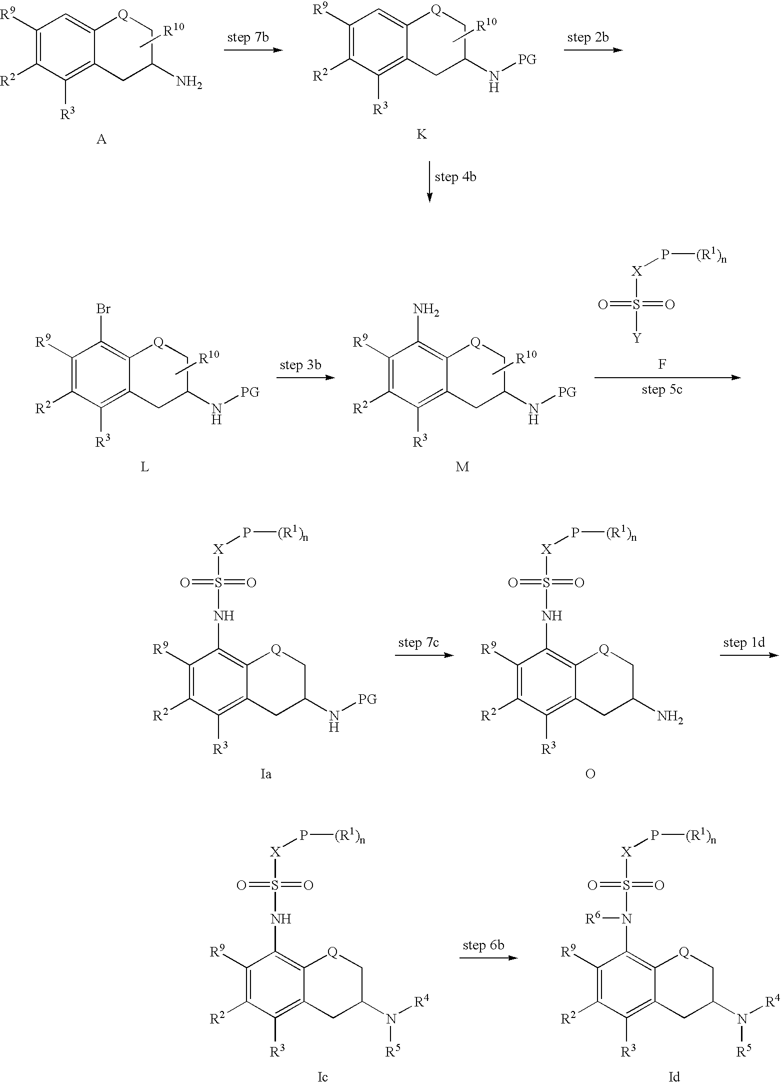

All reactions are run until judged complete by LC-UV, LC-MS or TLC.

### Step 1a, 1b, 1c and 1d

A compound B may be prepared from a compound A by alkylation with a compound R⁴Y or R⁵Y, where Y may be a leaving group such as halogen, mesylate or triflate, as for example described in “Comprehensive Organic Transformations, a Guide to Functional Group Preparation”, R C. Larock, John Wiley & sons, New York, 1999. Typically A and R⁴Y or R⁵Y are mixed in a solvent such as DMF, ethanol, dichloromethane or toluene in the presence of a base such as sodium bicarbonate, sodium carbonate, potassium carbonate, triethylamine or diispropylethylamine and optionally, if Y=Cl, Br, a catalytic amount of potassium iodide or tetrabutylammonium iodide. The reaction may be performed at temperatures between 25° C. and the reflux temperature of the solvent for between 1 hour and 1 week. The reaction mixture may be either worked up by extraction and then purified by column chromatography or the reaction mixture may be concentrated and purified by column chromatography. The reaction temperature may be elevated above the reflux temperature of the solvent and reaction times shortened by the use of microwave heating. For compounds where R⁴ and R⁵ form a ring, a compound YR⁴R⁵Y may be reacted with a compound A.

Alternatively, a compound B may be prepared from a compound A using reductive amination. Typically compound A may be mixed with a carbonyl compound such as an aldehyde or a ketone in the presence of a reducing agent such as sodium borohydride, sodium cyanoborohydride, sodium triacetoxyborohydride or hydrogen, in the presence of a suitable catalyst, as for example described in “Advanced Organic Chemistry—Reactions, Mechanisms and Structure”, J. March, John Wiley & Sons, New York, 1992 or “Comprehensive Organic Transformations, a Guide to Functional Group Preparation”, R C. Larock, John Wiley & sons, New York, 1999. Typically an acid such as formic acid or acetic acid may be added to control the pH of the reaction. The reaction may be performed in a solvent such as water, methanol, ethanol, THF, dichloromethane, formic acid, acetic acid or mixtures thereof at temperatures between 0° C. and the reflux temperature of the solvent, preferably at RT. The reaction mixture may be either worked up by extraction and then purified by column chromatography or the reaction mixture may be concentrated and purified by column chromatography.

A compound B may also be prepared from a compound A by first preparing the amide or carbamate followed by reduction using an appropriate reducing agent. The amide can for example be prepared by reaction of a compound A with an acid chloride with an acid chloride or an acid anhydride optionally in the presence of a base like pyridine, triethylamine or diisopropylethylamine in a solvent like dichloromethane, chloroform or 1-methyl-2-pyrrolidinone. Alternatively, the amide may be prepared by the reaction of A with a carboxylic acid in the presence of a coupling reagent. For methods used in amide formations see for example “Comprehensive Organic Transformations, a Guide to Functional Group Preparation”, R C. Larock, John Wiley & sons, New York. The carbamate may be prepared by the reaction of an alkylchloroformate with a compound A in a solvent such as dichloromethane in the presence of a base such as triethylamine or pyridine at temperatures between 0° C. and the reflux temperature of the solvent. The reduction of the carbamate or the amide may be performed with a reducing agent such as lithium aluminum hydride in a solvent such as tetrahydrofuran or diethyl ether at temperatures between 0° C. and the reflux temperature of the solvent, preferably between 25° C. and the reflux temperature. The reduction of the amide may also be performed using borane as the reducing agent.

The same methods may be used to transform a compound D into a compound E, compound H into a compound J or a compound O into a compound Ic. In step 1c a compound R⁶Y is used instead of a compound R⁴Y or R⁵Y.

### Step 2a and 2b

A compound B may be transformed into a compound C by bromination using bromine in a solvent such as acetic acid, optionally in the presence of sodium acetate. Other solvents that may be used may be for example water, dichloromethane or dioxane. The reaction may be performed at temperatures between 0° C. and the reflux temperature of the solvent, preferably between RT and the reflux temperature. The product may be isolated by precipitation, extraction or column chromatography.

The same method can be used to transform a compound K into a compound L.

### Step 3 a and 3b

A compound C may be transformed into a compound D by a copper mediated amination using aqueous ammonia in a solvent such as DMF in the presence of copper powder. The reaction may be performed at temperatures between 50° C. and the reflux temperature of the solvent, preferably in an autoclave reactor. The product may be isolated by column chromatography, extraction or precipitatation.

Alternatively, a compound C may be transformed into a compound D by a palladium catalyzed coupling with 1,1-diphenylmethanimine followed by hydrolysis. A compound C may be reacted with 1,1-diphenylmethanimine in the presence of a base such as sodium tbutoxide, a ligand such as bis(diphenylphosphino)diphenyl ether and a palladium source such as Pd₂(dba)₃ in a solvent such as toluene, preferably under inert atmosphere at temperatures between 60° C. and the reflux temperature of the solvent. The intermediate imine may be isolated by column chromatography and can then be hydrolyzed to a compound D under acidic conditions using for example aqueous hydrochloric acid in a solvent such as THF at temperatures between 0° C. and the reflux temperature of the solvent, preferably at RT. The product may be isolated by column chromatography, extraction or precipitatation.

The same methods may be used to transform a compound L into a compound M.

### Step 4a and 4b

A compound D may be prepared from a compound B via nitration followed by reduction of the nitrogroup. The nitration may be performed using sodium nitrate in a solvent such as trifluoroacetic acid at temperatures between 0 and 60° C., preferably at room temperature for reaction times between 1 and 10 hours. The nitration may also be performed using nitric acid in a solvent such as sulfuric acid at temperatures between −10° C. and RT. The reduction of the nitro group may be performed using hydrogenation with a suitable catalyst such as palladium on charcoal. For other suitable catalysts or reagents see for example “Comprehensive Organic Transformations, a Guide to Functional Group Preparation”, R C. Larock, John Wiley & sons, New York, 1999.

The same method can be used to transform a compound K into a compound M.

### Step 5a, 5b and 5c

A compound D may be transformed into a compound Ia by reaction with a compound F where Y may be a halogen such as chlorine in a solvent such as DMF, 1-methyl-2-pyrrolidinone, acetonitrile or dichloromethane or mixtures thereof in the presence of a base such as pyridine, triethylamine or DIPEA at temperatures between 0° C. and the reflux temperature of the solvent. The product may be isolated by column chromatography. The same procedure may be used to transform a compound E into a compound 1b or a compound M into a compound N.

### Step 6a and 6b

A compound Ia may be transformed into a compound Ib, where R⁶ is not X, via alkylation with a compound R Y where Y may be a suitable leaving group such as a halogen, mesylate or triflate. The reaction may be performed in the presence of a base such as sodium hydride in an aprotic solvent such as DME or THF at temperatures between 0° C. and the reflux temperature of the solvent. The product may be isolated by column chromatography.

The same method may be used to transform a compound Ic into a compound Id.

### Step 7a, 7b and 7c

A compound G may be transformed into a compound H by protecting group manipulations. Conventional procedures for using such protecting groups, as well as examples of suitable protecting groups are described in, for example, “Protective Groups in Organic Synthesis” T. W. Green, P. G. M. Wuts, Wiley-Interscience, New York, 1999. The same method may be used to transform a compound A into a compound K and a compound Ic into a compound N.

### Step 8,

A compound J may be hydrolyzed of under acidic conditions to form a compound Da using aqueous hydrochloric acid in a solvent such as ethanol or water or a mixture thereof at elevated temperatures such as the reflux temperature of the solvent using reaction times between one and 24 hours. The crude product may be isolated by removal of the solvent or by precipitation or extraction. The product may be purified by column chromatography or recrystallization.

### Intermediates

A further embodiment of the invention relates to compounds selected from the group consisting of

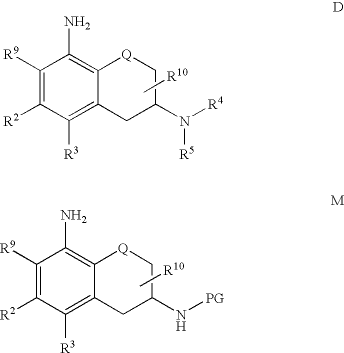

wherein R¹ to R⁹ are defined as hereinbefore and PG is a suitable leaving group, with the proviso that R⁴ and R⁵ are not both n-propyl, and
(3R)-5-methoxy-N³,N³-dimethylchromane-3,8-diamine,
(6S)-4-bromo-N⁶,N⁶-dimethyl-5,6,7,8-tetrahydronaphthalene-1,6-diamine,
(6S)-4-methoxy-N⁶,N⁶-dimethyl-5,6,7,8-tetrahydronaphthalene-1,6-diamine,
(6S)-4-methoxy-6-pyrrolidin-1-yl-5,6,7,8-tetrahydronaphthalen-1-amine, and
N-[(2S)-5-amino-8-methoxy-1,2,3,4-tetrahydronaphthalen-2-yl]-2,2,2-trifluoroacetamide,
   which may be used as intermediates in the preparation of compounds suited for the treatment of 5HT6 mediated disorders, especially for use as intermediates for the preparation of compounds of formula I.

### Pharmaceutical Composition

According to one embodiment of the present invention there is provided a pharmaceutical composition comprising as active ingredient a therapeutically effective amount of the compound of formula I, or salts, solvates or solvated salts thereof, in association with one or more pharmaceutically acceptable diluents, excipients and/or inert carriers.

The composition may be in a form suitable for oral administration, for example as a tablet, pill, syrup, powder, granule or capsule, for parenteral injection (including intravenous, subcutaneous, intramuscular, intravascular or infusion) as a sterile solution, suspension or emulsion, for topical administration e.g. as an ointment, patch or cream, for rectal administration e.g. as a suppository or for inhalation.

In general the above compositions may be prepared in a conventional manner using one or more conventional excipients, pharmaceutical acceptable diluents and/or inert carriers. Suitable daily doses of the compounds of formula I in the treatment of a mammal, including man, are approximately 0.01 to 250 mg/kg bodyweight at peroral administration and about 0.001 to 250 mg/kg bodyweight at parenteral administration.

The typical daily dose of the active ingredient varies within a wide range and will depend on various factors such as the relevant indication, severity of the illness being treated, the route of administration, the age, weight and sex of the patient and the particular compound being used, and may be determined by a physician.

### Medical Use

Interestingly, it has been found that the compounds according to the present invention are useful in therapy. The compounds of formula I, or salts, solvates or solvated salts thereof, as well as their corresponding active metabolites, exhibit a high degree of potency and selectivity for 5-hydroxy-tryptamine 6 (5HT6) receptors. Accordingly, the compounds of the present invention are expected to be useful in the treatment of conditions associated with altered activation of 5HT6 receptors.

The compounds may be used to produce an inhibitory effect of 5HT6 receptors in mammals, including man.

The compounds of formula I are expected to be suitable for the treatment of disorders relating to or affected by the 5HT6 receptor including cognitive, personality, behaviour, psychiatric and neurodegenerative disorders.

Examples of such disorder may be selected from the group comprising of Alzheimer's disease anxiety, depression, convulsive disorders such as epilepsy, personality disorders, obsessive compulsive disorders, migraine, cognitive disorders such as memory dysfunction, sleep disorders, feeding disorders such as anorexia, obesity, bulimia, panic attacks, withdrawal from drug abuse, schizophrenia, attention deficit hyperactive disorder (ADHD), attention deficit disorder (ADD), dementia, memory loss, disorders associated with spinal trauma and/or head injury, stroke, diabetes type 2, binge disorders, bipolar disorders, psychoses, Parkinson's disease, Huntington's disease, neurodegenerative disorders characterized by impaired neuronal growth, and pain.

Further relevant disorders may be selected from the group comprising gastrointestinal disorders such as gastro-esophageal reflux disease (GERD) and irritable bowel syndrome (IBS).

The compounds may also be used for treatment of tolerance to 5HF6 activators.

One embodiment of the invention relates to the compounds of formula I as hereinbefore defined, for use in therapy.

Another embodiment of the invention relates to the compounds of formula I as hereinbefore defined, for use in treatment of 5HT6 mediated disorders.

A further embodiment of the invention relates to the compounds of formula I as hereinbefore defined, for use in treatment of Alzheimer's disease.

Another embodiment of the invention relates to the compounds of formula I as hereinbefore defined, for use in treatment of cognitive impairment associated with schizophrenia.

Yet a further embodiment of the invention relates to the compounds of formula I as hereinbefore defined, for use in treatment of obesity.

One embodiment of the invention relates to the compounds of formula I as hereinbefore defined, for use in Parkinson's disease.

Another embodiment of the invention relates to the use of the compounds of formula I as hereinbefore defined, in the manufacture of a medicament for treatment of 5HT6 mediated disorders, Alzheimer's disease, cognitive impairment associated with schizophrenia, obesity and/or Parkinson's disease, and any other disorder mentioned above.

A further embodiment of the invention relates to a method of treatment of 5HT6 mediated disorders, Alzheimer's disease, cognitive impairment associated with schizophrenia, obesity and/or Parkinson's disease, and any other disorder mentioned above, comprising administering to a mammal, including man in need of such treatment, a therapeutically effective amount of the compounds of formula I, as hereinbefore defined.

Yet another embodiment of the invention relates to a pharmaceutical composition comprising a compound of formula I as hereinbefore defined, for use in treatment of 5HT6 mediated disorders, Alzheimer's disease, cognitive impairment associated with schizophrenia, obesity and/or Parkinson's disease, and any other disorder mentioned above.

One embodiment of the invention relates to an agent for the prevention or treatment of 5HT6 mediated disorders, Alzheimer's disease, cognitive impairment associated with schizophrenia, obesity and/or Parkinson's disease, and any other disorder mentioned above, which comprises as active ingredient a compound of formula I as hereinbefore defined.

In the context of the present specification, the term “therapy” and “treatment” includes prevention and prophylaxis, unless there are specific indications to the contrary. The terms “treat”, “therapeutic” and “therapeutically” should be construed accordingly.

In this specification, unless stated otherwise, the terms “inhibitor” and “antagonist” mean a compound that by any means, partly or completely, blocks the transduction pathway leading to the production of a response by the agonist.

The compounds according to the present invention are modulators of the 5HT6 receptors, and may be inhibitors, as well as agonists, inverse-agonists or partial-agonist.

The term “disorder”, unless stated otherwise, means any condition and disease associated with 5HT6 receptor activity.

### Non-Medical Use

In addition to their use in therapeutic medicine, the compounds of formula I, or salts, solvates or solvated salts thereof, are also useful as pharmacological tools in the development and standardisation of in vitro and in vivo test systems for the evaluation of the effects of modulators of 5HT6 related activity in laboratory animals such as cats, dogs, rabbits, monkeys, rats and mice, as part of the search for new therapeutics agents.

### EXAMPLES

### General Methods

The invention will now be illustrated by the following Examples in which, generally:
(i) operations were carried out at ambient or room temperature, i.e. in the range 17 to 25° C. and under an atmosphere of an inert gas such as argon unless otherwise stated;
(ii) evaporations were carried out by rotary evaporation in vacuo and work-up procedures were carried out after removal of residual solids by filtration;
(iii) HPLC analyses were performed on an Agilent HP1000 system consisting of G1379A Micro Vacuum Degasser, G1312A Binary Pump, G1367A Wellplate auto-sampler, G1316A Thermostatted Column Compartment and G1315B Diode Array Detector. Column: X-Terra MS, Waters, 4.6×50 mm, 3.5 μm. The column temperature was set to 40° C. and the flow rate to 1.5 ml/min. The Diode Array Detector was scanned from 210-300 nm, step and peak width were set to 2 nm and 0.05 min, respectively. A linear gradient was applied, run from 0% to 100% acetonitrile, in 4 min. Mobile phase: acetonitrile/10 mM ammonium acetate in 5% acetonitrile in MilliQ Water.
(iv) Thin layer chromatography (TLC) was performed on Merck TLC-plates (Silica gel 60 F₂₅₄) and UV visualized the spots. Flash chromatography was preformed on a Combi Flash® Companion™ using RediSep™ normal-phase flash columns or on Merck Silica gel 60 (0.040-0.063 mm). Typical solvents used for flash chromatography were mixtures of chloroform/methanol, toluene/ethyl acetate and ethyl acetate/hexanes.
(v) ¹H and ¹³C NMR spectra were recorded at 400 MHz for proton and 100 MHz for carbon-13 either on a Varian Unity+400 NMR Spectrometer equipped with a 5 mm BBO probe with Z-gradients, or a Bruker Avance 400 NMR spectrometer equipped with a 601 dual inverse flow probe with Z-gradients, or a Bruker DPX400 NMR spectrometer equipped with a 4-nucleus probe equipped with Z-gradients. Unless specifically noted in the examples, spectra were recorded at 400 MHz for proton and 100 MHz for carbon-13. The following reference signals were used: the middle line of DMSO-d₆ δ 2.50 (¹H); the middle line of CD₃OD δ 3.31 (¹H); acetone-d₆ 2.04 (¹H); and CDCl₃ δ 7.26 (¹H) (unless otherwise indicated);
(vi) Mass spectra were recorded on a Waters LCMS consisting of an Alliance 2795 (LC) and a ZQ single quadrupole mass spectrometer. The mass spectrometer was equipped with an electrospray ion source (ESI) operated in a positive or negative ion mode. The capillary voltage was 3 kV and the mass spectrometer was scanned from m/z 100-700 with a scan time of 0.3 or 0.8 s. Separations were performed on either Waters X-Terra MS, C8-columns, (3.5 μm, 50 or 100 mm×2.1 mm i.d.), or a ScantecLab's ACE3AQ column (100 mm×2.1 mm i.d.). The column temperature was set to 40° C. A linear gradient was applied using a neutral or acidic mobile phase system, running at 0% to 100% organic phase in 4-5 minutes, flow rate 0.3 ml/min. Mobile phase system: acetonitrile/[10 mM NH₄OAc (aq.)/MeCN (95:5)], or [10 mM NH₄OAc (aq.)/MeCN (1/9)]/[10 mMNH₄OAc(aq.)/MeCN(9/1)]. Acidic mobile phase system: [133 mMHCOOH(aq.)/MeCN(5/95)]/[8 mMHCOOH(aq.)/MeCN(98/2)];
(vii) Alternatively a LC-MS system (Sample Manager 2777C, 1525μ binary pump, 1500 Column Oven, ZQ, PDA2996 and ELS detector, Sedex 85) from Waters was used. Separation was performed using a Zorbax column (C8, 3.0×50 mm, 3 μm). A four minutes linear gradient was used starting at 100% A (A=10 mM NH4OAc in 5% MeOH) and ending at 100% B (MeOH). The ZQ was equipped with a combined APPI/APCI ion source and scanned in the positive mode between m/z 120-800 with a scan time of 0.3 s. The APPI repeller and the APCI corona were set to 0.86 kV and 0.80 μA, respectively. In addition, the desolvation temperature (300° C.), desolvation gas (400 L/Hr) and cone gas (5 L/Hr) were constant for both APCI and APPI mode;
(viii) Preparative chromatography was run on a Gilson auto-preparative HPLC with a diode array detector. Column: XTerra MS C8, 19×300 mm, 7 μm. Gradient with acetonitrile/0.1M ammonium acetate in 5% acetonitrile in MilliQ Water, run from 20% to 60% acetonitrile, in 13 min. Flow rate: 20 ml/min. Alternatively, purification was achieved on a semi preparative Shimadzu LC-8A HPLC with a Shimadzu SPD-10A UV-vis.-detector equipped with a Waters Symmetryo column (C18, 5 μm, 100 mm×19 mm). Gradient with acetonitrile/0.1% trifluoroacetic acid in MilliQ Water, run from 35% to 60% acetonitrile in 20 min. Flow rate: 10 ml/min;
(ix) For the compounds in example 4-167 and 173-311 the following equipment was used: The structure and purity of all intermediates were assessed by HPLC and

NMR analysis. ¹H NMR spectra were determined using a 300 MHz and/or 400 MHz Varian Unity Inova spectrometer with 4-nucleus 5 mm probes installed. LC/MS were performed on Agilent 1100 series HPLC equipped with a 4.6×50 3.5 micron XTerra® MS C8 analytical reverse-phase column (Waters), using a gradient of acetonitrile and a solution of 0.2% 880 ammonia in water at 2 ml/min. Agilent MSD APCI was used for MS detection; both positive and negative ion data were collected when appropriate. All purities of the final products were analysed using a Agilent 1100 series high throughout system, containing:
Agilent 1100 series well plate handler, Agilent 1100 series autointerface, Agilent 1100 series well plate autosampler, 2× Agilent 1100 series binary pumps, Agilent 1100 series thermostated column compartment, Agilent 1100 series diode array detector, Agilent 1100 series mass spectrometer. The stationary phase used was 4.6×20 mm XTerra® MS C₈ IS columns (Waters) analytical reversed-phase column and the mobile phase used was 0.1% 880 ammonia and acetonitrile with UV detection at 220 nm, MS detection with APCI ionisation in positive scan mode. The structures of the final products were confirmed by ¹H NMR spectroscopy recorded using Varian Unity Inova 500 MHz spectrometer, equipped with a 60 ul triple resonance flow probe ant the samples were transferred to the flow cell by direct injection with a Gilson 215 liquids handler. Samples were prepared in 20 ul h6-DMSO+170 ul d6-DMSO to a final concentration of 2.6 mM. h6-DMSO is used for the push solvent. Proton NMR spectra were acquired with WET solvent suppression on both the DMSO and H₂O signals, using Scout-Scan to find the solvent resonances. Spectra were acquired at 25° C.;
(x) All solvents used were analytical grade and commercially available anhydrous solvents for reactions. Reactions were typically run under an inert atmosphere of nitrogen or argon;
(xi) yields, where present, are not necessarily the maximum attainable;
(xii) intermediates were not necessarily fully purified but their structures and purity were assessed by thin layer chromatographic, HPLC, infra-red (IR), MS and/or NMR analysis;
(xiii) melting points are uncorrected and were determined using a Mettler SP62 automatic melting point apparatus or an oil-bath apparatus; melting points for the end-products of the Formula I were determined after crystallisation from an appropriate organic solvent or solvent mixture;
(xiv) the following abbreviations have been used:
   HPLC high performance liquid chromatography
   LC liquid chromatography
   MS mass spectometry
   ret. time retention time
   TFA trifluoroacetic acid
   THF tetrahydrofurane
   DMF dimethylormamide

### DIPEA N,N-diisopropylethylamine

DMSO dimethylsulfoxide
NMP 1-methyl-2-pyrrolidinone
THF tetrahydrofuran
MeOH methanol
RT room temperature
EtOAc Ethyl acetate
LAH lithium aluminumhydride

Throughout the following description of such processes it is understood that, where appropriate, suitable protecting groups will be added to, and subsequently removed from, the various reactants and intermediates in a manner that will be readily understood by one skilled in the art of organic synthesis. The specific sequence of reactions depicted is not critical. For many of the compounds described the order of the reaction steps may be varied.

The invention will now be illustrated by the following non-limiting examples.

Starting Materials were Prepared According to the Following References:

Other starting materials used were either available from commercial sources or prepared according to literature procedures.

Starting materials are either commercially available or prepared according to literature. (3R)-8-Bromo-5-methoxychroman-3-amine was prepared according to WO 9511891, N-[(6S)-6-(Dibenzylamino)-5,6,7,8-tetrahydronaphthalen-1-yl]-2-hydroxy-2-methylpropanamide was prepared according to the procedure described in WO 9734883, [(2S)-8-methoxy-1,2,3,4-tetrahydro-naphthalen-2-yl]-amine (J. Med. Chem. 1989, 32, 779-783).

Other starting materials used were either available from commercial sources or prepared according to literature procedures.

### Example 1

### (i) (3R)-5-Methoxy-N,N-dimethyl-8-[(phenylsulfonyl)amino]chroman-3-ammonium Acetate

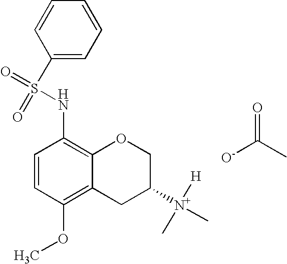

Triethylamine (26 μL, 0.18 mmol) was added to a suspension of (3R)-5-methoxy-N³,N³-dimethylchromane-3,8-diamine (0.06 mmol) in acetonitrile/DMF (0.5 ml:0.1 ml). Benzenesulfonyl chloride (9 μL, 0.066 mmol) was added and the reaction mixture was stirred overnight at room temperature. The product was purified by preparative HPLC to afford the title compound (10 mg, 75%). ¹H NMR (400 MHz, CD₃OD) δ ppm 7.64 (d, 2H), 7.50-7.59 (m, 1H), 7.39-7.50 (m, 2H), 7.20 (d, 1H), 6.49 (d, 1H), 3.72-3.94 (m, 4 H), 3.42-3.55 (m, 1H), 2.74-2.91 (m, 1H), 2.57-2.72 (m, 1H), 2.41-2.56 (m, 1H), 2.35 (s, 6H), 1.94 (s, 3H). MS m/z M+H 363.

### (ii) (3R)-8-Bromo-5-methoxy-N,N-dimethylchroman-3-amine

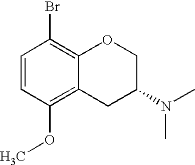

Acetic acid (0.6 ml) was added to a solution of (3R)-8-bromo-5-methoxychroman-3-amine (2.5 g, 9.7 mmol) and formaldehyde (6.7 ml, 80 mmol, 37% solution in H₂O) in MeOH (27 ml) at RT. The solution was cooled to 0° C. and NaCNBH₃ (3.1 g, 50 mmol) was added in two portions. Acetic acid (0.4 ml) was added in order to reach pH 6 and the reaction stirred for one hour. The reaction was allowed to warm up to room temperature and stirred overnight. The solvent was evaporated under reduced pressure, 1 M aqueous NaOH solution was added, and the mixture was extracted with EtOAc (×2). The organic phases were combined, washed with brine, dried over MgSO₄, and the solvent was evaporated under reduced pressure to afford the title compound (1.9 g, 68%). ¹H NMR (400 MHz, CDCl₃) δ ppm 7.30 (d, 1H), 6.35 (d, 1H), 4.45-4.53 (m, 1H), 3.83-3.94 (m, 1H), 3.82 (s, 3H), 2.89-3.00 (m, 1H), 2.51-2.86 (m, 2H), 2.37-2.46 (m, 6H).

MS m/z M+H258.

### (iii) (3R)—N⁸-(Diphenylmethylene)-5-methoxy-N³,N³-dimethylchromane-3,8-diamine

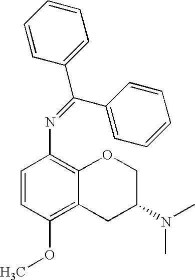

(3R)-8-Bromo-5-methoxy-N,N-dimethylchroman-3-amine (0.57 g, 2 mmol), 1,1-diphenylmethanimine (0.47 g, 2.6 mmol), sodium t-butoxid (0.29 g, 3 mmol), 2,2′-bis(diphenylphosphino)diphenyl ether (65 mg, 0.12 mmol), and Pd₂(dba)₃ were charged into a two-neck round-bottom flask under an argon atmosphere. Anhydrous toluene (8 ml) was added and the reaction mixture heated at 100° C. overnight. The reaction was cooled to room temperature, filtered through Celite and the solvent was evaporated. DMF was added to the residuel and the product was isolated by preparative HPLC. Fractions containing the product were pooled, the acetonitrile was evaporated under reduced pressure, and the aqueous phase was extracted with EtOAc (×2). Organic phases were combined and the solvent was evaporated to afford the title compound (0.35 g, 45%). MS m/z M+H387.6.

### (iv) (3R)-5-Methoxy-N³,N³-dimethylchromane-3,8-diamine, Method A

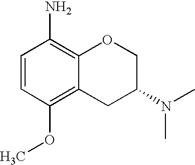

Hydrochloric acid (3 ml, 1M aqueous solution) was added to a solution of (3R)—N⁸-(diphenylmethylene)-5-methoxy-N³,N³-dimethylchromane-3,8-diamine (0.35 g) in THF (10 ml) and the mixture was stirred overnight. Water was added and the solution was washed twice with EtOAc/Heptane (50:50). The aqueous solution was evaporated under reduced pressure and the crude product was used without further purification. ¹H NMR (400 MHz, DMSO-d₆) δ ppm 11.37 (br. s., 1H), 9.90 (br. s., 3H), 7.27 (d, 1H), 6.66 (d, 1H), 4.50-4.59 (m, 1H), 4.29-4.40 (m, 1H), 3.01-3.13 (m, 1H), 2.86-2.97 (m, 1H), 2.77 (s, 6H).

MS m/z: M+H 223.

### Example 2

### (i) (3R)-8-{[(4-Chlorophenyl)sulfonyl]amino}-5-methoxy-N,N-dimethylchroman-3-ammonium Acetate

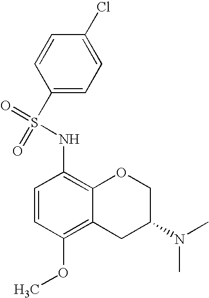

The title compound was synthesized by the analogous preparation of Example 1 (i) and was isolated in 18 mg (52%) yield. ¹H NMR (400 MHz, CD₃OD) δ ppm 7.58-7.63 (m, 2H), 7.44-7.50 (m, 2H), 7.19 (d, 1H), 6.51 (d, 1H), 3.85-3.94 (m, 1H), 3.80 (s, 3H), 3.46-3.57 (m, 1H), 2.76-2.88 (m, 1H), 2.54-2.63 (m, 1H), 2.42-2.53 (m, 1H), 2.37 (s, 6H), 1.95 (s, 3H). MS m/z M−H 395, M+H 397.

### Example 3

### (i) 3-Bromo-N-[(3R)-3-(dimethylamino)-5-methoxy-3,4-dihydro-2H-chromen-8-yl]benzenesulfonamide

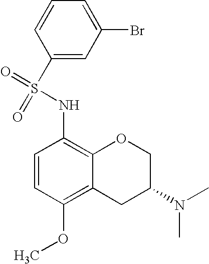

The title compound was synthesized by the analogous preparation of Example 1 (i) and was isolated in 23 mg (58%) yield. ¹H NMR (400 MHz, CD₃OD) δ ppm 7.76 (t, 1H), 7.68-7.73 (m, 1H), 7.56-7.62 (m, 1H), 6.36 (t, 1H), 7.19 (d, 1H), 7.50 (d, 1H), 3.85-3.94 (m, 1H), 3.80 (s, 3H), 3.40-3.50 (m, 1H), 2.76-2.88 (m, 1H), 2.48-2.55 (m, 1H), 2.39-2.48 (m, 1H), 2.33 (s, 6H), 1.5 (s, 3H). MS m/z M-1 439, 441, M+H 441, 443.

### Example 4

### (i) N-[(3R)-3-(Dimethylamino)-5-methoxy-3,4-dihydro-2H-chromen-8-yl]biphenyl-4-sulfonamide

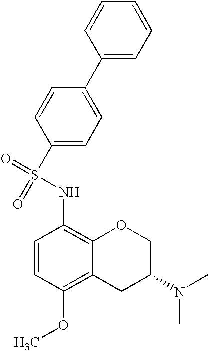

The title compound was synthesized by the analogous preparation of Example 1 (i) and was isolated in 14 mg (36%) yield. ¹H NMR (400 MHz, CD₃OD) δ ppm 7.58-7.75 (m, 6H), 7.46 (t, 2H), 7.35-7.43 (m, 1H), 7.22 (d, 1H), 6.50 (d, 1H), 3.83-3.92 (m, 1H), 3.80 (s, 3H), 3.24-3.30 (m, 1H), 2.67-2.81 (m, 1H), 2.27-2.43 (m, 2H), 2.16 (s, 6H).

MS m/z M+H 439, M−H 437.

### Example 5

### (i) N-[(3R)-3-(dimethylamino)-5-methoxy-3,4-dihydro-2H-chromen-8-yl]-2-methoxy-4-methylbenzenesulfonamide

To a solution of 2-methoxy-4-methylbenzenesulfonyl chloride (22 mg, 0.10 mmol) in N-methylpyrrolidine (200 μL) was added a solution of (3R)-5-methoxy-N³,N³-dimethylchromane-3,8-diamine (22 mg, 0.10 mmol) in N-methylpyrrolidine (200 μL) and triethylamine (42 μL, 0.30 mmol). The reaction mixture was shaken for 18 hours at room temperature and the volatiles were removed under vacuum. The crude product was purified first using polymer supported tosic(65) resin, loading as a solution in methanol (500 ΔL) followed by washing with excess methanol (2.0 ml) and finally eluting with 1M ammonia solution in methanol (1.0 ml). The methanol was removed under vacuum and the residue was further purified using reversed phase preparative HPLC to give the named product (19.7 mg). ¹H NMR (500 MHz, DMSO-d₆) δ 8.21 (s, 1H), 7.40 (d, 1H), 7.03 (s, 1H), 6.97 (d, 1H), 6.76 (d, 1H), 6.40 (d, 1H), 4.06 (d, 1H), 3.90 (s, 3H), 3.70 (s, 3H), 3.52-3.46 (m, 1H), 2.86-2.82 (m, 1H), 2.55-2.49 (m, 1H), 2.40-2.35 (m, 1H), 2.33 (s, 3H), 2.18 (s, 6H). MS m/z (APCI+) M+H407.

### (ii) (3R)-5-methoxy-N³,N³-dimethylchromane-3,8-diamine, Method B

To a solution of [(3R)-8-bromo-5-methoxy-3,4-dihydro-2H-chromen-3-yl]dimethylamine (4.00 g, 14.0 mmol) (Example 1 (ii)) in dimethylformamide (20.0 ml) in an autoclave container was added a concentrated aqueous ammonia solution (20 ml) and copper powder (1.06 g, 16.7 mmol). The container was then sealed and the reaction was heated to 110° C. for 18 hours with stirring. After it has cooled to RT, the reaction mixture was poured into saturated ammonium chloride solution (30 ml) and the aqueous layer was extracted with dichloromethane (3×50 ml). The combined organic layers were washed with a saturated ammonium chloride solution (100 ml) followed by a saturated sodium chloride solution (100 ml) and was dried over sodium sulphate, filtered and concentrated in vacuo to give an oil (3.05 g). The presence of the title compound was confirmed by LC/MS (purity >95%) and the crude material was used immediately in the next step. ¹H NMR (400 MHz, DMSO-d₆) δ 6.41 (d, 1H), 6.26 (d, 1H), 4.31-4.27 (m, 1H), 4.17-4.07 (m, 2H), 3.72 (t, 1H), 3.65 (s, 3H), 2.75 (ddd, 1H), 2.57-2.51 (m, 1H), 2.45-2.39 (m, 1H), 2.26 (s, 6H). MS m/z (APCI+) M+H 223.

### Example 6 to 24

The following compounds were synthesized in an analogous method to Example 5 (i)

| | | MS | NMR Data ¹H NMR (500 MHz, |
|---|---|---|---|
| Example | Name | (M + H)⁺ | DMSO-d₆), δ |
| 5 | N-[(3R)-3- | 407 | 8.21 (s, 1H), 7.40 (d, 1H), 7.03 (s, 1H), |
| | (dimethylamino)-5- | | 6.97 (d, 1H), 6.76 (d, 1H), 6.40 (d, 1H), |
| | methoxy-3,4- | | 4.06 (d, 1H), 3.90 (s, 3H), 3.70 (s, 3H), |
| | dihydro-2H- | | 3.52-3.46 (m, 1H), 2.86-2.82 (m, 1H), |
| | chromen-8-yl]-2- | | 2.55-2.49 (m, 1H), 2.40-2.35 (m, 1H), |
| | methoxy-4- | | 2.33 (s, 3H), 2.18 (s, 6H). |
| | methylbenzenesulfonamide | | |
| 7 | 6-chloro-N-[(3R)-3- | 443 | 7.80 (d, 1H), 7.59 (d, 1H), 7.05 (d, 1H), |
| | (dimethylamino)-5- | | 6.51 (d, 1H), 3.75 (s, 3H), 3.64 (d, 1H), |
| | methoxy-3,4- | | 3.55-3.51 (m, 1H), 3.13 (t, 1H), |
| | dihydro-2H- | | 2.54-2.48 (m, 1H), 2.29-2.25 (m, 1H), |
| | chromen-8- | | 2.14 (s, 6H). |
| | yl]imidazo[2,1- | | |
| | b][1,3]thiazole-5- | | |
| | sulfonamide | | |
| 8 | N-[(3R)-3- | 441 | 8.28 (t, 1H), 7.93 (d, 1H), 7.81 (t, 1H), |
| | (dimethylamino)-5- | | 7.75 (d, 1H), 7.10 (d, 1H), 6.51 (d, 1H), |
| | methoxy-3,4- | | 3.74 (s, 3H), 3.64 (d, 1H), 3.47-3.44 (m, |
| | dihydro-2H- | | 1H), 3.18 (t, 1H), 2.89 (s, 3H), |
| | chromen-8-yl]-2- | | 2.58-2.50 (m, 1H), 2.30-2.24 (m, 1H), 2.09 (s, |
| | (methylsulfonyl)benzenesulfonamide | | 6H). |
| 9 | 5-chloro-N-[(3R)-3- | 467 | 8.04 (d, 1H), 8.03 (d, 1H), 8.01 (s, 1H), |
| | (dimethylamino)-5- | | 7.55 (d, 1H), 7.04 (d, 1H), 3.75 (s, 3H), |
| | methoxy-3,4- | | 3.74-3.70 (m, 1H), 3.58-3.53 (m, 1H), |
| | dihydro-2H- | | 3.08-3.02 (m, 1H), 2.57-2.50 (m, 1H), |
| | chromen-8-yl]-3- | | 2.36 (s, 3H), 2.22-2.14 (m, 1H), 1.95 (s, |
| | methyl-1- | | 6H). |
| | benzothiophene-2- | | |
| | sulfonamide | | |
| 10 | 7-chloro-N-[(3R)-3- | 439 | 7.82 (d, 1H), 7.67 (d, 1H), 7.04 (d, 1H), |
| | (dimethylamino)-5- | | 6.51 (d, 1H), 3.75 (s, 3H), 3.64 (d, 1H), |
| | methoxy-3,4- | | 3.27 (m, 1H), 3.06-3.02 (m, 1H), |
| | dihydro-2H- | | 2.58-2.50 (m, 1H), 2.30-2.21 (m, 1H), |
| | chromen-8-yl]-2,1,3- | | 2.07 (s, 6H). |
| | benzoxadiazole-4- | | |
| | sulfonamide | | |
| 11 | N-[(3R)-3- | 447 | 9.50 (s, 1H), 7.68-7.60 (m, 3H), 7.50 (s, |
| | (dimethylamino)-5- | | 1H), 7.01 (d, 1H), 6.50 (d, 1H), |
| | methoxy-3,4- | | 3.81-3.76 (m, 1H), 3.75 (s, 3H), |
| | dihydro-2H- | | 3.36-3.30 (m, 1H), 3.22-3.17 (m, 1H), 2.59, |
| | chromen-8-yl]-3- | | 2.49 (m, 1H), 2.33-2.25 (m, 1H), 2.15 (s, 6H) |
| | (trifluoromethoxy)benzenesulfonamide | | |
| 12 | N-[(3R)-3- | 421 | 9.06 (s, 1H), 7.09-7.06 (m, 2H), 6.95 (d, |
| | (dimethylamino)-5- | | 1H), 6.46 (d, 1H), 4.31-4.24 (m, 4H), |
| | methoxy-3,4- | | 3.94-3.91 (m, 2H), 3.74 (s, 3H), |
| | dihydro-2H- | | 3.36-3.31 (m, 1H), 2.74-2.64 (m, 1H), |
| | chromen-8-yl]-2,3- | | 2.58-2.50 (m, 1H), 2.42-2.33 (m, 1H), |
| | dihydro-1,4- | | 2.19 (s, 6H). |
| | benzodioxine-6- | | |
| | sulfonamide | | |
| 13 | 3-(2- | 489 | 9.29 (s, 1H), 7.63 (d, 1H), 7.53 (t, 1H), |
| | chlorophenoxy)-N- | | 7.40-7.28 (m, 3H), 7.21 (d, 1H), 7.11 (d, |
| | [(3R)-3- | | 1H), 6.99 (s, 1H), 6.94 (d, 1H), 6.45 (d, |
| | (dimethylamino)-5- | | 1H), 3.87 (d, 1H), 3.76 (s, 3H), |
| | methoxy-3,4- | | 3.37-3.35 (m, 1H), 2.80-2.74 (m, 1H), |
| | dihydro-2H- | | 2.57-2.50 (m, 1H), 2.36-2.31 (m, 1H), |
| | chromen-8- | | 2.17 (s, 6H). |
| | yl]benzenesulfonamide | | |
| 14 | 4,5-dichloro-N- | 437 | 7.36 (s, 1H), 7.01 (d, 1H), 6.53 (d, 1H), |
| | [(3R)-3- | | 3.96 (d, 1H), 3.78 (s, 3H), 3.53-3.47 (m, |
| | (dimethylamino)-5- | | 1H), 2.90-2.88 (m, 1H), 2.59-2.54 (m, |
| | methoxy-3,4- | | 1H), 2.39-2.35 (m, 1H), 2.21 (s, 6H) |
| | dihydro-2H- | | |
| | chromen-8- | | |
| | yl]thiophene-2- | | |
| | sulfonamide | | |
| 15 | N-[(3R)-3- | 441 | 8.99 (s, 1H), 7.83 (d, 1H), 7.58-7.53 (m, |
| | (dimethylamino)-5- | | 3H), 7.46-7.40 (m, 3H), 7.09 (d, 1H), |
| | methoxy-3,4- | | 6.53 (d, 1H), 4.08 (d, 1H), 3.77 (s, 3H), |
| | dihydro-2H- | | 3.69 (t, 2H), 3.51 (t, 2H), 3.37-3.31 (m, |
| | chromen-8-yl]-2-(1- | | 1H), 2.72-2.66 (m, 1H), 2.58-2.49 (m, |
| | naphthyl)ethanesulfonamide | | 1H), 2.45-2.37 (m, 1H), 2.09 (s, 6H). |
| 16 | 4-chloro-N-[(3R)-3- | 447 | 9.60 (s, 1H), 8.76 (d, 1H), 8.34 (d, 1H), |
| | (dimethylamino)-5- | | 7.86-7.80 (m, 2H), 7.77-7.72 (m, 2H), |
| | methoxy-3,4- | | 7.00 (d, 1H), 6.46 (d, 1H), 3.71 (s, 3H), |
| | dihydro-2H- | | 3.36-3.31 (m, 1H), 2.72-2.62 (m, 1H), |
| | chromen-8- | | 2.58-2.50 (m, 1H), 2.13-2.07 (m, 1H), |
| | yl]naphthalene-1- | | 1.99 (s, 6H), 1.87-1.78 (m, 1H). |
| | sulfonamide | | |
| 17 | 4′-cyano-N-[(3R)-3- | 464 | 8.89 (s, 1H), 7.79 (d, 3H), 7.65 (t, 1H), |
| | (dimethylamino)-5- | | 7.55 (t, 1H), 7.41 (d, 2H), 7.29 (d, 1H), |
| | methoxy-3,4- | | 6.86 (d, 1H), 6.44 (d, 1H), 3.89 (d, 1H), |
| | dihydro-2H- | | 3.76 (s, 3H), 2.39-3.33 (m, 1H), |
| | chromen-8-yl]-1,1′- | | 2.79-2.74 (m, 1H), 2.57-2.49 (m, 1H), |
| | biphenyl-2- | | 2.40-2.34 (m, 1H), 2.17 (s, 6H). |
| | sulfonamide | | |
| 18 | N-[(3R)-3- | 431 | 9.57 (s, 1H), 8.01 (d, 1H), 7.90 (s, 1H), |
| | (dimethylamino)-5- | | 7.88-7.85 (m, 1H), 7.79-7.75 (m, 1H), |
| | methoxy-3,4- | | 7.03 (d, 1H), 6.51 (d, 1H), 3.75 (s, 3H), |
| | dihydro-2H- | | 3.73-3.68 (m, 1H), 3.37-3.32 (m, 1H), |
| | chromen-8-yl]-3- | | 3.16 (t, 1H), 2.57-2.49 (m, 1H), |
| | (trifluoromethyl)benzenesulfonamide | | 2.31-2.24 (m, 1H), 2.13 (s, 6H). |
| 19 | N-[(3R)-3- | 446 | 8.55 (s, 1H), 7.89 (t, 1H), 7.77 (s, 1H), |
| | (dimethylamino)-5- | | 7.37 (t, 1H), 7.34 (s, 1H), 7.02 (d, 1H), |
| | methoxy-3,4- | | 6.51 (d, 1H), 6.42 (d, 1H), 6.27 (d, 1H), |
| | dihydro-2H- | | 4.29 (d, 1H), 3.91-3.86 (m, 1H), 3.76 (s, |
| | chromen-8-yl]-5- | | 3H), 3.74-3.69 (m, 1H), 2.58-2.50 (m, |
| | pyridin-2- | | 1H), 2.36-2.28 (m, 1H), 2.05 (s, 6H). |
| | ylthiophene-2- | | |
| | sulfonamide | | |
| 20 | N-[3-({[(3R)-3- | 420 | 10.19 (s, 1H), 9.18 (s, 1H), 7.92 (s, 1H), |
| | (dimethylamino)-5- | | 7.76 (d, 1H), 7.42 (t, 1H), 7.26 (d, 1H), |
| | methoxy-3,4- | | 6.94 (d, 1H), 6.46 (d, 1H), 3.84 (d, 1H), |
| | dihydro-2H- | | 3.74 (s, 3H), 3.36-3.30 (m, 1H), |
| | chromen-8- | | 2.71-2.64 (m, 1H), 2.59-2.51 (m, 1H), |
| | yl]amino}sulfonyl)phenyl]- | | 2.35-2.28 (m, 1H), 2.04 (s, 6H). |
| | acetamide | | |
| 21 | 1-acetyl-5-bromo-N- | 524 | 9.00 (s, 1H), 8.53 (s, 1H), 7.68 (s, 1H), |
| | [(3R)-3- | | 6.89 (d, 1H), 6.42 (d, 1H), 4.14-4.08 (m, |
| | (dimethylamino)-5- | | 2H), 4.01-3.97 (m, 1H), 3.72 (s, 3H), |
| | methoxy-3,4- | | 3.38-3.31 (m, 1H), 3.20-3.15 (m, 2H), |
| | dihydro-2H- | | 2.70-2.64 (m, 1H), 2.52-2.46 (m, 1H), |
| | chromen-8- | | 2.42-2.35 (m, 1H), 2.19 (s, 3H), 2.14 (s, |
| | yl]indoline-6- | | 6H) |
| | sulfonamide | | |
| 22 | 4-cyano-N-[(3R)-3- | 388 | 8.02 (d, 2H), 7.75 (d, 2H), 6.99 (d, 1H), |
| | (dimethylamino)-5- | | 6.49 (d, 1H), 3.75 (s, 3H), 3.74-3.71 (m, |
| | methoxy-3,4- | | 1H), 3.26-3.21 (m, 1H), 2.80-2.74 (m, |
| | dihydro-2H- | | 1H), 2.58-2.50 (m, 1H), 2.35-2.28 (m, |
| | chromen-8- | | 1H), 2.16 (s, 6H). |
| | yl]benzenesulfonamide | | |
| 23 | N-[(3R)-3- | 405 | 9.10 (s, 1H), 7.51 (d, 2H), 7.32 (d, 2H), |
| | (dimethylamino)-5- | | 6.98 (d, 1H), 6.47 (d, 1H), 3.84-3.79 (m, |
| | methoxy-3,4- | | 1H), 3.74 (s, 3H), 3.24-3.16 (m, 1H), |
| | dihydro-2H- | | 2.75-2.67 (m, 1H), 2.57-2.48 (m, 1H), |
| | chromen-8-yl]-4- | | 2.32-2.26 (m, 1H), 2.16 (s, 6H), |
| | propylbenzenesulfonamide | | 1.62-1.55 (m, 2H), 1.17 (t, 3H). |
| | amide | | |
| 24 | N-[(3R)-3- | 413 | 9.30 (s, 1H), 8.17 (s, 1H), 8.08-8.05 (m, |
| | (dimethylamino)-5- | | 2H), 8.02 (d, 1H), 7.72 (d, 1H), 7.68 (t, |
| | methoxy-3,4- | | 1H), 7.63 (t, 1H), 7.00 (d, 1H), 6.46 (d, |
| | dihydro-2H- | | 1H), 3.72 (s, 3H), 3.58 (d, 1H), 3.05 (t, |
| | chromen-8- | | 1H), 2.71-2.65 (m, 1H), 2.58-2.50 (m, |
| | yl]naphthalene-2- | | 1H), 2.19 (dd, 1H), 1.98 (s, 6H). |
| | sulfonamide | | |

### Example 25 to 167

The following compounds were synthesized in an analogous method to Example 5 (i)

| Example | MS (M + H)⁺ | Name |
|---|---|---|
| 25 | 377 | N-[(3R)-3-(dimethylamino)-5-methoxy- |
| | | 3,4-dihydro-2H-chromen-8-yl]-4- |
| | | methylbenzenesulfonamide |
| 26 | 441 | 4-bromo-N-[(3R)-3-(dimethylamino)-5- |
| | | methoxy-3,4-dihydro-2H-chromen-8- |
| | | yl]benzenesulfonamide |
| 27 | 481 | 3-bromo-5-chloro-N-[(3R)-3- |
| | | (dimethylamino)-5-methoxy-3,4-dihydro- |
| | | 2H-chromen-8-yl]thiophene-2- |
| | | sulfonamide |
| 28 | 419 | 4-tert-butyl-N-[(3R)-3-(dimethylamino)-5- |
| | | methoxy-3,4-dihydro-2H-chromen-8- |
| | | yl]benzenesulfonamide |
| 29 | 393 | N-[(3R)-3-(dimethylamino)-5-methoxy- |
| | | 3,4-dihydro-2H-chromen-8-yl]-4- |
| | | methoxybenzenesulfonamide |
| 30 | 397 | 2-chloro-N-[(3R)-3-(dimethylamino)-5- |
| | | methoxy-3,4-dihydro-2H-chromen-8- |
| | | yl]benzenesulfonamide |
| 31 | 420 | N-[4-({[(3R)-3-(dimethylamino)-5- |
| | | methoxy-3,4-dihydro-2H-chromen-8- |
| | | yl]amino}sulfonyl)phenyl]acetamide |
| 32 | 441 | 2-bromo-N-[(3R)-3-(dimethylamino)-5- |
| | | methoxy-3,4-dihydro-2H-chromen-8- |
| | | yl]benzenesulfonamide |
| 33 | 502 | N-{[5-({[(3R)-3-(dimethylamino)-5- |
| | | methoxy-3,4-dihydro-2H-chromen-8- |
| | | yl]amino}sulfonyl)thien-2- |
| | | yl]methyl}benzamide |
| 34 | 431 | N-[(3R)-3-(dimethylamino)-5-methoxy- |
| | | 3,4-dihydro-2H-chromen-8-yl]-4- |
| | | (trifluoromethyl)benzenesulfonamide |
| 35 | 391 | N-[(3R)-3-(dimethylamino)-5-methoxy- |
| | | 3,4-dihydro-2H-chromen-8-yl]-4- |
| | | ethylbenzenesulfonamide |
| 36 | 408 | N-[(3R)-3-(dimethylamino)-5-methoxy- |
| | | 3,4-dihydro-2H-chromen-8-yl]-2- |
| | | nitrobenzenesulfonamide |
| 37 | 465 | 2-chloro-N-[(3R)-3-(dimethylamino)-5- |
| | | methoxy-3,4-dihydro-2H-chromen-8-yl]- |
| | | 5-(trifluoromethyl)benzenesulfonamide |
| 38 | 422 | N-[(3R)-3-(dimethylamino)-5-methoxy- |
| | | 3,4-dihydro-2H-chromen-8-yl]-4-methyl- |
| | | 3-nitrobenzenesulfonamide |
| 39 | 413 | N-[(3R)-3-(dimethylamino)-5-methoxy- |
| | | 3,4-dihydro-2H-chromen-8- |
| | | yl]naphthalene-1-sulfonamide |
| 40 | 442 | 4-chloro-N-[(3R)-3-(dimethylamino)-5- |
| | | methoxy-3,4-dihydro-2H-chromen-8-yl]- |
| | | 3-nitrobenzenesulfonamide |
| 41 | 397 | 4-chloro-N-[(3R)-3-(dimethylamino)-5- |
| | | methoxy-3,4-dihydro-2H-chromen-8- |
| | | yl]benzenesulfonamide |
| 42 | 431 | 2,4-dichloro-N-[(3R)-3-(dimethylamino)- |
| | | 5-methoxy-3,4-dihydro-2H-chromen-8- |
| | | yl]benzenesulfonamide |
| 43 | 441 | N-[5-({[(3R)-3-(dimethylamino)-5- |
| | | methoxy-3,4-dihydro-2H-chromen-8- |
| | | yl]amino}sulfonyl)-4-methyl-1,3-thiazol- |
| | | 2-yl]acetamide |
| 44 | 369 | N-[(3R)-3-(dimethylamino)-5-methoxy- |
| | | 3,4-dihydro-2H-chromen-8-yl]thiophene- |
| | | 2-sulfonamide |
| 45 | 408 | N-[(3R)-3-(dimethylamino)-5-methoxy- |
| | | 3,4-dihydro-2H-chromen-8-yl]-4- |
| | | nitrobenzenesulfonamide |
| 46 | 447 | 3,5-dichloro-N-[(3R)-3-(dimethylamino)- |
| | | 5-methoxy-3,4-dihydro-2H-chromen-8- |
| | | yl]-2-hydroxybenzenesulfonamide |
| 47 | 408 | N-[(3R)-3-(dimethylamino)-5-methoxy- |
| | | 3,4-dihydro-2H-chromen-8-yl]-3- |
| | | nitrobenzenesulfonamide |
| 48 | 423 | N-[(3R)-3-(dimethylamino)-5-methoxy- |
| | | 3,4-dihydro-2H-chromen-8-yl]-2,5- |
| | | dimethoxybenzenesulfonamide |
| 49 | 525 | 4,5-dibromo-N-[(3R)-3-(dimethylamino)- |
| | | 5-methoxy-3,4-dihydro-2H-chromen-8- |
| | | yl]thiophene-2-sulfonamide |
| 50 | 471 | 5-bromo-N-[(3R)-3-(dimethylamino)-5- |
| | | methoxy-3,4-dihydro-2H-chromen-8-yl]- |
| | | 2-methoxybenzenesulfonamide |
| 51 | 509 | N-[(3R)-3-(dimethylamino)-5-methoxy- |
| | | 3,4-dihydro-2H-chromen-8-yl]-5- |
| | | (phenylsulfonyl)thiophene-2-sulfonamide |
| 52 | 517 | N-[(3R)-3-(dimethylamino)-5-methoxy- |
| | | 3,4-dihydro-2H-chromen-8-yl]-5-[1- |
| | | methyl-5-(trifluoromethyl)-1H-pyrazol-3- |
| | | yl]thiophene-2-sulfonamide |
| 53 | 388 | 2-cyano-N-[(3R)-3-(dimethylamino)-5- |
| | | methoxy-3,4-dihydro-2H-chromen-8- |
| | | yl]benzenesulfonamide |
| 54 | 415 | 5-chloro-N-[(3R)-3-(dimethylamino)-5- |
| | | methoxy-3,4-dihydro-2H-chromen-8-yl]- |
| | | 1,3-dimethyl-1H-pyrazole-4-sulfonamide |
| 55 | 363 | N-[(3R)-3-(dimethylamino)-5-methoxy- |
| | | 3,4-dihydro-2H-chromen-8- |
| | | yl]benzenesulfonamide |
| 56 | 382 | N-[(3R)-3-(dimethylamino)-5-methoxy- |
| | | 3,4-dihydro-2H-chromen-8-yl]-3,5- |
| | | dimethylisoxazole-4-sulfonamide |
| 57 | 367 | N-[(3R)-3-(dimethylamino)-5-methoxy- |
| | | 3,4-dihydro-2H-chromen-8-yl]-1-methyl- |
| | | 1H-imidazole-4-sulfonamide |
| 58 | 436 | N-[(3R)-3-(dimethylamino)-5-methoxy- |
| | | 3,4-dihydro-2H-chromen-8-yl]-5-isoxazol- |
| | | 3-ylthiophene-2-sulfonamide |
| 59 | 427 | methyl 3-({[(3R)-3-(dimethylamino)-5- |
| | | methoxy-3,4-dihydro-2H-chromen-8- |
| | | yl]amino}sulfonyl)thiophene-2- |
| | | carboxylate |
| 60 | 455 | N-[(3R)-3-(dimethylamino)-5-methoxy- |
| | | 3,4-dihydro-2H-chromen-8-yl]-4- |
| | | phenoxybenzenesulfonamide |
| 61 | 499 | N-[(3R)-3-(dimethylamino)-5-methoxy- |
| | | 3,4-dihydro-2H-chromen-8-yl]-3,5- |
| | | bis(trifluoromethyl)benzenesulfonamide |
| 62 | 431 | 2,6-dichloro-N-[(3R)-3-(dimethylamino)- |
| | | 5-methoxy-3,4-dihydro-2H-chromen-8- |
| | | yl]benzenesulfonamide |
| 63 | 399 | N-[(3R)-3-(dimethylamino)-5-methoxy- |
| | | 3,4-dihydro-2H-chromen-8-yl]-2,6- |
| | | difluorobenzenesulfonamide |
| 64 | 422 | N-[(3R)-3-(dimethylamino)-5-methoxy- |
| | | 3,4-dihydro-2H-chromen-8-yl]-2-methyl- |
| | | 5-nitrobenzenesulfonamide |
| 65 | 433 | N-[(3R)-3-(dimethylamino)-5-methoxy- |
| | | 3,4-dihydro-2H-chromen-8-yl]-4-tert- |
| | | pentylbenzenesulfonamide |
| 66 | 453 | N-[(3R)-3-(dimethylamino)-5-methoxy- |
| | | 3,4-dihydro-2H-chromen-8-yl]-3,4,5- |
| | | trimethoxybenzenesulfonamide |
| 67 | 377 | N-[(3R)-3-(dimethylamino)-5-methoxy- |
| | | 3,4-dihydro-2H-chromen-8-yl]-3- |
| | | methylbenzenesulfonamide |
| 68 | 447 | N-[(3R)-3-(dimethylamino)-5-methoxy- |
| | | 3,4-dihydro-2H-chromen-8-yl]-2- |
| | | (trifluoromethoxy)benzenesulfonamide |
| 69 | 459 | 4-bromo-N-[(3R)-3-(dimethylamino)-5- |
| | | methoxy-3,4-dihydro-2H-chromen-8-yl]- |
| | | 2-fluorobenzenesulfonamide |
| 70 | 422 | N-[(3R)-3-(dimethylamino)-5-methoxy- |
| | | 3,4-dihydro-2H-chromen-8-yl]-2-methyl- |
| | | 4-nitrobenzenesulfonamide |
| 71 | 397 | 3-chloro-N-[(3R)-3-(dimethylamino)-5- |
| | | methoxy-3,4-dihydro-2H-chromen-8- |
| | | yl]benzenesulfonamide |
| 72 | 415 | 2-chloro-N-[(3R)-3-(dimethylamino)-5- |
| | | methoxy-3,4-dihydro-2H-chromen-8-yl]- |
| | | 4-fluorobenzenesulfonamide |
| 73 | 415 | 3-chloro-N-[(3R)-3-(dimethylamino)-5- |
| | | methoxy-3,4-dihydro-2H-chromen-8-yl]- |
| | | 4-fluorobenzenesulfonamide |
| 74 | 465 | 2-chloro-N-[(3R)-3-(dimethylamino)-5- |
| | | methoxy-3,4-dihydro-2H-chromen-8-yl]- |
| | | 4-(trifluoromethyl)benzenesulfonamide |
| 75 | 377 | N-[(3R)-3-(dimethylamino)-5-methoxy- |
| | | 3,4-dihydro-2H-chromen-8-yl]-1- |
| | | phenylmethanesulfonamide |
| 76 | 399 | N-[(3R)-3-(dimethylamino)-5-methoxy- |
| | | 3,4-dihydro-2H-chromen-8-yl]-2,4- |
| | | difluorobenzenesulfonamide |
| 77 | 395 | N-[(3R)-3-(dimethylamino)-5-methoxy- |
| | | 3,4-dihydro-2H-chromen-8-yl]-5-fluoro-2- |
| | | methylbenzenesulfonamide |
| 78 | 447 | N-[(3R)-3-(dimethylamino)-5-methoxy- |
| | | 3,4-dihydro-2H-chromen-8-yl]-4- |
| | | (trifluoromethoxy)benzenesulfonamide |
| 79 | 431 | 2,5-dichloro-N-[(3R)-3-(dimethylamino)- |
| | | 5-methoxy-3,4-dihydro-2H-chromen-8- |
| | | yl]benzenesulfonamide |
| 80 | 465 | 2,4,6-trichloro-N-[(3R)-3- |
| | | (dimethylamino)-5-methoxy-3,4-dihydro- |
| | | 2H-chromen-8-yl]benzenesulfonamide |
| 81 | 441 | 3-bromo-N-[(3R)-3-(dimethylamino)-5- |
| | | methoxy-3,4-dihydro-2H-chromen-8- |
| | | yl]benzenesulfonamide |
| 82 | 411 | 3-chloro-N-[(3R)-3-(dimethylamino)-5- |
| | | methoxy-3,4-dihydro-2H-chromen-8-yl]- |
| | | 2-methylbenzenesulfonamide |
| 83 | 419 | N-[(3R)-3-(dimethylamino)-5-methoxy- |
| | | 3,4-dihydro-2H-chromen-8-yl]-2,3,5,6- |
| | | tetramethylbenzenesulfonamide |
| 84 | 381 | N-[(3R)-3-(dimethylamino)-5-methoxy- |
| | | 3,4-dihydro-2H-chromen-8-yl]-2- |
| | | fluorobenzenesulfonamide |
| 85 | 381 | N-[(3R)-3-(dimethylamino)-5-methoxy- |
| | | 3,4-dihydro-2H-chromen-8-yl]-4- |
| | | fluorobenzenesulfonamide |
| 86 | 431 | N-[(3R)-3-(dimethylamino)-5-methoxy- |
| | | 3,4-dihydro-2H-chromen-8-yl]-2- |
| | | (trifluoromethyl)benzenesulfonamide |
| 87 | 437 | 2,5-dichloro-N-[(3R)-3-(dimethylamino)- |
| | | 5-methoxy-3,4-dihydro-2H-chromen-8- |
| | | yl]thiophene-3-sulfonamide |
| 88 | 423 | N-[(3R)-3-(dimethylamino)-5-methoxy- |
| | | 3,4-dihydro-2H-chromen-8-yl]-3,4- |
| | | dimethoxybenzenesulfonamide |
| 89 | 391 | N-[(3R)-3-(dimethylamino)-5-methoxy- |
| | | 3,4-dihydro-2H-chromen-8-yl]-2,5- |
| | | dimethylbenzenesulfonamide |
| 90 | 393 | N-[(3R)-3-(dimethylamino)-5-methoxy- |
| | | 3,4-dihydro-2H-chromen-8-yl]-3- |
| | | methoxybenzenesulfonamide |
| 91 | 399 | N-[(3R)-3-(dimethylamino)-5-methoxy- |
| | | 3,4-dihydro-2H-chromen-8-yl]-2,5- |
| | | difluorobenzenesulfonamide |
| 92 | 531 | N-[(3R)-3-(dimethylamino)-5-methoxy- |
| | | 3,4-dihydro-2H-chromen-8-yl]-4-[2- |
| | | (phenylsulfonyl)ethyl]benzenesulfonamide |
| 93 | 447 | 8-chloro-N-[(3R)-3-(dimethylamino)-5- |
| | | methoxy-3,4-dihydro-2H-chromen-8- |
| | | yl]naphthalene-2-sulfonamide |
| 94 | 474 | N-[4-({[(3R)-3-(dimethylamino)-5- |
| | | methoxy-3,4-dihydro-2H-chromen-8- |
| | | yl]amino}sulfonyl)phenyl]-2,2,2- |
| | | trifluoroacetamide |
| 95 | 503 | N-[(3R)-3-(dimethylamino)-5-methoxy- |
| | | 3,4-dihydro-2H-chromen-8-yl]-2- |
| | | (phenylsulfonyl)benzenesulfonamide |
| 96 | 491 | 7-bromo-N-[(3R)-3-(dimethylamino)-5- |
| | | methoxy-3,4-dihydro-2H-chromen-8- |
| | | yl]naphthalene-1-sulfonamide |
| 97 | 480 | 4-(1,3-benzoxazol-2-yl)-N-[(3R)-3- |
| | | (dimethylamino)-5-methoxy-3,4-dihydro- |
| | | 2H-chromen-8-yl]benzenesulfonamide |
| 98 | 427 | N-[(3R)-3-(dimethylamino)-5-methoxy- |
| | | 3,4-dihydro-2H-chromen-8-yl]-4- |
| | | methylnaphthalene-1-sulfonamide |
| 99 | 447 | 5-chloro-N-[(3R)-3-(dimethylamino)-5- |
| | | methoxy-3,4-dihydro-2H-chromen-8- |
| | | yl]naphthalene-2-sulfonamide |
| 100 | 381 | N-[(3R)-3-(dimethylamino)-5-methoxy- |
| | | 3,4-dihydro-2H-chromen-8-yl]-1,2- |
| | | dimethyl-1H-imidazole-4-sulfonamide |
| 101 | 369 | N-[(3R)-3-(dimethylamino)-5-methoxy- |
| | | 3,4-dihydro-2H-chromen-8-yl]thiophene- |
| | | 3-sulfonamide |
| 102 | 433 | 2-chloro-N-[(3R)-3-(dimethylamino)-5- |
| | | methoxy-3,4-dihydro-2H-chromen-8-yl]- |
| | | 4,5-difluorobenzenesulfonamide |
| 103 | 441 | N-[(3R)-3-(dimethylamino)-5-methoxy- |
| | | 3,4-dihydro-2H-chromen-8-yl]-4- |
| | | (methylsulfonyl)benzenesulfonamide |
| 104 | 433 | 4-chloro-N-[(3R)-3-(dimethylamino)-5- |
| | | methoxy-3,4-dihydro-2H-chromen-8-yl]- |
| | | 2,5-difluorobenzenesulfonamide |
| 105 | 439 | N-[(3R)-3-(dimethylamino)-5-methoxy- |
| | | 3,4-dihydro-2H-chromen-8-yl]-1,1′- |
| | | biphenyl-4-sulfonamide |
| 106 | 422 | 2-chloro-4-cyano-N-[(3R)-3- |
| | | (dimethylamino)-5-methoxy-3,4-dihydro- |
| | | 2H-chromen-8-yl]benzenesulfonamide |
| 107 | 411 | 3-chloro-N-[(3R)-3-(dimethylamino)-5- |
| | | methoxy-3,4-dihydro-2H-chromen-8-yl]- |
| | | 4-methylbenzenesulfonamide |
| 108 | 455 | 4-bromo-N-[(3R)-3-(dimethylamino)-5- |
| | | methoxy-3,4-dihydro-2H-chromen-8-yl]- |
| | | 2-methylbenzenesulfonamide |
| 109 | 425 | 4-chloro-N-[(3R)-3-(dimethylamino)-5- |
| | | methoxy-3,4-dihydro-2H-chromen-8-yl]- |
| | | 2,5-dimethylbenzenesulfonamide |
| 110 | 417 | N-[(3R)-3-(dimethylamino)-5-methoxy- |
| | | 3,4-dihydro-2H-chromen-8-yl]-2,3,4- |
| | | trifluorobenzenesulfonamide |
| 111 | 419 | 4-butyl-N-[(3R)-3-(dimethylamino)-5- |
| | | methoxy-3,4-dihydro-2H-chromen-8- |
| | | yl]benzenesulfonamide |
| 112 | 411 | 1-(3-chlorophenyl)-N-[(3R)-3- |
| | | (dimethylamino)-5-methoxy-3,4-dihydro- |
| | | 2H-chromen-8-yl]methanesulfonamide |
| 113 | 417 | N-[(3R)-3-(dimethylamino)-5-methoxy- |
| | | 3,4-dihydro-2H-chromen-8-yl]-2,4,5- |
| | | trifluorobenzenesulfonamide |
| 114 | 439 | methyl 4-({[(3R)-3-(dimethylamino)-5- |
| | | methoxy-3,4-dihydro-2H-chromen-8- |
| | | yl]amino}sulfonyl)-2,5-dimethyl-3-furoate |
| 115 | 476 | 5-bromo-6-chloro-N-[(3R)-3- |
| | | (dimethylamino)-5-methoxy-3,4-dihydro- |
| | | 2H-chromen-8-yl]pyridine-3-sulfonamide |
| 116 | 429 | 3-chloro-N-[(3R)-3-(dimethylamino)-5- |
| | | methoxy-3,4-dihydro-2H-chromen-8-yl]- |
| | | 5-fluoro-2-methylbenzenesulfonamide |
| 117 | 469 | 4-bromo-N-[(3R)-3-(dimethylamino)-5- |
| | | methoxy-3,4-dihydro-2H-chromen-8-yl]- |
| | | 2-ethylbenzenesulfonamide |
| 118 | 456 | N-[(3R)-3-(dimethylamino)-5-methoxy- |
| | | 3,4-dihydro-2H-chromen-8-yl]-6- |
| | | phenoxypyridine-3-sulfonamide |
| 119 | 465 | 2,3,4-trichloro-N-[(3R)-3- |
| | | (dimethylamino)-5-methoxy-3,4-dihydro- |
| | | 2H-chromen-8-yl]benzenesulfonamide |
| 120 | 477 | 4-bromo-N-[(3R)-3-(dimethylamino)-5- |
| | | methoxy-3,4-dihydro-2H-chromen-8-yl]- |
| | | 2,5-difluorobenzenesulfonamide |
| 121 | 439 | N-[(3R)-3-(dimethylamino)-5-methoxy- |
| | | 3,4-dihydro-2H-chromen-8-yl]-1,1′- |
| | | biphenyl-3-sulfonamide |
| 122 | 439 | N-[(3R)-3-(dimethylamino)-5-methoxy- |
| | | 3,4-dihydro-2H-chromen-8-yl]-1,1′- |
| | | biphenyl-2-sulfonamide |
| 123 | 378 | N-[(3R)-3-(dimethylamino)-5-methoxy- |
| | | 3,4-dihydro-2H-chromen-8-yl]-1-pyridin- |
| | | 3-ylmethanesulfonamide |
| 124 | 467 | N-[(3R)-3-(dimethylamino)-5-methoxy- |
| | | 3,4-dihydro-2H-chromen-8-yl]-2,2- |
| | | diphenylethanesulfonamide |
| 125 | 403 | N-[(3R)-3-(dimethylamino)-5-methoxy- |
| | | 3,4-dihydro-2H-chromen-8-yl]-1- |
| | | benzofuran-2-sulfonamide |
| 126 | 476 | 4-chloro-N′-[(3R)-3-(dimethylamino)-5- |
| | | methoxy-3,4-dihydro-2H-chromen-8- |
| | | yl]benzene-1,3-disulfonamide |
| 127 | 433 | N-[(3R)-3-(dimethylamino)-5-methoxy- |
| | | 3,4-dihydro-2H-chromen-8-yl]-4- |
| | | pentylbenzenesulfonamide |
| 128 | 485 | N-[(3R)-3-(dimethylamino)-5-methoxy- |
| | | 3,4-dihydro-2H-chromen-8-yl]-3-(2- |
| | | methoxyphenoxy)benzenesulfonamide |
| 129 | 469 | N-[(3R)-3-(dimethylamino)-5-methoxy- |
| | | 3,4-dihydro-2H-chromen-8-yl]-4′- |
| | | methoxy-1,1′-biphenyl-3-sulfonamide |
| 130 | 327 | N-[(3R)-3-(dimethylamino)-5-methoxy- |
| | | 3,4-dihydro-2H-chromen-8- |
| | | yl]cyclopropanesulfonamide |
| 131 | 513 | 1-[3,5-bis(trifluoromethyl)phenyl]-N- |
| | | [(3R)-3-(dimethylamino)-5-methoxy-3,4- |
| | | dihydro-2H-chromen-8- |
| | | yl]methanesulfonamide |
| 132 | 431 | N-[(3R)-3-(dimethylamino)-5-methoxy- |
| | | 3,4-dihydro-2H-chromen-8-yl]-4- |
| | | fluoronaphthalene-1-sulfonamide |
| 133 | 399 | N-[(3R)-3-(dimethylamino)-5-methoxy- |
| | | 3,4-dihydro-2H-chromen-8-yl]-3,5- |
| | | difluorobenzenesulfonamide |
| 134 | 411 | N-[(3R)-3-(dimethylamino)-5-methoxy- |
| | | 3,4-dihydro-2H-chromen-8-yl]-3-fluoro-4- |
| | | methoxybenzenesulfonamide |
| 135 | 493 | N-[(3R)-3-(dimethylamino)-5-methoxy- |
| | | 3,4-dihydro-2H-chromen-8-yl]-5-[2- |
| | | (methylthio)pyrimidin-4-yl]thiophene-2- |
| | | sulfonamide |
| 136 | 531 | 1-[3-chloro-5-(trifluoromethyl)pyridin-2- |
| | | yl]-N-[(3R)-3-(dimethylamino)-5- |
| | | methoxy-3,4-dihydro-2H-chromen-8-yl]- |
| | | 1H-pyrrole-2-sulfonamide |
| 137 | 499 | 2,6-dichloro-N-[(3R)-3-(dimethylamino)- |
| | | 5-methoxy-3,4-dihydro-2H-chromen-8- |
| | | yl]-4- |
| | | (trifluoromethyl)benzenesulfonamide |
| 138 | 517 | N-[(3R)-3-(dimethylamino)-5-methoxy- |
| | | 3,4-dihydro-2H-chromen-8-yl]-5-[1- |
| | | methyl-3-(trifluoromethyl)-1H-pyrazol-5- |
| | | yl]thiophene-2-sulfonamide |
| 139 | 504 | N-[(3R)-3-(dimethylamino)-5-methoxy- |
| | | 3,4-dihydro-2H-chromen-8-yl]-5-[5- |
| | | (trifluoromethyl)isoxazol-3-yl]thiophene- |
| | | 2-sulfonamide |
| 140 | 449 | N-[(3R)-3-(dimethylamino)-5-methoxy- |
| | | 3,4-dihydro-2H-chromen-8-yl]-4-fluoro-2- |
| | | (trifluoromethyl)benzenesulfonamide |
| 141 | 449 | N-[(3R)-3-(dimethylamino)-5-methoxy- |
| | | 3,4-dihydro-2H-chromen-8-yl]-4-fluoro-3- |
| | | (trifluoromethyl)benzenesulfonamide |
| 142 | 417 | N-[(3R)-3-(dimethylamino)-5-methoxy- |
| | | 3,4-dihydro-2H-chromen-8-yl]-2,4,6- |
| | | trifluorobenzenesulfonamide |
| 143 | 436 | N-[(3R)-3-(dimethylamino)-5-methoxy- |
| | | 3,4-dihydro-2H-chromen-8-yl]-5-isoxazol- |
| | | 5-ylthiophene-2-sulfonamide |
| 144 | 422 | N-[(3R)-3-(dimethylamino)-5-methoxy- |
| | | 3,4-dihydro-2H-chromen-8-yl]-1-(3- |
| | | nitrophenyl)methanesulfonamide |
| 145 | 449 | N-[(3R)-3-(dimethylamino)-5-methoxy- |
| | | 3,4-dihydro-2H-chromen-8-yl]-2-fluoro-5- |
| | | (trifluoromethyl)benzenesulfonamide |
| 146 | 435 | N-[(3R)-3-(dimethylamino)-5-methoxy- |
| | | 3,4-dihydro-2H-chromen-8-yl]-5-methyl- |
| | | 2,1,3-benzothiadiazole-4-sulfonamide |
| 147 | 451 | N-[(3R)-3-(dimethylamino)-5-methoxy- |
| | | 3,4-dihydro-2H-chromen-8-yl]-5-fluoro-3- |
| | | methyl-1-benzothiophene-2-sulfonamide |
| 148 | 461 | 2,3-dichloro-N-[(3R)-3-(dimethylamino)- |
| | | 5-methoxy-3,4-dihydro-2H-chromen-8- |
| | | yl]-4-methoxybenzenesulfonamide |
| 149 | 411 | 1-(4-chlorophenyl)-N-[(3R)-3- |
| | | (dimethylamino)-5-methoxy-3,4-dihydro- |
| | | 2H-chromen-8-yl]methanesulfonamide |
| 150 | 431 | 2,3-dichloro-N-[(3R)-3-(dimethylamino)- |
| | | 5-methoxy-3,4-dihydro-2H-chromen-8- |
| | | yl]benzenesulfonamide |
| 151 | 403 | 5-chloro-N-[(3R)-3-(dimethylamino)-5- |
| | | methoxy-3,4-dihydro-2H-chromen-8- |
| | | yl]thiophene-2-sulfonamide |
| 152 | 411 | 2-chloro-N-[(3R)-3-(dimethylamino)-5- |
| | | methoxy-3,4-dihydro-2H-chromen-8-yl]- |
| | | 6-methylbenzenesulfonamide |
| 153 | 431 | 3,4-dichloro-N-[(3R)-3-(dimethylamino)- |
| | | 5-methoxy-3,4-dihydro-2H-chromen-8- |
| | | yl]benzenesulfonamide |
| 154 | 431 | 3,5-dichloro-N-[(3R)-3-(dimethylamino)- |
| | | 5-methoxy-3,4-dihydro-2H-chromen-8- |
| | | yl]benzenesulfonamide |
| 155 | 514 | 4-(3-chloro-2-cyanophenoxy)-N-[(3R)-3- |
| | | (dimethylamino)-5-methoxy-3,4-dihydro- |
| | | 2H-chromen-8-yl]benzenesulfonamide |
| 156 | 447 | 5-bromo-N-[(3R)-3-(dimethylamino)-5- |
| | | methoxy-3,4-dihydro-2H-chromen-8- |
| | | yl]thiophene-2-sulfonamide |
| 157 | 405 | N-[(3R)-3-(dimethylamino)-5-methoxy- |
| | | 3,4-dihydro-2H-chromen-8-yl]-4- |
| | | isopropylbenzenesulfonamide |
| 158 | 481 | 4-bromo-5-chloro-N-[(3R)-3- |
| | | (dimethylamino)-5-methoxy-3,4-dihydro- |
| | | 2H-chromen-8-yl]thiophene-2- |
| | | sulfonamide |
| 159 | 427 | 5-chloro-N-[(3R)-3-(dimethylamino)-5- |
| | | methoxy-3,4-dihydro-2H-chromen-8-yl]- |
| | | 2-methoxybenzenesulfonamide |
| 160 | 381 | N-[(3R)-3-(dimethylamino)-5-methoxy- |
| | | 3,4-dihydro-2H-chromen-8-yl]-3- |
| | | fluorobenzenesulfonamide |
| 161 | 454 | N-[2-chloro-4-({[(3R)-3-(dimethylamino)- |
| | | 5-methoxy-3,4-dihydro-2H-chromen-8- |
| | | yl]amino}sulfonyl)phenyl]acetamide |
| 162 | 445 | 2,4-dichloro-N-[(3R)-3-(dimethylamino)- |
| | | 5-methoxy-3,4-dihydro-2H-chromen-8- |
| | | yl]-5-methylbenzenesulfonamide |
| 163 | 432 | N-[(3R)-3-(dimethylamino)-5-methoxy- |
| | | 3,4-dihydro-2H-chromen-8-yl]-2-oxo- |
| | | 1,2,3,4-tetrahydroquinoline-6-sulfonamide |
| 164 | 445 | 2,4-dichloro-N-[(3R)-3-(dimethylamino)- |
| | | 5-methoxy-3,4-dihydro-2H-chromen-8- |
| | | yl]-6-methylbenzenesulfonamide |
| 165 | 399 | N-[(3R)-3-(dimethylamino)-5-methoxy- |
| | | 3,4-dihydro-2H-chromen-8-yl]-3,4- |
| | | difluorobenzenesulfonamide |
| 166 | 377 | N-[(3R)-3-(dimethylamino)-5-methoxy- |
| | | 3,4-dihydro-2H-chromen-8-yl]-2- |
| | | methylbenzenesulfonamide |
| 167 | 489 | N-[(3R)-3-(dimethylamino)-5-methoxy- |
| | | 3,4-dihydro-2H-chromen-8-yl]-4- |
| | | iodobenzenesulfonamide |

### Example 168

### (i) 3-Chloro-N-[(3R)-5-methoxy-3-pyrrolidin-1-yl-3,4-dihydro-2H-chromen-8-yl]-4-methylbenzenesulfonamide

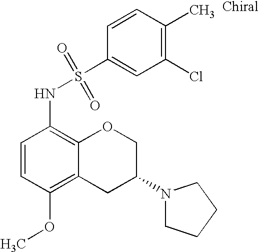

(3R)-5-Methoxy-3-pyrrolidin-1-ylchroman-8-amine (50 mg, 0.20 mmol) and 3-chloro-4-methylbenzensulfonyl chloride (40 mg, 0.18 mmol) were dissolved in dichloromethane (3 ml) and DIPEA (0.5 ml) was added. The mixture was stirred at ambient temperature over night. The solvent was evaporated and the residue was dissolved in methylene chloride. The organic phase was washed with saturated aqueous sodium hydrogen carbonate, dried (Na₂SO₄), filtered and the solvent was evaporated. The residue was purified by chromatography on silica using a gradient of CHCl₃/MeOH/NH₃ reaching from 0-10% of methanol containing ammonia (3%) to give a solid (40 mg, 51%). ¹H NMR (400 MHz, CDCl₃) δ ppm 7.70 (1H, d) 7.44 (1H, dd) 7.30 (1H, d) 7.22 (1H, d) 6.54 (1H, s) 6.39 (1H, d) 4.10-4.16 (1H, m) 3.80 (s, 3H) 3.39-3.45 (1H, m) 2.84-2.91 (1H, m) 2.56-2.69 (4H, m) 2.39 (3H, s) 2.28-2.36 (2H, m) 1.77-1.83 (4H, m); ESI-MS m/z M+H 437, 439.

### (ii) 1-[(3R)-8-Bromo-5-methoxy-3,4-dihydro-2H-chromen-3-yl]pyrrolidine

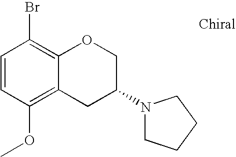

(3R)-8-Bromo-5-methoxychroman-3-amine (6.0 g, 20 mmol), 1,4-dibromobutane (4.9 ml, 41 mmol) and DIPEA (10 ml) were dissolved in DMF (50 ml). The mixture was heated at 60° C. for 10 hours. Aqueous sodium hydrogen carbonate was added and the mixture was extracted with EtOAc. The organic phase was washed with aqueous sodium hydrogen carbonate. The organic phase was extracted with hydrochloric acid (1 M). Aqueous sodium hydroxide (2 M) was added to the aqueous phase until basic pH was reached. The aqueous phase was extracted with EtOAc. The organic phase was dried (Na₂SO₄), filtered and the solvent evaporated. The residue was purified by chromatography on silica using a gradient of CHCl₃/MeOH/NH₃ reaching from 0-10% of methanol containing ammonia (3%) to give a solid (4.0 g, 65%). EI-MS m/z M+H 312, 314.

### (iii) (3R)—N-(Diphenylmethylene)-5-methoxy-3-pyrrolidin-1-ylchroman-8-amine

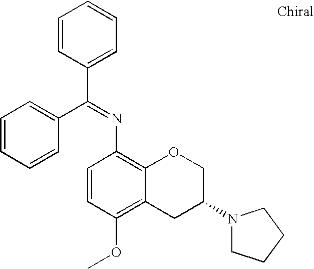

1-[(3R)-8-Bromo-5-methoxy-3,4-dihydro-2H-chromen-3-yl]pyrrolidine (1.3 g, 4.2 mmol), 1,1-diphenylmethanimine (0.76 g, 4.2 mmol), bis(2-diphenylphosphinophenyl)ether (0.11 g, 0.12 mmol) and sodium t-butoxide (1.3 g, 13 mmol) were mixed in toluene (20 ml) under argon atmosphere and the mixture was heated at 100° C. for 2 hours and then left at RT over night. Saturated aqueous sodium hydrogen carbonate was added and the mixture was extracted with EtOAc. The organic phase was washed with saturated aqueous sodium hydrogen carbonate (×3), dried (Na₂SO₄), filtered and the solvent was evaporated. The residue was purified by chromatography on silica using a gradient of CHCl₃/MeOH/NH₃ reaching from 0-10% of methanol containing ammonia (3%) to give an oil (1.4 g, 84%). API-MS m/z, M+H 413, 415.

### (iv) (3R)-5-Methoxy-3-pyrrolidin-1-ylchroman-8-amine

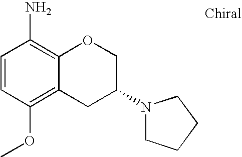

3R)—N-(Diphenylmethylene)-5-methoxy-3-pyrrolidin-1-ylchroman-8-amine (1.4 g, 3.4 mmol) was dissolved in THF (20 ml). Hydrochloric acid (1M, 6 ml) was added and the mixture was stirred at ambient temperature over night. Water (10 ml) and hydrochloric acid (1M, 3 ml) was added and the aqueous phase was washed with heptane and EtOAc. Aqueous sodium hydroxide (5M) was added to the aqueous phase until basic pH was reached. The aqueous phase was extracted with EtOAc (×3). The organic phase was dried (Na₂SO₄), filtered and the solvent was evaporated. The residue was purified by chromatography on silica using a gradient of CHCl₃/MeOH/NH₃ reaching from 0-10% of methanol containing ammonia (3%) to give a solid (0.6 g, 71%). ¹H NMR (400 MHz, CDCl₃) 8 ppm 6.54 (1H, d) 6.29 (1H, d) 4.43-4.49 (1H, m) 3.78-3.85 (1H, m) 3.76 (3H, s) 3.47 (2H, br. s.) 2.99-3.06 (1H, m) 2.49-2.79 (6H, m) 1.79-1.89 (4H, m); ESI-MS m/z M+H 249.

### Example 169

### 5-Chloro-N-[(3R)-5-methoxy-3-pyrrolidin-1-yl-3,4-dihydro-2H-chromen-8-yl]naphthalene-2-sulfonamide

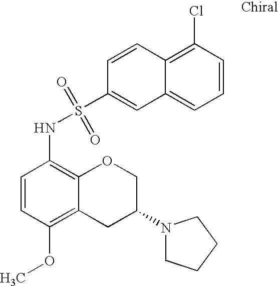

The title compound was prepared using the method in example 168 (i) to give a solid (40 mg, 50%). ¹H NMR (400 MHz, CD₃OD) 8 ppm 8.28 (1H, d) 8.11 (1H, d) 7.81-7.86 (2H, m) 7.73 (1H, d) 7.49-7.54 (1H, m) 7.23 (1H, d) 6.48 (1H, d) 3.76 (3H, s) 3.58-3.63 (1H, m) 3.00-3.06 (1H, m) 2.61-2.69 (1H, m) 2.32-2.38 (4H, m) 2.10-2.19 (1H, m) 1.64-1.72 (5H, m); ESI-MS m/z M+H 473,475.

### Example 170

### (i) (2S)-5-{[(3-Bromophenyl)sulfonyl]amino}-N,N-dimethyl-1,2,3,4-tetrahydronaphthalen-2-ammonium Acetate

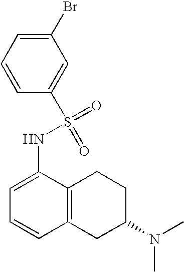

A 10 M solution of KOH (0.25 ml, 2.5 mmol) was added to a suspension of the crude 3-bromo-N-[(3-bromophenyl)sulfonyl]-N-[(6S)-6-(dimethylamino)-5,6,7,8-tetrahydronaphthalen-1-yl]benzenesulfonamide (0.094 mmol) in MeOH/H₂O (1 ml: 1 ml) and the reaction mixture was heated at 50° C. for two hours. The solvent was evaporated, aqueous saturated NaHCO₃ solution was added and the mixture was extracted with EtOAc (×4). The organic layers were combined and evaporated. The product was purified by preparative HPLC afford the title compound was obtained as a solid (21 mg, 54%).

¹H NMR (400 MHz, CD₃OD) δ ppm 7.71-7.82 (m, 2H), 7.61-7.69 (m, 1H), 7.43 (t, 1H), 6.99-7.13 (m, 2H), 6.76-6.86 (m, 1H), 3.10-3.30 (m, 2H), 2.83-3.03 (m, 2H), 2.75 (s, 6H), 2.49-2.66 (m, 1H), 2.13-2.26 (m, 1H), 1.87-1.97 (s, 3H), 1.53-1.67 (m, 1H). MS m/z M+H 409, 411, M-1 407, 409.

### (ii) N-[(6S)-6-Amino-5,6,7,8-tetrahydronaphthalen-1-yl]-2-hydroxy-2-methylpropanamide

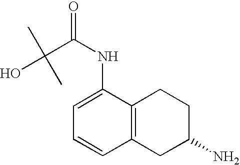

A two-neck round-bottom flask equipped with a condenser was charged with N-[(6S)-6-(dibenzylamino)-5,6,7,8-tetrahydronaphthalen-1-yl]-2-hydroxy-2-methylpropanamide (747 mg, 1.7 mmol) and ammonium formate (3.8 g, 60 mmol). MeOH (25 ml) was added, the flask was flushed with N₂ and 10% Pd on carbon (75 mg) was added. The reaction mixture was heated at 50° C. under vigorous stirring overnight. The reaction mixture was cooled down, the solid was filtered off on Celite and solvent was evaporated under reduced pressure. The resulting solid was dissolved in EtOAc and washed with 1M aqueous Na₂CO₃. The solvent was evaporated under reduced pressure to afford the title compound that was directly used in the next step. MS m/z M+H 249, M−H 247.

### (iii) N-[(6S)-6-(Dimethylamino)-5,6,7,8-tetrahydronaphthalen-1-yl]-2-hydroxy-2-methylpropanamide

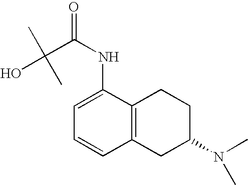

Sodium cyanoborohydride (0.53 g, 8.5 mmol) was added to a solution of the crude N-[(6S)-6-amino-5,6,7,8-tetrahydronaphthalen-1-yl]-2-hydroxy-2-methylpropanamide (0.42 g, 1.7 mmol) and formaldehyde (33% in water, 1.1 ml, 14 mmol) in MeOH (5 ml) at 0° C. AcOH (60 μL) was added and the reaction stirred at 0° C. for two hours. The ice bath was removed and the reaction mixture was stirred overnight. The solvent was evaporated under reduced pressure, 1M aqueous solution of Na₂CO₃ was added and the aqueouse phase was extracted with EtOAc (×4). Brine was added to the aqueous phase which was extracted with additional EtOAc (×2). The organic phases were combined and dried over Na₂SO₄ and the solvent was removed under reduced pressure. The residue was purified by chromatography on silica using a gradient of CHCl₃/MeOH/NH₃ reaching from 0-10% of methanol containing ammonia (3%) to afford the title compound (370 mg, 78%). ¹H NMR (400 MHz, CD₃OD) δ ppm 7.45 (d, 1H), 7.12 (t, 1H), 6.98 (d, 1H), 2.94-3.06 (m, 1H), 2.71-2.91 (m, 2H), 2.51-2.70 (m, 2H), 2.37 (s, 6H), 2.13-2.27 (m, 1H), 1.54-1.71 (m, 1H), 1.47 (s, 6H). MS m/z M+H 277, M−H 275.

### (iv) (6S)—N⁶,N⁶-Dimethyl-5,6,7,8-tetrahydronaphthalene-1,6-diammonium Hydrochloride

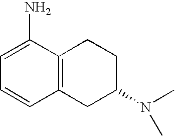

Concentrated hydrochloric acid (1.2 ml) was added to a solution of N-[(6S)-6-(dimethylamino)-5,6,7,8-tetrahydronaphthalen-1-yl]-2-hydroxy-2-methylpropanamide (26 mg, 0.094 mmol) in EtOH/water (1 ml:0.8 ml). The reaction mixture was refluxed overnight and the solvents evaporated under reduced pressure. The solid was taken up in acetonitrile and stripped to afford the title compound that was used in the next step without further purification.

### (v) 3-Bromo-N-[(3-bromophenyl)sulfonyl]-N-[(6S)-6-(dimethylamino)-5,6,7,8-tetrahydronaphthalen-1-yl]benzenesulfonamide

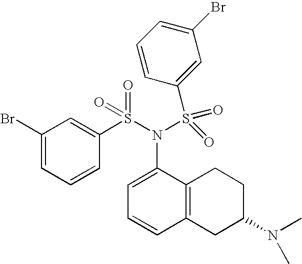

3-Bromobenzenesulfonyl chloride (0.2 mmol, 34 μL) was added to a suspension of crude (6S)—N⁶,N⁶-dimethyl-5,6,7,8-tetrahydronaphthalene-1,6-diammonium hydrochloride (0.094 mmol) and triethylamine (0.4 mmol, 58 μL) in acetonitrile/DMF (1 ml:0.15 ml). The mixture was stirred at ambient temperature overnight. The solvent was evaporated under reduced pressure to afford the crude 3-bromo-N-[(3-bromophenyl)sulfonyl]-N-[(6S)-6-(dimethylamino)-5,6,7,8-tetrahydronaphthalen-1-yl]benzenesulfonamide that was used directly in the next step.

MS m/z M+H 629.

### Example 171

### (i) N-[(6S)-4-Bromo-6-(dimethylamino)-5,6,7,8-tetrahydronaphthalen-1-yl]benzenesulfonamide

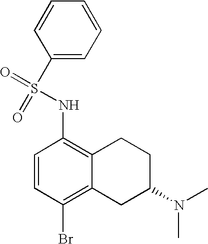

A 10 M aqueous solution of KOH (10 ml) was added to a solution of crude N-[(6S)-4-bromo-6-(dimethylamino)-5,6,7,8-tetrahydronaphthalen-1-yl]-N(phenylsulfonyl)benzenesulfonamide (0.09 mmol) in MeOH (15 ml). The reaction was stirred for three hours at 50° C., cooled down to room temperature and neutralized with concentrated hydrochloride acid. A 1M NaHCO₃ solution was added and the aqueous phase was extracted with EtOAc (×3). The organic phases were combined and the solvent was evaporated under reduced pressure. The product was purified by preparative HPLC to afford the title compound as a solid (51 mg, 25%). ¹H NMR (400 MHz, CD₃CN) δ ppm 7.66 (d, 2H), 7.53-7.61 (m, 1H), 7.47 (t, 2H), 7.28 (d, 1H), 6.83 (d, 1H), 2.78-2.95 (m, 2H), 2.38-2.67 (m, 3H), 2.28-2.37 (m, 1H), 2.26 (s, 6H), 1.82-1.91 (m, 1H), 1.22-1.30 (m, 1H). MS m/z M+H 409, 411 M−H 407, 409.

### (ii) N-[(6S)-4-Bromo-6-(dimethylamino)-5,6,7,8-tetrahydronaphthalen-1-yl]-2-hydroxy-2-methylpropanamide

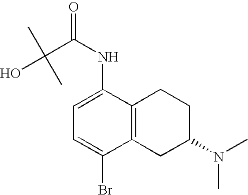

A solution of Br₂ (1.1 mmol, 57 μL) in AcOH (5 ml) was added dropwise to a solution of N-[(6S)-6-(dimethylamino)-5,6,7,8-tetrahydronaphthalen-1-yl]-2-hydroxy-2-methylpropanamide (300 mg, 1.08 mmol) in AcOH (10 ml). The reaction was stirred for two hours, additional Br₂ (0.1 mmol) was added and the reaction stirred for four more hours. The reaction was quenched with sodium thiosulfate and the solvent was evaporated. Water was added and the aqueous solution was extracted twice dichloromethane (×2). The organic phases were combined, dried over Na₂SO₄ and the solvent was evaporated to afford the crude product. MS m/z M+H 355, 357, M−H 353, 355.

### (iii) (6S)-4-Bromo-N⁶,N⁶-dimethyl-5,6,7,8-tetrahydronaphthalene-1,6-diammonium dichloride

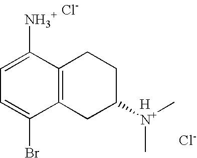

N-[(6S)-4-Bromo-6-(dimethylamino)-5,6,7,8-tetrahydronaphthalen-1-yl]-2-hydroxy-2-methylpropanamide was refluxed for four hours in HCl (8 ml, 10 M in H₂O), water (8 ml) and MeOH (5 ml). The solvents were evaporated under reduced pressure to afford the title compound.

¹H NMR (400 MHz, DMSO-d₆) δ ppm 11.37 (br. s., 1H), 9.90 (br. s., 3H), 7.27 (d, 1H), 6.66 (d, 1H), 4.50-4.59 (m, 1H), 4.29-4.40 (m, 1H), 3.01-3.13 (m, 1H), 2.86-2.97 (m, 1H), 2.77 (s, 6H). MS m/z M+H 269, 271.

### (iv) N-[(6S)-4-Bromo-6-(dimethylamino)-5,6,7,8-tetrahydronaphthalen-1-yl]-N(phenylsulfonyl)benzenesulfonamide

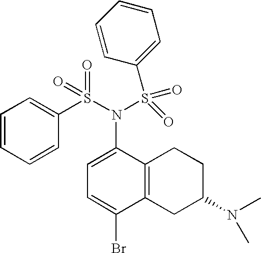

Benzenesulfonyl chloride (1.5 mmol, 189 μL was added in two portion to a solution of (6S)-4-bromo-N⁶,N⁶-dimethyl-5,6,7,8-tetrahydronaphthalene-1,6-diammonium dichloride (0.5 mmol) and triethylamine (5 mmol, 721 μL) in acetonitrile/dichloromethane (4 ml:2 ml) at ambient temperature. The reaction was stirred overnight and the solvents were evaporated under reduced pressure to afford the title compound that was used without purification. MS m/z M+H 549, 551.

### Example 172

### (i) N-[(6S)-4-Bromo-6-(dimethylamino)-5,6,7,8-tetrahydronaphthalen-1-yl]-3-chloro-4-fluorobenzenesulfonamide

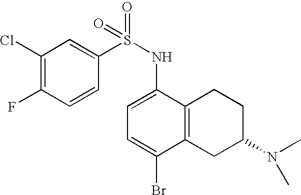

The title compound was synthesized using the same procedure as example 171 (i). The title compound was isolated in 115 mg (50%) yield.

¹H NMR (600 MHz, DMSO-d₆) δ ppm 7.61-7.68 (m, 1H), 7.54 (t, 1H), 7.24 (d, 1H), 6.72 (d, 1H), 2.72-2.84 (m, 2H), 2.51-2.65 (m, 2H), 2.35-2.46 (m, 1H), 2.30 (s, 6H), 1.85-1.90 (m, 1H), 1.29-1.42 (m, 1H).

MS m/z M+H 463, M−H 463.

### (ii) N-[(6S)-4-Bromo-6-(dimethylamino)-5,6,7,8-tetrahydronaphthalen-1-yl]-3-chloro-N-[(3-chloro-4-fluorophenyl)sulfonyl]-4-fluorobenzenesulfonamide

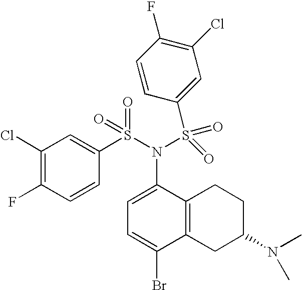

3-Chloro-4-fluorobenzenesulfonyl chloride (2 mmol, 286 μL) was added in two portions to a solution of (6S)-4-bromo-N⁶,N⁶-dimethyl-5,6,7,8-tetrahydronaphthalene-1,6-diammonium dichloride and triethylamine (5 mmol, 721 μL) in acetonitrile/dichloromethane (4 ml:2 ml) at ambient temperature. The reaction mixture was stirred overnight and the solvents were evaporated under reduced pressure to afford the crude title compound that was used without purification. MS m/z M+H 655, M−H 653.

### Example 173

### (i) 4-bromo-N-[(6S)-6-(dimethylamino)-4-methoxy-5,6,7,8-tetrahydronaphthalen-1-yl]-2-ethylbenzenesulfonamide

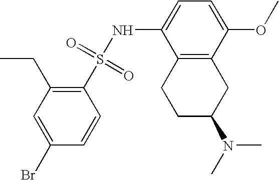

To a solution of 4-bromo-2-ethylbenzenesulfonyl chloride (28 mg, 0.10 mmol) in 1-methyl-2-pyrrolidinone (200 μL) was added a solution of (6S)-4-methoxy-N⁶,N⁶-dimethyl-5,6,7,8-tetrahydronaphthalene-1,6-diamine (20 mg, 0.10 mmol) in 1-methyl-2-pyrrolidizoize (200 μL) and triethylamine (42 μL, 0.30 mmol). The reaction mixture was shaken for 18 hours at ambient temperature and the volatiles were removed under vacuum. The crude product was purified first using polymer supported tosic(65) resin, loading as a solution in methanol (500 μL) followed by washing with excess methanol (2.0 ml) and finally eluting with 1M ammonia solution in methanol (1.0 ml). The methanol was removed under vacuum and the residue was further purified using preparative HPLC to give the named product (16.5 mg).

¹H NMR (500 MHz, DMSO-d₆) δ 7.68 (s, 1H), 7.53 (d, 1H), 6.65 (d, 1H), 6.58 (d, 1H), 3.71 (s, 3H), 2.93 (q, 2H), 2.82-2.64 (m, 3H), 2.39-2.30 (m, 2H), 2.20 (s, 6H), 1.87-1.82 (m, 1H), 1.27-1.22 (m, 1H), 1.18 (t, 3H).

MS m/z (APCI+) M+H 467 and 469

### (ii) [(2S)-8-Methoxy-1,2,3,4-tetrahydro-naphthalen-2-yl]-dimethyl-amine

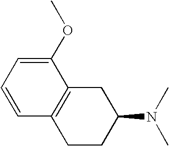

To a solution of [(2S)-8-methoxy-1,2,3,4-tetrahydro-naphthalen-2-yl]-amine (8.0 g, 37.0 mmol) in methanol (100 ml) was added aqueous formaldehyde (37%, 22 ml, 300 mmol) and acetic acid (10 ml). To this mixture was added sodium cyanoborohydride (19.0 g, 200 mmol) in portions keeping the temperature below 40° C. It was stirred overnight at ambient temperature and the solvent was removed under vacuum. The residue was partitioned between ethyl acetate (50 ml) and aqueous sodium hydroxide (2 M, 50 ml) followed by extracting the aqueous layer with ethyl acetate (3×30 ml). The combined organic layers were washed with saturated sodium chloride solution, dried over sodium sulphate, filtered and solvent removed under vacuum. The excess formaldehyde was removed by the use of SCX resin (loading as a solution in methanol and the resin was thoroughly washed with methanol, the product was eluted with 1M ammonia in methanol and evaporated under vacuum to dryness). The crude material was purified by column chromatography (silica, 2.5% methanol in dichloromethane) to give the named compound as an oil (7.30 g, 96%). ¹H NMR (400 MHz, CDCl₃) δ 7.08 (t, 1H), 6.71 (d, 1H), 6.65 (d, 1H), 3.81 (s, 3H), 3.03-2.97 (m, 1H), 2.88-2.80 (m, 2H), 2.59-2.52 (m, 1H), 2.48-2.41 (m, 1H), 2.38 (s, 6H), 2.10-2.05 (m, 1H), 1.57 (ddd, 1H).

### (iii) (2S)-5-Bromo-8-methoxy-N,N-dimethyl-1,2,3,4-tetrahydronaphthalen-2-amine

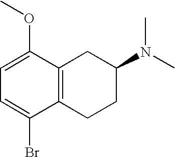

To a solution of [(2S)-8-methoxy-1,2,3,4-tetrahydro-naphthalen-2-yl]-dimethyl-amine (5.61 g, 23.21 mmol) in acetic acid (145 ml) was added sodium acetate (5.71 g, 69.63 mmol) and it was stirred at RT until most of the sodium acetate dissolved. A solution of bromine (3.90 g, 1.26 ml, 24.37 mmol) in acetic acid was added dropwise over a period of 6 hours. The white precipitate formed was filtered off and washed with water followed by Et₂O. It was dried under vacuum to give the HBr salt of the title compound (8.14 g) as a solid. ¹H NMR (300 MHz, CDCl₃) δ 7.33 (d, 1 μl), 6.57 (d, 1H), 3.80 (s, 3H), 3.03 (dd, 1H), 3.00-2.95 (m, 1H), 2.70-2.58 (m, 1H), 2.54-2.42 (m, 2H), 2.37 (s, 6H), 2.15-2.07 (m, 1H), 1.65-1.51 (m, 1H).

### (iv) (6S)-4-methoxy-N⁶,N⁶-dimethyl-5,6,7,8-tetrahydronaphthalene-1,6-diamine, Method A

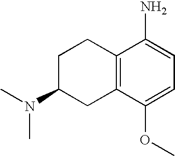

To a solution of [(2S)-5-bromo-8-methoxy-1,2,3,4-tetrahydronaphthalen-2-yl]dimethylamine (6.25 g, 22.0 mmol) in dimethylformamide (31.0 ml) in an autoclave container was added a concentrated aqueous ammonia solution (31.0 ml) and copper powder (1.68 g, 26.4 mmol). The container was then sealed and the reaction was heated to 110° C. for 18 hours with stirring. After it has cooled to RT, the reaction mixture was poured into saturated ammonium chloride solution (70 ml) and the aqueous layer was extracted with dichloromethane (3×70 ml). The combined organic layers were washed with a saturated ammonium chloride solution (100 ml) followed by a saturated sodium chloride solution (100 ml) and was dried over sodium sulphate, filtered and concentrated in vacuo to give an oil (4.78 g). The presence of the title compound was confirmed by LC/MS (purity >95%) and the crude material was used immediately in the next step.

¹H NMR (400 MHz, CDCl₃) δ 6.58 (d, 1H), 6.52 (d, 1H), 2.72-2.67 (m, 1H), 3.76 (s, 3H), 3.04-2.99 (d, 1H), 2.67-2.41 (m, 2H), 2.39 (s, 6H), 2.18-2.13 (m, 1H), 1.63-1.59 (m, 1H). MS m/z (APCI+) M+H 221

### (iii) (2S)-8-Methoxy-N,N-dimethyl-5-nitro-1,2,3,4-tetrahydronaphthalen-2-amine

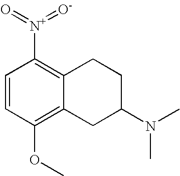

To a cooled (0° C.) solution of [(2S)-8-methoxy-1,2,3,4-tetrahydro-naphthalen-2-yl]-dimethyl-amine (0.495 g, 2.40 mmol) in trifluoroacetic acid (14.5 ml) was added sodium nitrate (0.205 g, 2.40 mmol) in portions and it was stirred at RT for 1 hr. It was worked up by neutralising with aqueous ammonia solution until pH=10 and the aqueous solution was extracted with dichloromethane (3×100 ml). The combined organic layers were washed with saturated sodium chloride solution, dried over sodium sulphate, filtered and the solvent was removed under vacuum. The crude brown oil was first trituated with diethyl ether and the solid residue was discarded. After removing the solvent under vacuum, the crude product was purified by column chromatography (silica, 5% methanol in dichloromethane) to give the title compound (412 mg, 68.6% yield) as an oil. ¹H NMR (400 MHz, CDCl₃) δ 7.94 (d, J=9.0 Hz, 1H), 6.74 (d, J=9.0 Hz, 1H), 3.91 (s, 3H), 3.26-3.19 (m, 1H), 3.11-3.07 (m, 1H), 3.05-2.94 (m, 2H), 2.56-2.51 (m, 1H), 2.40 (s, 6H), 2.19-2.12 (m, 1H), 1.58-1.48 (m, 1H). m/z (APCI+) 251 (M+H)⁺

### (iv) (6S)-4-methoxy-N⁶,N⁶-dimethyl-5,6,7,8-tetrahydronaphthalene-1,6-diamine, Method B

To a solution of (2S)-8-methoxy-N,N-dimethyl-5-nitro-1,2,3,4-tetrahydronaphthalen-2-amine (0.100 g, 0.40 mmol) in ethanol (2.5 ml) was added palladium on carbon (10%, 12 mg). The reaction was stirred under an atmosphere of hydrogen (4 bars) for 18 hours. It was worked up by filtering through a Celite® pad and it was washed thoroughly with excess ethanol. The solvent of the filtrate was removed under vacuum to give the crude product as an oil (79 mg). No further purification was done. ¹H NMR (400 MHz, CDCl₃) d 6.58 (d, 1H), 6.52 (d, 1H), 2.72-2.67 (m, 1H), 3.76 (s, 3H), 3.04-2.99 (d, 1H), 2.67-2.41 (m, 2H), 2.39 (s, 6H), 2.18-2.13 (m, 1H), 1.63-1.59 (m, 1H). m/z (APCI+) 221 (M+H)⁺

### Example 174 to 194

The following compounds were synthesized in an analogous method to example 173 (i)

| | | | NMR Data ¹H NMR |
|---|---|---|---|
| | | MS | (500.075 MHz, |
| Example | Name | M + H | DMSO-d₆) |
| 174 | 5-bromo-6-chloro-N-[(6S)-6- | 474 | 8.54 (s, 1H), 8.21 (s, |
| | (dimethylamino)-4-methoxy-5,6,7,8- | | 1H), 6.67 (s, 1H), |
| | tetrahydronaphthalen-1-yl]pyridine- | | 6.53 (s, 1H), 3.72 (s, |
| | 3-sulfonamide | | 3H), 2.88-2.64 (m, |
| | | | 3H), 2.39-2.30 (m, |
| | | | 2H), 2.27 (s, 6H), |
| | | | 1.92-1.85 (m, 1H), |
| | | | 1.36-1.23 (m, 1H). |
| 175 | N-[(6S)-6-(dimethylamino)-4- | 418 | 7.09-7.06 (m, 1H), |
| | methoxy-5,6,7,8- | | 7.04-7.03 (m, 2H), |
| | tetrahydronaphthalen-1-yl]-2,3- | | 7.00 (d, 1H), 6.68 (s, |
| | dihydro-1,4-benzodioxine-6- | | 1H), 4.32-4.26 (m, |
| | sulfonamide | | 4H), 3.73 (s, 3H), |
| | | | 2.86-2.66 (m, 3H), |
| | | | 2.34-2.24 (m, 2H), |
| | | | 2.20 (s, 6H), |
| | | | 1.85-1.80 (m, 1H), |
| | | | 1.25-1.18 (m, 1H). |
| 176 | N-[(6S)-6-(dimethylamino)-4- | 437 | 7.91 (d, 1H), 7.65 (t, |
| | methoxy-5,6,7,8- | | 1H), 7.57 (t, 1H), |
| | tetrahydronaphthalen-1-yl]-1,1′- | | 7.33-7.26 (m, 3H), |
| | biphenyl-2-sulfonamide | | 7.10 (d, 1H), |
| | | | 6.61 (dd, 2H), 6.54 (dd, |
| | | | 2H), 3.72 (s, 3H), |
| | | | 2.79 (m, 3H), |
| | | | 2.41-2.30 (m, 2H), 2.22 (s, |
| | | | 6H), 1.85-1.80 (m, |
| | | | 1H), 1.26-1.21 (m, |
| | | | 1H) |
| 177 | N-[(6S)-6-(dimethylamino)-4- | 376 | 8.54 (s, 1H), 7.79 (d, |
| | methoxy-5,6,7,8- | | 1H), 7.41 (t, 1H), |
| | tetrahydronaphthalen-1-yl]-1-pyridin- | | 7.08 (d, 1H), 6.77 (d, |
| | 3-ylmethanesulfonamide | | 1H), 6.54 (s, 1H), |
| | | | 4.46 (s, 2H), 3.78 (s, |
| | | | 3H), 2.84-2.64 (m, |
| | | | 3H), 2.45-2.33 (m, |
| | | | 2H), 2.24 (s, 6H), |
| | | | 1.99-1.93 (m, 1H), |
| | | | 1.43-1.35 (m, 1H) |
| 178 | 4-chloro-N¹-[(6S)-6- | 474 | 8.26 (s, 1H), 7.84 (d, |
| | (dimethylamino)-4-methoxy-5,6,7,8- | | 1H), 7.75 (d, 1H), |
| | tetrahydronaphthalen-1-yl]benzene- | | 6.68 (d, 1H), 6.60 (d, |
| | 1,3-disulfonamide | | 1H), 3.72 (s, 3H), |
| | | | 2.80-2.64 (m, 3H), |
| | | | 2.43-2.30 (m, 2H), |
| | | | 2.21 (s, 6H), |
| | | | 1.87-1.82 (m, 1H), |
| | | | 1.29-1.21 (m, 1H) |
| 179 | 5-chloro-N-[(6S)-6-(dimethylamino)- | 445 | 8.59 (d, 1H), 8.51 (d, |
| | 4-methoxy-5,6,7,8- | | 1H), 8.10 (d, 1H), |
| | tetrahydronaphthalen-1- | | 7.86 (d, 1H), 7.76 (t, |
| | yl]naphthalene-1-sulfonamide | | 1H), 7.60 (t, 1H), |
| | | | 6.58 (d, 1H), 6.47 (d, |
| | | | 1H), 3.68 (s, 3H), |
| | | | 2.81-2.73 (m, 1H), |
| | | | 2.59-2.22 (m, 4H), |
| | | | 2.14 (s, 6H), |
| | | | 1.68-1.61 (m, 1H), |
| | | | 1.06-0.97 (m, 1H) |
| 180 | N-[(6S)-6-(dimethylamino)-4- | 447 | 7.98-7.95 (m, 1H), |
| | methoxy-5,6,7,8- | | 7.85-7.82 (m, 1H), |
| | tetrahydronaphthalen-1-yl]-4-fluoro- | | 7.76-7.70 (m, 1H), |
| | 3- | | 6.68 (dd, 1H), |
| | (trifluoromethyl)benzenesulfonamide | | 6.63 (dd, 1H), 3.72 (s, |
| | | | 3H), 2.75-2.66 (m, |
| | | | 1H), 2.59-2.29 (m, |
| | | | 4H), 2.19 (s, 6H), |
| | | | 1.84-1.78 (m, 1H), |
| | | | 1.25-1.18 (m, 1H) |
| 181 | N-[(6S)-6-(dimethylamino)-4- | 449 | 8.10-8.06 (m, 1H), |
| | methoxy-5,6,7,8- | | 7.73 (d, 1H), 7.43 (t, |
| | tetrahydronaphthalen-1-yl]-5-fluoro- | | 1H), 6.81 (d, 1H), |
| | 3-methyl-1-benzothiophene-2- | | 6.69 (d, 1H), 3.73 (s, |
| | sulfonamide | | 3H), 2.81-2.63 (m, |
| | | | 3H), 2.59-2.49 (m, |
| | | | 1H), 2.39-2.29 (m, |
| | | | 1H), 2.22 (s, 16H), |
| | | | 2.11 (s, 6H), |
| | | | 1.69-1.62 (m, 1H), |
| | | | 1.13-1.04 (m, 3H) |
| 182 | 1-(4-chlorophenyl)-N-[(6S)-6- | 409 | 7.44 (d, 1H), 7.38 (d, |
| | (dimethylamino)-4-methoxy-5,6,7,8- | | 1H), 7.07 (d, 1H), |
| | tetrahydronaphthalen-1- | | 6.76 (d, 1H), 4.40 (s, |
| | yl]methanesulfonamide | | 2H), 3.78 (s, 3H), |
| | | | 2.84-2.64 (m, 3H), |
| | | | 2.58-2.48 (m, 1H), |
| | | | 2.40-2.32 (m, 1H), |
| | | | 2.24 (s, 6H), |
| | | | 1.99-1.93 (m, 1H), |
| | | | 1.43-1.34 (m, 1H). |
| 183 | 2-chloro-4-cyano-N-[(6S)-6- | 420 | 8.31-8.26 (m, 1H), |
| | (dimethylamino)-4-methoxy-5,6,7,8- | | 7.95 (d, 1H), 7.92 (d, |
| | tetrahydronaphthalen-1- | | 1H), 6.60-6.55 (m, |
| | yl]benzenesulfonamide | | 2H), 3.69 (s, 3H), |
| | | | 2.80-2.62 (m, 3H), |
| | | | 2.52-2.32 (m, 2H), |
| | | | 2.26 (s, 6H), |
| | | | 1.96-1.90 (m, 1H), |
| | | | 1.37-1.29 (m, 1H). |
| 184 | 6-chloro-N-[(6S)-6-(dimethylamino)- | 441 | 7.58-7.55 (m, 1H), |
| | 4-methoxy-5,6,7,8- | | 7.50-7.48 (m, 1H), |
| | tetrahydronaphthalen-1- | | 6.71 (d, 1H), 6.65 (d, |
| | yl]imidazo[2,1-b][1,3]thiazole-5- | | 1H), 3.71 (s, 3H), |
| | sulfonamide | | 2.78-2.61 (m, 3H), |
| | | | 2.58-2.37 (m, 2H), |
| | | | 2.26 (s, 6H), |
| | | | 1.88-1.81 (m, 1H), |
| | | | 1.26-1.19 (m, 1H). |
| 185 | N-[(6S)-6-(dimethylamino)-4- | 439 | 8.28 (d, 1H), |
| | methoxy-5,6,7,8- | | 7.97-7.93 (m, 2H), |
| | tetrahydronaphthalen-1-yl]-2- | | 7.86-7.81 (m, 1H), 6.65 (d, |
| | (methylsulfonyl)benzenesulfonamide | | 1H), 6.62 (d, 1H), |
| | | | 3.71 (s, 3H), |
| | | | 2.80-2.62 (m, 3H), |
| | | | 2.58-2.49 (m, 1H), 2.41 (s, |
| | | | 3H), 2.37-2.26 (m, |
| | | | 1H), 2.20 (s, 6H), |
| | | | 1.86-1.81 (m, 1H), |
| | | | 1.27-1.20 (m, 1H). |
| 186 | 7-chloro-N-[(6S)-6-(dimethylamino)- | 437 | 7.83 (d, 1H), 7.79 (d, |
| | 4-methoxy-5,6,7,8- | | 1H), 6.65 (d, 1H), |
| | tetrahydronaphthalen-1-yl]-2,1,3- | | 6.59 (d, 1H), 3.70 (s, |
| | benzoxadiazole-4-sulfonamide | | 3H), 2.79-2.63 (m, |
| | | | 1H), 2.60-2.48 (m, |
| | | | 1H), 2.39-2.31 (m, |
| | | | 1H), 2.26 (s, 6H), |
| | | | 1.88-1.81 (m, 1H), |
| | | | 1.28-1.22 (m, 1H). |
| 187 | 4,5-dibromo-N-[(6S)-6- | 523 | 7.26 (s, 1H), 6.81 (d, |
| | (dimethylamino)-4-methoxy-5,6,7,8- | | 1H), 6.69 (d, 1H), |
| | tetrahydronaphthalen-1-yl]thiophene- | | 3.73 (s, 3H), |
| | 2-sulfonamide | | 2.72-2.56 (m, 3H), |
| | | | 2.41-2.30 (m, 2H), 2.29 (s, |
| | | | 6H), 1.93-1.87 (m, |
| | | | 1H), 1.36-1.28 (m, |
| | | | 1H). |
| 188 | 5-bromo-N-[(6S)-6-(dimethylamino)- | 469 | 7.79 (dd, 1H), |
| | 4-methoxy-5,6,7,8- | | 7.58 (d, 1H), 7.25 (d, 1H), |
| | tetrahydronaphthalen-1-yl]-2- | | 6.65-6.61 (m, 2H), |
| | methoxybenzenesulfonamide | | 3.91 (s, 3H), 3.71 (s, |
| | | | 3H), 2.80-2.74 (m, |
| | | | 1H), 2.73-2.62 (m, |
| | | | 2H), 2.59-2.50 (m, |
| | | | 1H), 2.40-2.29 (m, |
| | | | 1H), 2.21 (s, 6H), |
| | | | 1.93-1.87 (m, 1H), |
| | | | 1.34-1.25 (m, 1H). |
| 189 | N-[(6S)-6-(dimethylamino)-4- | 453 | 7.59 (d, 3H), 7.46 (t, |
| | methoxy-5,6,7,8- | | 2H), 7.25 (t, 1H), |
| | tetrahydronaphthalen-1-yl]-4- | | 7.11-7.07 (m, 4H), |
| | phenoxybenzenesulfonamide | | 6.72-6.68 (m, 2H), |
| | | | 3.73 (s, 3H), |
| | | | 2.74-2.63 (m, 3H), |
| | | | 2.58-2.22 (m, 2H), |
| | | | 2.20 (6H), 1.86-1.80 (m, |
| | | | 1H), 1.25-1.17 (m, |
| | | | 1H). |
| 190 | 1-acetyl-5-bromo-N-[(6S)-6- | 522 | 8.55 (s, 1H), 7.72 (s, |
| | (dimethylamino)-4-methoxy-5,6,7,8- | | 1H), 6.63-6.58 (m, |
| | tetrahydronaphthalen-1-yl]indoline- | | 2H), 4.17-4.11 (m, |
| | 6-sulfonamide | | 2H), 3.70 (s, 3H), |
| | | | 3.25-3.19 (m, 2H), |
| | | | 2.81-2.63 (m, 3H), |
| | | | 2.58-2.49 (m, 1H), |
| | | | 2.39-2.29 (m, 1H), |
| | | | 2.22 (s, 3H), 2.13 (s, |
| | | | 6H), 1.97-1.90 (m, |
| | | | 1H), 1.38-1.29 (m, |
| | | | 1H). |
| 191 | N-[(6S)-6-(dimethylamino)-4- | 403 | 7.49 (d, 1H), 7.35 (d, |
| | methoxy-5,6,7,8- | | 1H), 6.73 (d, 1H), |
| | tetrahydronaphthalen-1-yl]-4- | | 6.67 (d, 1H), 3.73 (s, |
| | propylbenzenesulfonamide | | 3H), 2.86-2.64 (m, |
| | | | 5H), 2.59-2.49 (m, |
| | | | 1H), 2.33-2.23 (m, |
| | | | 1H), 2.17 (s, 6H), |
| | | | 1.76-1.70 (m, 1H), |
| | | | 1.60 (q, 2H), |
| | | | 1.13-1.08 (m, 1H), 0.87 (t, |
| | | | 3H). |
| 192 | 4-cyano-N-[(6S)-6-(dimethylamino)- | 386 | 8.04 (d, 2H), 7.76 (d, |
| | 4-methoxy-5,6,7,8- | | 2H), 6.66 (d, 1H), |
| | tetrahydronaphthalen-1- | | 6.62 (d, 1H), 3.72 (s, |
| | yl]benzenesulfonamide | | 3H), 2.74-2.63 (m, |
| | | | 3H), 2.59-2.25 (m, |
| | | | 2H), 2.20 (s, 6H), |
| | | | 1.85-1.77 (m, 1H), |
| | | | 1.28-1.19 (m, 1H). |
| 193 | 5-chloro-N-[(6S)-6-(dimethylamino)- | 401 | NA |
| | 4-methoxy-5,6,7,8- | | |
| | tetrahydronaphthalen-1-yl]thiophene- | | |
| | 2-sulfonamide | | |
| 194 | N-[(6S)-6-(dimethylamino)-4- | 411 | 8.20 (s, 1H), 8.12 (d, |
| | methoxy-5,6,7,8- | | 1H), 8.08 (d, 1H), |
| | tetrahydronaphthalen-1- | | 8.05 (d, 1H), 7.74 (d, |
| | yl]naphthalene-2-sulfonamide | | 1H), 7.70 (t, 1H), |
| | | | 7.65 (t, 1H), |
| | | | 6.62-6.58 (m, 2H), 3.69 (s, |
| | | | 3H), 2.79-2.74 (m, |
| | | | 1H), 2.73-2.63 (m, |
| | | | 2H), 2.37-2.21 (m, |
| | | | 2H), 2.13 (s, 6H), |
| | | | 1.76-1.71 (m, 1H), |
| | | | 1.14-1.09 (m, 1H). |

### Example 195 to 311

The following compounds were synthesized in an analogous method to example 173 (i)

| Ex- | MS | |
|---|---|---|
| ample | M + H | Name |
| 195 | 409 | 3-chloro-N-[(6S)-6-(dimethylamino)-4-methoxy- |
| | | 5,6,7,8-tetrahydronaphthalen-1-yl]-4- |
| | | methylbenzenesulfonamide |
| 196 | 453 | 4-bromo-N-[(6S)-6-(dimethylamino)-4-methoxy- |
| | | 5,6,7,8-tetrahydronaphthalen-1-yl]-2- |
| | | methylbenzenesulfonamide |
| 197 | 423 | 4-chloro-N-[(6S)-6-(dimethylamino)-4-methoxy- |
| | | 5,6,7,8-tetrahydronaphthalen-1-yl]-2,5- |
| | | dimethylbenzenesulfonamide |
| 198 | 415 | N-[(6S)-6-(dimethylamino)-4-methoxy-5,6,7,8- |
| | | tetrahydronaphthalen-1-yl]-2,3,4- |
| | | trifluorobenzenesulfonamide |
| 199 | 409 | 1-(3-chlorophenyl)-N-[(6S)-6-(dimethylamino)-4- |
| | | methoxy-5,6,7,8-tetrahydronaphthalen-1- |
| | | yl]methanesulfonamide |
| 200 | 415 | N-[(6S)-6-(dimethylamino)-4-methoxy-5,6,7,8- |
| | | tetrahydronaphthalen-1-yl]-2,4,5- |
| | | trifluorobenzenesulfonamide |
| 201 | 427 | 3-chloro-N-[(6S)-6-(dimethylamino)-4-methoxy- |
| | | 5,6,7,8-tetrahydronaphthalen-1-yl]-5-fluoro-2- |
| | | methylbenzenesulfonamide |
| 202 | 454 | N-[(6S)-6-(dimethylamino)-4-methoxy-5,6,7,8- |
| | | tetrahydronaphthalen-1-yl]-6-phenoxypyridine-3- |
| | | sulfonamide |
| 203 | 475 | 4-bromo-N-[(6S)-6-(dimethylamino)-4-methoxy- |
| | | 5,6,7,8-tetrahydronaphthalen-1-yl]-2,5- |
| | | difluorobenzenesulfonamide |
| 204 | 376 | N-[(6S)-6-(dimethylamino)-4-methoxy-5,6,7,8- |
| | | tetrahydronaphthalen-1-yl]-1-pyridin-2- |
| | | ylmethanesulfonamide |
| 205 | 437 | N-[(6S)-6-(dimethylamino)-4-methoxy-5,6,7,8- |
| | | tetrahydronaphthalen-1-yl]-1,1′-biphenyl-3- |
| | | sulfonamide |
| 206 | 401 | N-[(6S)-6-(dimethylamino)-4-methoxy-5,6,7,8- |
| | | tetrahydronaphthalen-1-yl]-1-benzofuran-2- |
| | | sulfonamide |
| 207 | 474 | 4-chloro-N¹-[(6S)-6-(dimethylamino)-4-methoxy- |
| | | 5,6,7,8-tetrahydronaphthalen-1-yl]benzene-1,3- |
| | | disulfonamide |
| 208 | 483 | N-[(6S)-6-(dimethylamino)-4-methoxy-5,6,7,8- |
| | | tetrahydronaphthalen-1-yl]-3-(2- |
| | | methoxyphenoxy)benzenesulfonamide |
| 209 | 467 | N-[(6S)-6-(dimethylamino)-4-methoxy-5,6,7,8- |
| | | tetrahydronaphthalen-1-yl]-4′-methoxy-1,1′-biphenyl- |
| | | 3-sulfonamide |
| 210 | 325 | N-[(6S)-6-(dimethylamino)-4-methoxy-5,6,7,8- |
| | | tetrahydronaphthalen-1-yl]cyclopropanesulfonamide |
| 211 | 429 | N-[(6S)-6-(dimethylamino)-4-methoxy-5,6,7,8- |
| | | tetrahydronaphthalen-1-yl]-4-fluoronaphthalene-1- |
| | | sulfonamide |
| 212 | 397 | N-[(6S)-6-(dimethylamino)-4-methoxy-5,6,7,8- |
| | | tetrahydronaphthalen-1-yl]-3,5- |
| | | difluorobenzenesulfonamide |
| 213 | 409 | N-[(6S)-6-(dimethylamino)-4-methoxy-5,6,7,8- |
| | | tetrahydronaphthalen-1-yl]-3-fluoro-4- |
| | | methoxybenzenesulfonamide |
| 214 | 529 | 1-[3-chloro-5-(trifluoromethyl)pyridin-2-yl]-N-[(6S)- |
| | | 6-(dimethylamino)-4-methoxy-5,6,7,8- |
| | | tetrahydronaphthalen-1-yl]-1H-pyrrole-2-sulfonamide |
| 215 | 502 | N-[(6S)-6-(dimethylamino)-4-methoxy-5,6,7,8- |
| | | tetrahydronaphthalen-1-yl]-5-[5- |
| | | (trifluoromethyl)isoxazol-3-yl]thiophene-2- |
| | | sulfonamide |
| 216 | 415 | N-[(6S)-6-(dimethylamino)-4-methoxy-5,6,7,8- |
| | | tetrahydronaphthalen-1-yl]-2,4,6- |
| | | trifluorobenzenesulfonamide |
| 217 | 434 | N-[(6S)-6-(dimethylamino)-4-methoxy-5,6,7,8- |
| | | tetrahydronaphthalen-1-yl]-5-isoxazol-5-ylthiophene- |
| | | 2-sulfonamide |
| 218 | 420 | N-[(6S)-6-(dimethylamino)-4-methoxy-5,6,7,8- |
| | | tetrahydronaphthalen-1-yl]-1-(3- |
| | | nitrophenyl)methanesulfonamide |
| 219 | 447 | N-[(6S)-6-(dimethylamino)-4-methoxy-5,6,7,8- |
| | | tetrahydronaphthalen-1-yl]-2-fluoro-5- |
| | | (trifluoromethyl)benzenesulfonamide |
| 220 | 459 | 2,3-dichloro-N-[(6S)-6-(dimethylamino)-4-methoxy- |
| | | 5,6,7,8-tetrahydronaphthalen-1-yl]-4- |
| | | methoxybenzenesulfonamide |
| 221 | 375 | N-[(6S)-6-(dimethylamino)-4-methoxy-5,6,7,8- |
| | | tetrahydronaphthalen-1-yl]-4- |
| | | methylbenzenesulfonamide |
| 222 | 454 | 5-(dimethylamino)-N-[(6S)-6-(dimethylamino)-4- |
| | | methoxy-5,6,7,8-tetrahydronaphthalen-1- |
| | | yl]naphthalene-1-sulfonamide |
| 223 | 406 | N-[(6S)-6-(dimethylamino)-4-methoxy-5,6,7,8- |
| | | tetrahydronaphthalen-1-yl]-2- |
| | | nitrobenzenesulfonamide |
| 224 | 406 | N-[(6S)-6-(dimethylamino)-4-methoxy-5,6,7,8- |
| | | tetrahydronaphthalen-1-yl]-4- |
| | | nitrobenzenesulfonamide |
| 225 | 361 | N-[(6S)-6-(dimethylamino)-4-methoxy-5,6,7,8- |
| | | tetrahydronaphthalen-1-yl]benzenesulfonamide |
| 226 | 451 | N-[(6S)-6-(dimethylamino)-4-methoxy-5,6,7,8- |
| | | tetrahydronaphthalen-1-yl]-3,4,5- |
| | | trimethoxybenzenesulfonamide |
| 227 | 375 | N-[(6S)-6-(dimethylamino)-4-methoxy-5,6,7,8- |
| | | tetrahydronaphthalen-1-yl]-1- |
| | | phenylmethanesulfonamide |
| 228 | 379 | N-[(6S)-6-(dimethylamino)-4-methoxy-5,6,7,8- |
| | | tetrahydronaphthalen-1-yl]-4- |
| | | fluorobenzenesulfonamide |
| 229 | 403 | N-[(6S)-6-(dimethylamino)-4-methoxy-5,6,7,8- |
| | | tetrahydronaphthalen-1-yl]-4- |
| | | isopropylbenzenesulfonamide |
| 230 | 487 | N-[(6S)-6-(dimethylamino)-4-methoxy-5,6,7,8- |
| | | tetrahydronaphthalen-1-yl]-4- |
| | | iodobenzenesulfonamide |
| 231 | 479 | 3-bromo-5-chloro-N-[(6S)-6-(dimethylamino)-4- |
| | | methoxy-5,6,7,8-tetrahydronaphthalen-1- |
| | | yl]thiophene-2-sulfonamide |
| 232 | 417 | 4-tert-butyl-N-[(6S)-6-(dimethylamino)-4-methoxy- |
| | | 5,6,7,8-tetrahydronaphthalen-1- |
| | | yl]benzenesulfonamide |
| 233 | 391 | N-[(6S)-6-(dimethylamino)-4-methoxy-5,6,7,8- |
| | | tetrahydronaphthalen-1-yl]-4- |
| | | methoxybenzenesulfonamide |
| 234 | 395 | 2-chloro-N-[(6S)-6-(dimethylamino)-4-methoxy- |
| | | 5,6,7,8-tetrahydronaphthalen-1- |
| | | yl]benzenesulfonamide |
| 235 | 418 | N-[4-({[(6S)-6-(dimethylamino)-4-methoxy-5,6,7,8- |
| | | tetrahydronaphthalen-1- |
| | | yl]amino}sulfonyl)phenyl]acetamide |
| 236 | 439 | 2-bromo-N-[(6S)-6-(dimethylamino)-4-methoxy- |
| | | 5,6,7,8-tetrahydronaphthalen-1- |
| | | yl]benzenesulfonamide |
| 237 | 500 | N-{[5-({[(6S)-6-(dimethylamino)-4-methoxy-5,6,7,8- |
| | | tetrahydronaphthalen-1-yl]amino}sulfonyl)thien-2- |
| | | yl]methyl}benzamide |
| 238 | 429 | N-[(6S)-6-(dimethylamino)-4-methoxy-5,6,7,8- |
| | | tetrahydronaphthalen-1-yl]-4- |
| | | (trifluoromethyl)benzenesulfonamide |
| 239 | 389 | N-[(6S)-6-(dimethylamino)-4-methoxy-5,6,7,8- |
| | | tetrahydronaphthalen-1-yl]-4- |
| | | ethylbenzenesulfonamide |
| 240 | 463 | 2-chloro-N-[(6S)-6-(dimethylamino)-4-methoxy- |
| | | 5,6,7,8-tetrahydronaphthalen-1-yl]-5- |
| | | (trifluoromethyl)benzenesulfonamide |
| 241 | 420 | N-[(6S)-6-(dimethylamino)-4-methoxy-5,6,7,8- |
| | | tetrahydronaphthalen-1-yl]-4-methyl-3- |
| | | nitrobenzenesulfonamide |
| 242 | 411 | N-[(6S)-6-(dimethylamino)-4-methoxy-5,6,7,8- |
| | | tetrahydronaphthalen-1-yl]naphthalene-1-sulfonamide |
| 243 | 440 | 4-chloro-N-[(6S)-6-(dimethylamino)-4-methoxy- |
| | | 5,6,7,8-tetrahydronaphthalen-1-yl]-3- |
| | | nitrobenzenesulfonamide |
| 244 | 429 | N-[(6S)-6-(dimethylamino)-4-methoxy-5,6,7,8- |
| | | tetrahydronaphthalen-1-yl]-3- |
| | | (trifluoromethyl)benzenesulfonamide |
| 245 | 395 | 4-chloro-N-[(6S)-6-(dimethylamino)-4-methoxy- |
| | | 5,6,7,8-tetrahydronaphthalen-1- |
| | | yl]benzenesulfonamide |
| 246 | 429 | 2,4-dichloro-N-[(6S)-6-(dimethylamino)-4-methoxy- |
| | | 5,6,7,8-tetrahydronaphthalen-1- |
| | | yl]benzenesulfonamide |
| 247 | 439 | N-[5-({[(6S)-6-(dimethylamino)-4-methoxy-5,6,7,8- |
| | | tetrahydronaphthalen-1-yl]amino}sulfonyl)-4-methyl- |
| | | 1,3-thiazol-2-yl]acetamide |
| 248 | 367 | N-[(6S)-6-(dimethylamino)-4-methoxy-5,6,7,8- |
| | | tetrahydronaphthalen-1-yl]thiophene-2-sulfonamide |
| 249 | 445 | 3,5-dichloro-N-[(6S)-6-(dimethylamino)-4-methoxy- |
| | | 5,6,7,8-tetrahydronaphthalen-1-yl]-2- |
| | | hydroxybenzenesulfonamide |
| 250 | 406 | N-[(6S)-6-(dimethylamino)-4-methoxy-5,6,7,8- |
| | | tetrahydronaphthalen-1-yl]-3- |
| | | nitrobenzenesulfonamide |
| 251 | 421 | N-[(6S)-6-(dimethylamino)-4-methoxy-5,6,7,8- |
| | | tetrahydronaphthalen-1-yl]-2,5- |
| | | dimethoxybenzenesulfonamide |
| 252 | 507 | N-[(6S)-6-(dimethylamino)-4-methoxy-5,6,7,8- |
| | | tetrahydronaphthalen-1-yl]-5- |
| | | (phenylsulfonyl)thiophene-2-sulfonamide |
| 253 | 444 | N-[(6S)-6-(dimethylamino)-4-methoxy-5,6,7,8- |
| | | tetrahydronaphthalen-1-yl]-5-pyridin-2-ylthiophene- |
| | | 2-sulfonamide |
| 254 | 386 | 2-cyano-N-[(6S)-6-(dimethylamino)-4-methoxy- |
| | | 5,6,7,8-tetrahydronaphthalen-1- |
| | | yl]benzenesulfonamide |
| 255 | 413 | 5-chloro-N-[(6S)-6-(dimethylamino)-4-methoxy- |
| | | 5,6,7,8-tetrahydronaphthalen-1-yl]-1,3-dimethyl-1H- |
| | | pyrazole-4-sulfonamide |
| 256 | 380 | N-[(6S)-6-(dimethylamino)-4-methoxy-5,6,7,8- |
| | | tetrahydronaphthalen-1-yl]-3,5-dimethylisoxazole-4- |
| | | sulfonamide |
| 257 | 365 | N-[(6S)-6-(dimethylamino)-4-methoxy-5,6,7,8- |
| | | tetrahydronaphthalen-1-yl]-1-methyl-1H-imidazole-4- |
| | | sulfonamide |
| 258 | 434 | N-[(6S)-6-(dimethylamino)-4-methoxy-5,6,7,8- |
| | | tetrahydronaphthalen-1-yl]-5-isoxazol-3-ylthiophene- |
| | | 2-sulfonamide |
| 259 | 425 | methyl 3-({[(6S)-6-(dimethylamino)-4-methoxy- |
| | | 5,6,7,8-tetrahydronaphthalen-1- |
| | | yl]amino}sulfonyl)thiophene-2-carboxylate |
| 260 | 429 | 2,6-dichloro-N-[(6S)-6-(dimethylamino)-4-methoxy- |
| | | 5,6,7,8-tetrahydronaphthalen-1- |
| | | yl]benzenesulfonamide |
| 261 | 397 | N-[(6S)-6-(dimethylamino)-4-methoxy-5,6,7,8- |
| | | tetrahydronaphthalen-1-yl]-2,6- |
| | | difluorobenzenesulfonamide |
| 262 | 420 | N-[(6S)-6-(dimethylamino)-4-methoxy-5,6,7,8- |
| | | tetrahydronaphthalen-1-yl]-2-methyl-5- |
| | | nitrobenzenesulfonamide |
| 263 | 375 | N-[(6S)-6-(dimethylamino)-4-methoxy-5,6,7,8- |
| | | tetrahydronaphthalen-1-yl]-3- |
| | | methylbenzenesulfonamide |
| 264 | 457 | 4-bromo-N-[(6S)-6-(dimethylamino)-4-methoxy- |
| | | 5,6,7,8-tetrahydronaphthalen-1-yl]-2- |
| | | fluorobenzenesulfonamide |
| 265 | 418 | N-[3-({[(6S)-6-(dimethylamino)-4-methoxy-5,6,7,8- |
| | | tetrahydronaphthalen-1- |
| | | yl]amino}sulfonyl)phenyl]acetamide |
| 266 | 420 | N-[(6S)-6-(dimethylamino)-4-methoxy-5,6,7,8- |
| | | tetrahydronaphthalen-1-yl]-2-methyl-4- |
| | | nitrobenzenesulfonamide |
| 267 | 395 | 3-chloro-N-[(6S)-6-(dimethylamino)-4-methoxy- |
| | | 5,6,7,8-tetrahydronaphthalen-1- |
| | | yl]benzenesulfonamide |
| 268 | 413 | 2-chloro-N-[(6S)-6-(dimethylamino)-4-methoxy- |
| | | 5,6,7,8-tetrahydronaphthalen-1-yl]-4- |
| | | fluorobenzenesulfonamide |
| 269 | 413 | 3-chloro-N-[(6S)-6-(dimethylamino)-4-methoxy- |
| | | 5,6,7,8-tetrahydronaphthalen-1-yl]-4- |
| | | fluorobenzenesulfonamide |
| 270 | 463 | 2-chloro-N-[(6S)-6-(dimethylamino)-4-methoxy- |
| | | 5,6,7,8-tetrahydronaphthalen-1-yl]-4- |
| | | (trifluoromethyl)benzenesulfonamide |
| 271 | 397 | N-[(6S)-6-(dimethylamino)-4-methoxy-5,6,7,8- |
| | | tetrahydronaphthalen-1-yl]-2,4- |
| | | difluorobenzenesulfonamide |
| 272 | 393 | N-[(6S)-6-(dimethylamino)-4-methoxy-5,6,7,8- |
| | | tetrahydronaphthalen-1-yl]-5-fluoro-2- |
| | | methylbenzenesulfonamide |
| 273 | 429 | 2,5-dichloro-N-[(6S)-6-(dimethylamino)-4-methoxy- |
| | | 5,6,7,8-tetrahydronaphthalen-1- |
| | | yl]benzenesulfonamide |
| 274 | 439 | 3-bromo-N-[(6S)-6-(dimethylamino)-4-methoxy- |
| | | 5,6,7,8-tetrahydronaphthalen-1- |
| | | yl]benzenesulfonamide |
| 275 | 409 | 3-chloro-N-[(6S)-6-(dimethylamino)-4-methoxy- |
| | | 5,6,7,8-tetrahydronaphthalen-1-yl]-2- |
| | | methylbenzenesulfonamide |
| 276 | 379 | N-[(6S)-6-(dimethylamino)-4-methoxy-5,6,7,8- |
| | | tetrahydronaphthalen-1-yl]-2- |
| | | fluorobenzenesulfonamide |
| 277 | 429 | N-[(6S)-6-(dimethylamino)-4-methoxy-5,6,7,8- |
| | | tetrahydronaphthalen-1-yl]-2- |
| | | (trifluoromethyl)benzenesulfonamide |
| 278 | 435 | 2,5-dichloro-N-[(6S)-6-(dimethylamino)-4-methoxy- |
| | | 5,6,7,8-tetrahydronaphthalen-1-yl]thiophene-3- |
| | | sulfonamide |
| 279 | 421 | N-[(6S)-6-(dimethylamino)-4-methoxy-5,6,7,8- |
| | | tetrahydronaphthalen-1-yl]-3,4- |
| | | dimethoxybenzenesulfonamide |
| 280 | 429 | 2,3-dichloro-N-[(6S)-6-(dimethylamino)-4-methoxy- |
| | | 5,6,7,8-tetrahydronaphthalen-1- |
| | | yl]benzenesulfonamide |
| 281 | 409 | 2-chloro-N-[(6S)-6-(dimethylamino)-4-methoxy- |
| | | 5,6,7,8-tetrahydronaphthalen-1-yl]-6- |
| | | methylbenzenesulfonamide |
| 282 | 429 | 3,4-dichloro-N-[(6S)-6-(dimethylamino)-4-methoxy- |
| | | 5,6,7,8-tetrahydronaphthalen-1- |
| | | yl]benzenesulfonamide |
| 283 | 429 | 3,5-dichloro-N-[(6S)-6-(dimethylamino)-4-methoxy- |
| | | 5,6,7,8-tetrahydronaphthalen-1- |
| | | yl]benzenesulfonamide |
| 284 | 445 | 5-bromo-N-[(6S)-6-(dimethylamino)-4-methoxy- |
| | | 5,6,7,8-tetrahydronaphthalen-1-yl]thiophene-2- |
| | | sulfonamide |
| 285 | 479 | 4-bromo-5-chloro-N-[(6S)-6-(dimethylamino)-4- |
| | | methoxy-5,6,7,8-tetrahydronaphthalen-1- |
| | | yl]thiophene-2-sulfonamide |
| 286 | 425 | 5-chloro-N-[(6S)-6-(dimethylamino)-4-methoxy- |
| | | 5,6,7,8-tetrahydronaphthalen-1-yl]-2- |
| | | methoxybenzenesulfonamide |
| 287 | 379 | N-[(6S)-6-(dimethylamino)-4-methoxy-5,6,7,8- |
| | | tetrahydronaphthalen-1-yl]-3- |
| | | fluorobenzenesulfonamide |
| 288 | 452 | N-[2-chloro-4-({[(6S)-6-(dimethylamino)-4-methoxy- |
| | | 5,6,7,8-tetrahydronaphthalen-1- |
| | | yl]amino}sulfonyl)phenyl]acetamide |
| 289 | 430 | N-[(6S)-6-(dimethylamino)-4-methoxy-5,6,7,8- |
| | | tetrahydronaphthalen-1-yl]-2-oxo-1,2,3,4- |
| | | tetrahydroquinoline-6-sulfonamide |
| 290 | 397 | N-[(6S)-6-(dimethylamino)-4-methoxy-5,6,7,8- |
| | | tetrahydronaphthalen-1-yl]-3,4- |
| | | difluorobenzenesulfonamide |
| 291 | 375 | N-[(6S)-6-(dimethylamino)-4-methoxy-5,6,7,8- |
| | | tetrahydronaphthalen-1-yl]-2- |
| | | methylbenzenesulfonamide |
| 292 | 389 | N-[(6S)-6-(dimethylamino)-4-methoxy-5,6,7,8- |
| | | tetrahydronaphthalen-1-yl]-2,5- |
| | | dimethylbenzenesulfonamide |
| 293 | 391 | N-[(6S)-6-(dimethylamino)-4-methoxy-5,6,7,8- |
| | | tetrahydronaphthalen-1-yl]-3- |
| | | methoxybenzenesulfonamide |
| 294 | 397 | N-[(6S)-6-(dimethylamino)-4-methoxy-5,6,7,8- |
| | | tetrahydronaphthalen-1-yl]-2,5- |
| | | difluorobenzenesulfonamide |
| 295 | 445 | 4-chloro-N-[(6S)-6-(dimethylamino)-4-methoxy- |
| | | 5,6,7,8-tetrahydronaphthalen-1-yl]naphthalene-1- |
| | | sulfonamide |
| 296 | 529 | N-[(6S)-6-(dimethylamino)-4-methoxy-5,6,7,8- |
| | | tetrahydronaphthalen-1-yl]-4-[2- |
| | | (phenylsulfonyl)ethyl]benzenesulfonamide |
| 297 | 445 | 8-chloro-N-[(6S)-6-(dimethylamino)-4-methoxy- |
| | | 5,6,7,8-tetrahydronaphthalen-1-yl]naphthalene-2- |
| | | sulfonamide |
| 298 | 472 | N-[4-({[(6S)-6-(dimethylamino)-4-methoxy-5,6,7,8- |
| | | tetrahydronaphthalen-1-yl]amino}sulfonyl)phenyl]- |
| | | 2,2,2-trifluoroacetamide |
| 299 | 501 | N-[(6S)-6-(dimethylamino)-4-methoxy-5,6,7,8- |
| | | tetrahydronaphthalen-1-yl]-2- |
| | | (phenylsulfonyl)benzenesulfonamide |
| 300 | 489 | 7-bromo-N-[(6S)-6-(dimethylamino)-4-methoxy- |
| | | 5,6,7,8-tetrahydronaphthalen-1-yl]naphthalene-1- |
| | | sulfonamide |
| 301 | 478 | 4-(1,3-benzoxazol-2-yl)-N-[(6S)-6-(dimethylamino)- |
| | | 4-methoxy-5,6,7,8-tetrahydronaphthalen-1- |
| | | yl]benzenesulfonamide |
| 302 | 425 | N-[(6S)-6-(dimethylamino)-4-methoxy-5,6,7,8- |
| | | tetrahydronaphthalen-1-yl]-4-methylnaphthalene-1- |
| | | sulfonamide |
| 303 | 445 | 5-chloro-N-[(6S)-6-(dimethylamino)-4-methoxy- |
| | | 5,6,7,8-tetrahydronaphthalen-1-yl]naphthalene-2- |
| | | sulfonamide |
| 304 | 462 | 4′-cyano-N-[(6S)-6-(dimethylamino)-4-methoxy- |
| | | 5,6,7,8-tetrahydronaphthalen-1-yl]-1,1′-biphenyl-2- |
| | | sulfonamide |
| 305 | 379 | N-[(6S)-6-(dimethylamino)-4-methoxy-5,6,7,8- |
| | | tetrahydronaphthalen-1-yl]-1,2-dimethyl-1H- |
| | | imidazole-4-sulfonamide |
| 306 | 367 | N-[(6S)-6-(dimethylamino)-4-methoxy-5,6,7,8- |
| | | tetrahydronaphthalen-1-yl]thiophene-3-sulfonamide |
| 307 | 431 | 2-chloro-N-[(6S)-6-(dimethylamino)-4-methoxy- |
| | | 5,6,7,8-tetrahydronaphthalen-1-yl]-4,5- |
| | | difluorobenzenesulfonamide |
| 308 | 439 | N-[(6S)-6-(dimethylamino)-4-methoxy-5,6,7,8- |
| | | tetrahydronaphthalen-1-yl]-4- |
| | | (methylsulfonyl)benzenesulfonamide |
| 309 | 431 | 4-chloro-N-[(6S)-6-(dimethylamino)-4-methoxy- |
| | | 5,6,7,8-tetrahydronaphthalen-1-yl]-2,5- |
| | | difluorobenzenesulfonamide |
| 310 | 437 | N-[(6S)-6-(dimethylamino)-4-methoxy-5,6,7,8- |
| | | tetrahydronaphthalen-1-yl]-1,1′-biphenyl-4- |
| | | sulfonamide |
| 311 | 405 | N-[(6S)-6-(dimethylamino)-4-methoxy-5,6,7,8- |
| | | tetrahydronaphthalen-1-yl]-2-methoxy-4- |
| | | methylbenzenesulfonamide |

### Example 312

### (i) N-[(6S)-4-methoxy-6-pyrrolidin-1-yl-5,6,7,8-tetrahydronaphthalen-1-yl]pyridine-3-sulfonamide

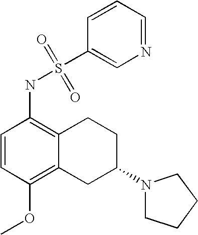

(6S)-4-Methoxy-6-pyrrolidin-1-yl-5,6,7,8-tetrahydronaphthalen-1-amine (60 mg, 0.24 mmol) and pyridine-3-sulfonylchloride (42 mg, 0.25 mmol) were suspended in dichlormethane (4 ml) and pyridine (0.15 ml) was added. The reaction mixture was stirred at ambient temperature over night. The solvent was removed and the residue was purified by preparative HPLC. The product was extracted from the LC-fractions using chloroform to give a solid (74 mg, 80%). ¹H NMR (400 MHz, DMSO-d₆) δ ppm 8.80 (1H, dd) 8.74 (1H, d) 7.95-8.00 (1H, m) 7.60 (1H, dd) 6.63-6.70 (2H, m) 3.71-3.74 (3H, m) 2.80 (1H, dd) 2.16-2.40 (4H, m) 1.80-1.90 (1H, m) 1.63-1.70 (4H, m) 1.19-1.35 (1H, m), m/z M+H388, M−H386.

### (ii) 1-[(2S)-8-methoxy-1,2,3,4-tetrahydronaphthalen-2-yl]pyrrolidine

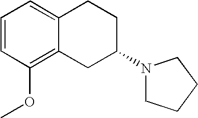

(2S)-8-Methoxy-1,2,3,4-tetrahydronaphthalen-2-ammonium chloride (21.3 g, 100 mmol) and 1,4-dibromobutane were suspended in DMF (200 ml), DIPEA (45 ml) was added and the reaction mixture was heated at 60° C. over night. The mixture was poured onto ice/water saturated with sodium hydrogencarbonate and extracted with EtOAc (×5). The combined organic layers were extracted with 1M hydrochloric acid. The acidic layer was treated with 5M aqueous sodium hydroxide until the pH was basic and the product was reextracted from the aqueous layer with EtOAc. The organic phase was dried over Na₂SO₄, filtered and the solvent was removed in vacuo. The product was isolated by chromatography on silica using a a gradient of CHCl₃/MeOH/NH₃ reaching from 0-10% of methanol containing ammonia (3%) yielding 6.5 g, 28%. ¹H NMR (400 MHz, DMSO-d₆) δ ppm 7.04 (1H, t) 6.72 (1H, d) 6.65 (1H, d) 3.75 (3H, s) 2.47-2.92 (7H, m) 2.27-2.44 (2H, m) 1.96-2.06 (1H, m) 1.62-1.73 (4H, m) 1.45-1.56 (1H, m), MS m/z M+H 232

### (iii) 1-[(2S)-5-Bromo-8-methoxy-1,2,3,4-tetrahydronaphthalen-2-yl]pyrrolidine

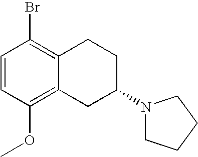

1-[(2S)-8-Methoxy-1,2,3,4-tetrahydronaphthalen-2-yl]pyrrolidine (example 312 (ii)) (3.08 g, 13.3 mmol) and sodium acetate (3.3 g, 40 mmol) were dissolved in acetic acid (80 ml). Bromine (0.69 ml, 13.3 ml) dissolved in acetic acid (40 ml) was added dropwise to the mixture over 5 hours. The solvent was removed in vacuo and dichloromethane was added. The organic phase was washed with 5M NaOH (aq) followed by brine, dried (Na₂SO₄), filtered and the solvent was evaporated. The residue was purified by chromatography on silica using a gradient of CHCl₃/MeOH/NH₃ reaching from 0-10% of methanol containing ammonia (3%) yielding an oil (2.41 g, 58%). ¹H NMR (400 MHz, DMSO-d₆) δ ppm 7.36 (1H, d) 6.75 (1H, d) 3.73-3.79 (3H, m) 2.84 (1H, dd) 2.75 (1H, dt) 2.44-2.63 (6H, m) 2.28-2.38 (1H, m) 1.97-2.07 (1H, m) 1.64-1.73 (4H, m) 1.54-1.64 (1H, m), MS m/z M+H 310, 312.

### (iv) (6S)—N-(Diphenylmethylene)-4-methoxy-6-pyrrolidin-1-yl-5,6,7,8-tetrahydronaphthalen-1-amine

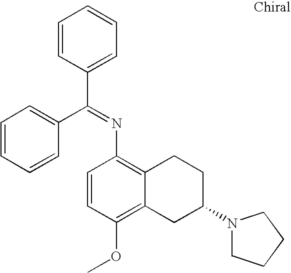

1-[(2S)-5-Bromo-8-methoxy-1,2,3,4-tetrahydronaphthalen-2-yl]pyrrolidine (2.4 g, 7.7 mmol), diphenylmethanimine (1.54 g, 8.5 mmol), tris(dibenzylideneacetone)dipalladium(0) (0.18 g, 0.2 mool), bis(2-diphenylphosphinophenyl)ether (0.21 g, 0.4 mmol) and sodium t-butoxide (2.2 g, 2.3 mmol) were mixed in toluene (40 ml) under argon atmosphere and heated at 100° C. for 3 hours. EtOAc and saturated aqueous sodium hydrogencarbonate were added. The organic layer was washed with saturated aqueous sodium hydrogencarbonate, dried over Na₂SO₄, filtered and the solvent was removed in vacuo. The residue was purified by chromatography on silica using a gradient of CHCl₃/MeOH/NH₃ reaching from 0-10% of methanol containing ammonia (3%) yielding 2.0 g (63%) of the title compound. ¹H NMR (400 MHz, DMSO-d₆) δ ppm 7.62-7.67 (2H, m) 7.41-7.54 (3H, m) 7.29-7.36 (3H, m) 7.07-7.15 (2H, m) 6.46 (1H, d) 6.16 (1H, d) 3.61-3.66 (3H, m) 2.71-2.90 (2H, m) 2.24-2.45 (4H, m) 1.99-2.09 (1H, m) 1.64-1.73 (4H, m) 1.42-1.55 (1H, m), MS m/z M+H 411

### (v) (6S)-4-Methoxy-6-pyrrolidin-1-yl-5,6,7,8-tetrahydronaphthalen-1-amine

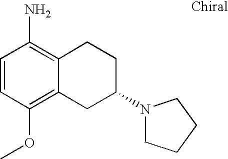

(6S)—N-(Diphenylmethylene)-4-methoxy-6-pyrrolidin-1-yl-5,6,7,8-tetrahydronaphthalen-1-amine (1.9 g, 4.6 mmol) was dissolved in THF (40 ml) and 1M hydrochloric acid (15 ml) was added. The reaction mixture was stirred vigorously over night. The reaction mixture was washed with heptane followed by EtOAc. The aqueous phase was made basic with 5M aqueous sodium hydroxide and extracted with dichloromethane. The organic phase was dried over Na₂SO₄, filtered and the solvent was evaporated. The crude product was purified by chromatography on silica using a gradient of CHCl₃/MeOH(NH₃ reaching from 0-10% of methanol containing ammonia (3%) yielding a solid (1.1 g, 99%).

¹H NMR (400 MHz, DMSO-d₆) δ ppm 6.52 (1H, d) 6.42 (1H, d) 4.25 (2H, s) 3.63 (3H, s) 2.84 (1H, dd) 2.46-2.62 (5H, m) 2.20-2.39 (3H, m) 2.00-2.08 (1H, m) 1.65-1.72 (4H, m) 1.44-1.55 (1H, m), MS APPI+M+H 247

### Example 313

### 3,5-Dichloro-N-[(6S)-4-methoxy-6-pyrrolidin-1-yl-5,6,7,8-tetrahydronaphthalen-1-yl]benzenesulfonamide

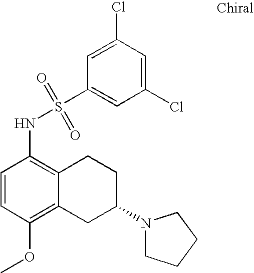

The product was prepared using the same method as in example 312 (i) and isolated as a solid (74 mg, 81%). ¹H NMR (400 MHz, DMSO-d₆) δ ppm 7.96 (1H, t) 7.56 (2H, d) 6.69 (1H, d) 6.64 (1H, d) 3.73 (3H, s) 2.83 (1H, dd) 2.52-2.63 (5H, m) 2.23-2.43 (3H, m) 1.85-1.95 (1H, m) 1.64-1.73 (4H, m) 1.28-1.41 (1H, m), MS m/z M+H 455, 457; M−H 453, 455.

### Example 314

### N-[(6S)-4-Methoxy-6-pyrrolidin-1-yl-5,6,7,8-tetrahydronaphthalen-1-yl]quinoline-8-sulfonamide

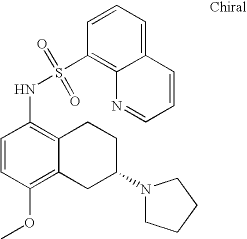

The product was prepared using the same method as in example 312 (i) and isolated as a solid (44 mg, 50%). ¹H NMR (400 MHz, DMSO-d₆) δ ppm 9.14 (1H, dd) 8.91 (1H, s) 8.59 (1H, dd) 8.30 (1H, dd) 8.16 (1H, dd) 7.77 (1H, dd) 7.69 (1H, t) 6.46 (1H, d) 6.30 (1H, d) 3.63 (3H, s) 2.68-2.80 (2H, m) 2.41-2.48 (4H, m) 2.25-2.36 (1H, m) 2.12-2.22 (1H, m) 1.79-1.90 (1H, m) 1.60-1.68 (4H, m) 1.22-1.34 (1H, m). MS m/z M+H 438; M−H 436

### Example 315

### N-[(6S)-4-Methoxy-6-pyrrolidin-1-yl-5,6,7,8-tetrahydronaphthalen-1-yl]naphthalene-1-sulfonamide

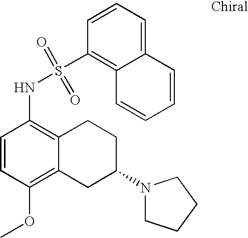

The product was prepared using the same method as in example 312 (i) and isolated as a solid (90 mg, 86%). ¹H NMR (600 MHz, CDCl₃) δ ppm 8.58-8.63 (1H, m) 8.13 (1H, d) 8.06 (1H, d) 7.92-7.97 (1H, m) 7.57-7.63 (2H, m) 7.47 (1H, t) 6.67 (1H, d) 6.46 (1H, d) 6.19 (1H, br. s.) 3.74 (3H, s) 2.95 (1H, dd) 2.60 (4H, br. s.) 2.54 (1H, dt) 2.21-2.35 (2H, m) 2.07-2.15 (1H, m) 1.86-1.93 (1H, m) 1.79 (4H, br. s.) MS m/z M+H 437; M−H435.

### Example 316

### (i) 4′-Chloro-N-[(6S)-4-methoxy-6-(methylamino)-5,6,7,8-tetrahydronaphthalen-1-yl]biphenyl-2-sulfonamide

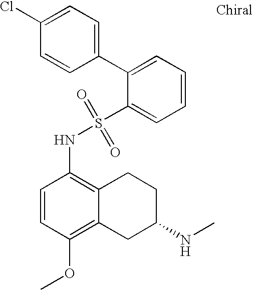

Ethyl ((2S)-5-{[(4′-chlorobiphenyl-2-yl)sulfonyl]amino}-8-methoxy-1,2,3,4-tetrahydronaphthalen-2-yl)carbamate (125 mg, 0.24 mmol) and lithium alumina hydride (36 mg, 0.96 mmol) were suspended in THF (5 ml). The reaction mixture was refluxed under argon atmosphere for 2 hours. The mixture was cooled to room temperature and carefully quenched with water. The mixture was extracted with dichloromethane (×1). The organic phase was dried (Na₂SO₄), filtered and the solvent was evaporated. The residue was purified by chromatography on silica using a a gradient of CHCl₃/MeOH/NH₃ reaching from 0-10% of methanol containing ammonia (3%) yielding the product (73 mg, 66%). ¹H NMR (400 MHz, CDCl₃) δ ppm 8.11 (1H, dd) 7.62 (1H, dt) 7.52 (1H, dt) 7.28-7.40 (4H, m) 6.50 (1H, d) 6.43 (1H, d) 3.73 (3H, s) 3.07 (1H, dd) 2.79-2.90 (1H, m) 2.27-2.61 (6H, m) 2.03-2.13 (1H, m) 1.53 (1H, none) 1.50-1.64 (1H, m) MS m/z M+H 457

### (ii) N-[(2S)-5-[(Diphenylmethylene)amino]-8-methoxy-1,2,3,4-tetrahydronaphthalen-2-yl]-2,2,2-trifluoroacetamide

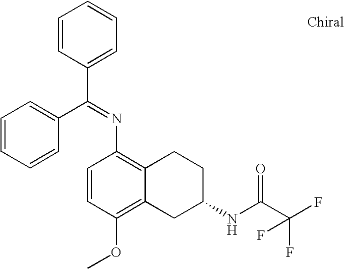

The title compound was prepared as described in Example 312 (iv) giving a solid (3.0 g, 53%). MS m/z M+H 453.

### (iii) N-[(2S)-5-amino-8-methoxy-1,2,3,4-tetrahydronaphthalen-2-yl]-2,2,2-trifluoroacetamide

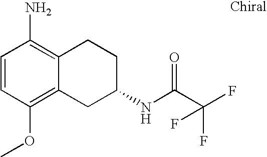

N-{(2S)-5-[(Diphenylmethylene)amino]-8-methoxy-1,2,3,4-tetrahydronaphthalen-2-yl}-2,2,2-trifluoroacetamide (3.0 g, 6.7 mmol) was dissolved in THF (50 ml) and hydrochloric acid (1 M, 22 ml) was added and the reaction mixture was stirred vigorously at ambient temperature over night. The mixture was concentrated in vacuo and the remains were neutralized with saturated sodium hydrogen carbonate solution. The mixture was extracted with EtOAc (×2), dichloromethane (×2) and chloroform (×2). The combined organic layers were dried (Na₂SO₄), filtered and the solvent was evaporated. The crude product was purified by chromatography on silica using a gradient of CHCl₃/MeOH/NH₃ reaching from 0-10% of methanol containing ammonia (3%) yielding a solid (1.1 g, 55%). MS m/z M+H 289

### (iv) N-[(6S)-6-Amino-4-methoxy-5,6,7,8-tetrahydronaphthalen-1-yl]-4′-chlorobiphenyl-2-sulfonamide

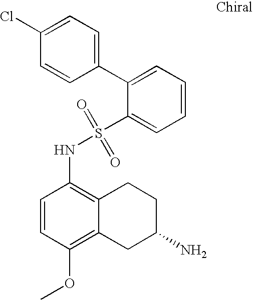

N-[(2S)-5-Amino-8-methoxy-1,2,3,4-tetrahydronaphthalen-2-yl]-2,2,2-trifluoroacetamide (280 mg, 0.97 mmol) and 4′-chlorobiphenyl-2-sulfonyl chloride (280 mg, 0.97 mmol) were dissolved in dichloromethane (6 ml). Pyridine (0.35 ml) was added and the reaction mixture was stirred over night. The mixture was washed with 1 M hydrochloric acid (×2) and saturated aqueous sodium hydrogen carbonate solution. The organic layer was dried (MgSO₄), filtered and the solvent was evaporated. The residue was dissolved in methanol (5 ml) and aqueous sodium hydroxide (2 M, 3 ml) was added. The mixture was stirred at ambient temperature over night. The mixture was concentrated in vacuo, acidified with hydrochloric acid, and made basic with saturated aqueous sodium hydrogen carbonate. The aqueous solution was extracted with dichloromethane (×2) and purified by column chromatography on silica The product was isolated by chromatography on silica using a a gradient of CHCl₃/MeOH/NH₃ reaching from 0-10% of methanol containing ammonia (3%) to give the title compound (30%). m/z ES+ M+H 443.

### (v) Ethyl ((2S)-5-{[(4′-chlorobiphenyl-2-yl)sulfonyl]amino}-8-methoxy-1,2,3,4-tetrahydronaphthalen-2-yl)carbamate

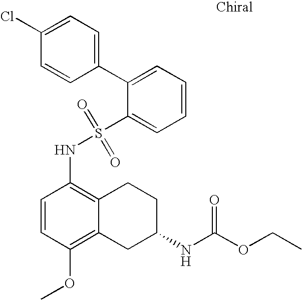

N-[(6S)-6-Amino-4-methoxy-5,6,7,8-tetrahydronaphthalen-1-yl]-4′-chlorobiphenyl-2-sulfonamide (172 mg, 0.39 mmol) and ethyl chloroformate (40 μl, 0.42 mmol) were dissolved in dichloromethane (5 ml). Pyridine (0.1 ml) was added and the reaction mixture was stirred for 4 hours at ambient temperature. The reaction mixture was washed with hydrochloric acid (1 M) and aqueous sodium hydrogen carbonate, dried (Na₂SO₄) and filtered. The solvent was removed in vacuo and the residue was purified on silica using heptane and EtOAc as eluents to give the title compound (130 mg, 96%). MS m/z ES− M−H513

### Example 317

### (i) 4′-Chloro-N-[(6S)-4-methoxy-6-(methylamino)-5,6,7,8-tetrahydronaphthalen-1-yl]-N-methylbiphenyl-2-sulfonamide

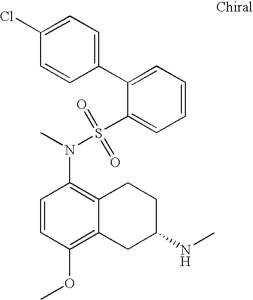

tert-Butyl {(2S)-5-[[(4′-chlorobiphenyl-2-yl)sulfonyl](methyl)amino]-8-methoxy-1,2,3,4-tetrahydronaphthalen-2-yl}methylcarbamate, (45 mg, 0.079 mmol) was dissolved in dichloromethane (6 ml) and TFA (0.5 ml) was added. The mixture was stirred vigorously at ambient temperature for 4 hours. The solvent was removed by evaporation and the residue was dissolved in methanol and loaded on a SCX column. The column was washed with methanol and the product was eluted in 0.7 M ammonia in methanol. The solvent was removed and the residue was purified by column chromatoghraphy on silica eluting with a a gradient of CHCl₃/MeOH/NH₃ reaching from 0-10% of methanol containing ammonia (3%) to give a solid (28 mg, 75%), ¹H NMR (400 MHz, CDCl₃, T=40° C., rotamers at lower temperature) δ ppm 7.94 (1H, dd) 7.57 (1H, dt) 7.46 (1H, t) 7.18-7.35 (5H, m) 6.36-6.52 (2H, m) 3.78 (3H, s) 3.02 (1H, dd) 2.63-2.94 (6H, m) 2.51 (3H, s) 2.31 (1H, dd) 1.96 (1H, br. s.) 1.34-1.48 (1H, m); MS m/z M+H 471

### (ii) Tert-Butyl ((2S)-5-{[(4′-chlorobiphenyl-2-yl)sulfonyl]amino}-8-methoxy-1,2,3,4-tetrahydronaphthalen-2-yl)methylcarbamate

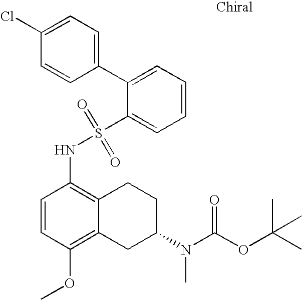

4′-Chloro-N-[(6S)-4-methoxy-6-(methylamino)-5,6,7,8-tetrahydronaphthalen-1-yl]biphenyl-2-sulfonamide (example 316 (i)) (56 mg, 0.12 mmol)) and di-tert-butyl dicarbonate (42 mg, 0.20 mmo) were dissolved in dichloromethane (5 ml). DIPEA (0.15 ml) was added and the mixture was stirred at ambient temperature for 2 hours. The mixture was washed with saturated aqueous sodium hydrogen carbonate solution (×2). The organic layer was dried (Na₂SO₄), filtered and the solvent was evaporated. The residue was purified by chromatography on silica using a gradient of heptane/ethyl acetate reaching from 0-100% of ethyl acetate to afford the product (48 mg, 70%). MS m/z M+H 555.

### (iii) Tert-Butyl [(2S)-5-[[(4′-chlorobiphenyl-2-yl)sulfonyl](methyl)amino]-8-methoxy-1,2,3,4-tetrahydronaphthalen-2-yl]methylcarbamate

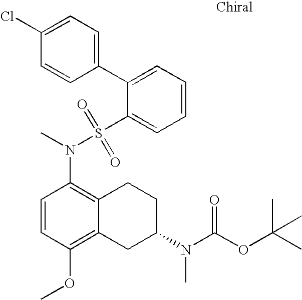

tert-Butyl ((2S)-5-{[(4′-chlorobiphenyl-2-yl)sulfonyl]amino}-8-methoxy-1,2,3,4-tetrahydronaphthalen-2-yl)methylcarbamate (46 mg, 0.083 mmol) and sodium hydride (60%, 14 mg, 0.35 mmol) were suspended in DMF (3 ml) and sonicated in an ultrasonic bath for 30 s. Iodomethane (40 mg, 0.29 mmol) was added and the reaction mixture was stirred for 2 hours. Water was added and the mixture was extracted with EtOAc (×2). The combined organic layers were washed with saturated aqueous sodium hydrogen carbonate, dried (Na₂SO₄) and the solvent was evaporated. The residue was purified by chromatography on silica using a gradient of heptane/ethyl acetate reaching from 0-100% of ethyl acetate to give the product (45 mg, 95%). m/z AP+M+H 571.

### Example 318

### N-[(6S)-4-Methoxy-6-pyrrolidin-1-yl-5,6,7,8-tetrahydronaphthalen-1-yl]naphthalene-1-sulfonamide

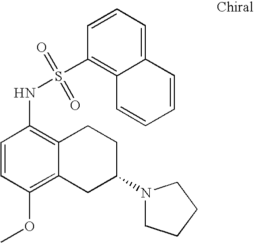

The title compound was prepared according to the method in example 312 to give a solid (86%). ¹H NMR (600 MHz, CDCl₃) δ ppm 8.58-8.62 (1H, m) 8.13 (1H, d) 8.06 (1H, d) 7.93-7.96 (1H, m) 7.58-7.63 (2H, m) 7.45-7.49 (1H, m) 6.67 (1H, d) 6.46 (1H, d) 3.74 (3H, s) 2.94 (1H, dd) 2.60 (4H, br. s.) 2.51-2.57 (1H, m) 2.22-2.34 (2H, m) 2.11 (1H, br. s.) 1.86-1.92 (1H, m) 1.79 (4H, br. s.) 1.17-1.27 (1H, m); MS m/z M+H⁺ 437, M−H⁻ 435.

### Example 319

### N-[(6S)-6-(Dimethylamino)-4-methoxy-5,6,7,8-tetrahydronaphthalen-1-yl]quinoline-8-sulfonamide

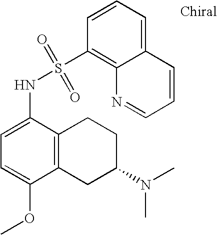

The title compound was prepared according to the method in example 312 to give a solid (47%).

¹H NMR (600 MHz, CD₃OD) δ ppm 9.14 (m, 1H) 8.52 (m, 1H) 8.24 (m, 1H) 8.19 (m, 1H) 7.73 (m, 1H) 7.65 (m, 1H) 6.42 (d, 1H) 6.26 (d, 1H) 3.69 (s, 3H) 3.20-3.34 (m, 2H) 3.11 (m, 1H) 2.70-2.86 (m, 7H) 2.58 (m, 1H) 2.22 (m, 1H) 1.58 (m, 1H)

MS m/z M+H⁺ 412

### Example 320

### 4′-Chloro-N-[(6S)-6-(dimethylamino)-4-methoxy-5,6,7,8-tetrahydronaphthalen-1-yl]biphenyl-2-sulfonamide

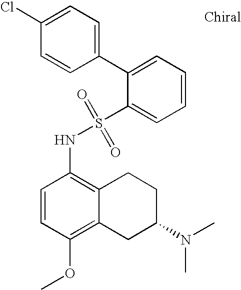

N-[(6S)-6-Amino-4-methoxy-5,6,7,8-tetrahydronaphthalen-1-yl]-4′-chlorobiphenyl-2-sulfonamide (example 316 (iv)) (52 mg, 0.12 mmol), formaldehyde (37% in water, 36 μl, 0.48 mmol) and acetic acid (0.1 ml) were dissolved in methanol (5 ml). The mixture was stirred at ambient temperature for 20 min. Sodium cyanoborohydride (30 mg, 0.48 mmol) was added and the mixture was stirred at ambient temperature for 2 hours. Saturated aqueous sodium hydrogen carbonate was added and the methanol was removed in vacuo. The mixture was extracted with dichloromethane. The organic phase was dried (Na₂SO₄), filtered and the solvent was evaporated. The residue was purified by chromatography on silica using a gradient of CHCl₃/MeOH/NH₃ reaching from 0-10% of methanol containing ammonia (3%) to give a solid (48 mg, 85%). ¹H NMR (400 MHz, CDCl₃) δ ppm 8.07-8.11 (1H, m) 7.59-7.64 (1H, m) 7.48-7.54 (1H, m) 7.27-7.38 (5H, m) 6.49 (1H, d) 6.43 (1H, d) 3.74 (3H, s) 2.91-2.99 (1H, m) 2.38-2.62 (9H, m) 2.23-2.35 (1H, m) 2.01-2.09 (1H, m) 1.39-1.51 (1H, m); MS m/z M+H+471, 473, M−H-469, 471.

### Example 321

### (i) 4′-Chloro-N-[(6S)-6-(dimethylamino)-4-methoxy-5,6,7,8-tetrahydronaphthalen-1-yl]-N-methylbiphenyl-2-sulfonamide

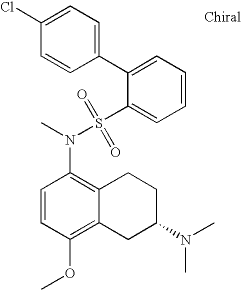

The title compound was prepared according to the method in example 320 to give the title compound as a solid (85%). ¹H NMR (400 MHz, CDCl₃) δ ppm 7.90-7.97 (1H, m) 7.55-7.61 (1H, m) 7.42-7.49 (1H, m) 7.27-7.36 (4H, m) 7.20 (1H, d) 6.34-6.50 (2H, m) 3.77-3.80 (3H, m) 2.92-3.05 (1H, m) 2.63-2.84 (4H, m) 2.34-2.54 (9H, m) 1.97-2.10 (1H, m) 1.33-1.47 (1H, m); APPI-MS m/z M+H⁺ 485, 487.

### (ii) N-[(6S)-6-Amino-4-methoxy-5,6,7,8-tetrahydronaphthalen-1-yl]-4′-chloro-N-methylbiphenyl-2-sulfonamide

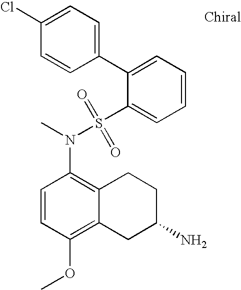

The title compound was prepared according to the method in example 317 (i) to give the title compound. The product obtained from the SCX column was used in the next step. APPI-MS m/z M+H⁺ 457, 459.

### (iii) tert-Butyl {(2S)-5-[[(4′-chlorobiphenyl-2-yl)sulfonyl](methyl)amino]-8-methoxy-1,2,3,4-tetrahydronaphthalen-2-yl}carbamate

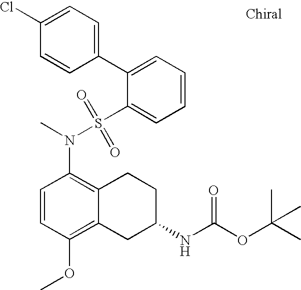

The title compound was prepared according to the method in example 317 (iii). ESI-MS m/z M+NH₃⁺ 574, 576.

### Pharmacology

Method for [125I]SB258585 binding to rat striatal 5HT6 receptors

### Materials

[125I]SB258585 (1) with specific activity 2000 Ci/mmol was purchased from Amersham Biosciences Europe GmbH, Freiburg, Germany. Other chemicals were purchased from commercial sources and were of analytical grade.

### Preparation of Membranes:

Striatal tissue from adult rats (Sprague-Dawley, 320-370 g, B & K Sweden) were dissected out, weighed and homogenized in buffer containing 50 mM Tris-HCl, 4 mM MgCl2, 1 mM EDTA, 10 μM pargyline and protease inhibitor (Complete, Roche Diagnostics) pH 7.4 using an Ultra-Turrax T8 (IKA Labortechnik, Germany). The tissue homogenate was centrifuged at 48 000×g for 10 min and the pellet was resuspended and recentrifuged as above. The final membranes were diluted in buffer to a concentration of 60 mg original wet weight (w.w.) per ml and stored in aliquots at −70° C.

### Radioligand Binding Assays:

Saturation binding studies were carried out in duplicate with 1-3 mg w.w. per tube in 0.5 ml buffer (50 mM Tris, 4 mM MgCl2, 100 mM NaCl, 1 mM EDTA, 5 mM ascorbate and 10 μM pargyline at pH 7.4), 0.2 nM [125I]SB258585 and unlabelled SB258585 to give a final concentration range of 0.23-20 nM (12 conc.). Non-specific binding was determined in the presence of 10 μM methiothepin. In the competition experiments 0.8-2 mg w.w. per tube and a radioligand concentration of 0.5-1 nM were used with 7 concentrations of the competing drug pre-dissolved in DMSO and diluted in buffer. The assays were incubated for 1-3 hours at room temperature, and terminated by rapid filtration through Whatman GF/B filters pretreated with 0.3% polyethyleneimine using a Brandel cell harvester. The radioactivity was determined in a Packard Tri-Carb 2900TR liquid scintillation counter. Data were analyzed by non-linear regression analyses using PRISM 4.00 (GraphPad Software Inc., San Diego, Calif.).

Hirst, W. D., Minton, J. A. L., Bromidge, S. M., Moss, S. F., Latter, A., Riley, G., Routledge, C., Middlemiss, D. N. & Price, G. W. (2000). Characterization of [125I]-SB-258585 binding to human recombinant and native 5HT6 receptors in rat, pig and human brain tissue. Br. J. Pharmacol., 130, 1597-1605.

### Results

Typical IC₅₀ values as measured in the assays described above are 1 μM or less. In one aspect of the invention the IC₅₀ is below 500 nM. In another aspect of the invention the IC₅₀ is below 50 nM. In a further aspect of the invention the IC₅₀ is below 10 nM.

TABLE 1
Specimen results from assay.

| Example no | Kᵢ (nM) |
|---|---|
| 122 | 2.0 ± 0.6 |
| 24 | 42 ± 27 |
| 3 | 7.5 ± 3.6 |
| 135 | 49 ± 16 |
| 176 | 13 ± 5.9 |
| 181 | 19 ± 0.8 |
| 313 | 290 ± 190 |
| 170 | 74 |

1. A compound having the formula I 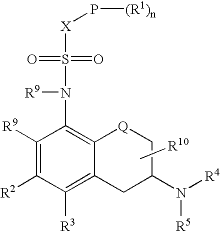
wherein:
P is C₆₋₁₀arylC₀₋₆alkyl, C₅₋₁₁heteroarylC₀₋₆alkyl, C₃₋₇cycloalkylC₀₋₆alkyl, C₃₋₇heterocycloalkylC₀₋₆alkyl, or C₂₋₁₀alkyl;
R¹ is hydrogen, hydroxy, halogen, C₁₋₁₀alkyl, C₂₋₁₀alkenyl, C₂₋₁₀alkynyl, C₁₋₁₀alkoxy, N(R¹¹)₂, C₆₋₁₀arylC₀₋₆alkyl, C₅₋₁₁heteroarylC₀₋₆alkyl, C₁₋₆haloalkyl, C₁₋₆haloalkylO, R⁷OC₀₋₆alkyl, cyano, NO₂, SR⁷, R⁷SO₂C₀₋₄alkyl, SOR⁷, R⁷CON(R⁸)C₀₋₄alkyl, N(R⁸)SO₂R⁷, COR⁷, COOR⁸, OSO₂R⁷, (R⁸)₂NCOC₀₋₆alkyl, oxo, or SO₂N(R⁸)₂;
n is 0, 1, 2, 3, 4 or 5;
X is a single bond, C₁₋₃alkyl, or NR⁵, or X is N in a heteroalkyl or C₅₋₁₁heteroaryl; or
N, SO₂, X and P form together a C₈₋₁₁heteroaryl or C₈₋₁₁bicycloheteroalkyl;
Q is CH or O;
R² is hydrogen, hydroxy, halogen, C₁₋₁₀alkyl, C₂₋₁₀alkenyl, C₂₋₁₀alkynyl, C₁₋₁₀alkoxy, N(R¹¹)₂, C₆₋₁₀arylC₀₋₆alkyl, C₅₋₆heteroarylC₀₋₆alkyl, C₁₋₆haloalkyl, C₁₋₆haloalkylO, R⁷OC₀₋₆alkyl, cyano, SR⁷SO₂R⁸SOR⁷, NCOR⁷, NR⁸SO₂R⁷, COR⁷, COOR⁷, OSO₂R⁷, CON(R⁸)₂, or SO₂N(R⁸)₂;
R³ is hydrogen, hydroxy, halogen, C₁₋₁₀alkyl, C₂₋₁₀alkenyl, C₂₋₁₀alkynyl, C₁₋₁₀alkoxy, N(R¹¹)₂, C₆₋₁₀arylC₀₋₆alkyl, C₅₋₆heteroarylC₀₋₆alkyl, C₁₋₆haloalkyl, C₁₋₆haloalkylO, R⁷OC₀₋₆alkyl, cyano, SR⁷, SO₂R⁷, SOR⁷, N(R⁸)COR⁷, N(R⁸)SO₂R⁷, COR⁷, COO R⁷, OSO₂R⁷, CON(R⁸)₂, or SO₂N(R⁸)₂;
R⁴ and R⁵ are each independently selected from hydrogen, C₁₋₅alkyl, C₁₋₅haloalkyl, C₂₋₅alkenyl, C₂₋₅alkynyl, C₃₋₆cycloalkyl, C₅₋₆arylC₁₋₂alkyl, and C₅₋₆heteroarylC₁₋₂alkyl, wherein each R⁴ and R⁵ is optionally and independently substituted by one or more groups independently selected from halogen, hydroxyl, cyano, and C₁₋₅alkoxy, or
R⁴ and R⁵ form together C₃₋₇heterocycloalkyl, wherein R⁴ and R⁵ are each optionally substituted by one or more groups independently selected from hydrogen, halogen, C₁₋₆alkyl, C₁₋₆haloalkyl, C₅₋₆aryl, C₅₋₆heteroaryl, COR¹², SO₂R¹², OR¹², cyano, SO₂N(R¹¹)₂, and oxo substituted on β or γ position;
R⁶ is hydrogen, C₁₋₆alkyl, C₃₋₆cycloakyl, R⁷OC₁₋₆alkyl, C₁₋₆haloalkyl, C₁₋₆cyanoalkyl, (R¹¹)₂NCOC₀₋₆alkyl, or R¹²SO₂C₁₋₆alkyl;
R⁷ is C₁₋₁₀alkyl, C₁₋₆haloalkyl, C₆₋₁₀arylC₀₋₆alkyl, C₅₋₆heteroarylC₀₋₆alkyl, C₃₋₇cycloalkylC₀₋₆alkyl, or C₁₋₆alkoxyC₆₋₁₀aryl;
R⁸ is hydrogen, C₁₋₁₀alkyl, C₃₋₇cycloalkylC₀₋₆alkyl, C₆₋₁₀arylC₀₋₆alkyl, C₁₋₆haloalkyl, or C₅₋₆heteroarylC₀₋₆alkyl, or
R⁷ and R⁸ form together a C₅₋₆heteroaryl or C₃₋₇heterocycloalkyl;
wherein each R¹, R⁷, and any R⁸ aryl and each R¹, R⁷, and R⁸ heteroaryl is optionally and independently substituted by one or more groups independently selected from hydrogen, halogen, hydroxy, C₁₋₆haloalkyl, cyano, alkyl, OR¹², oxo, C₁₋₅alkoxy, SOR¹², SR¹¹, CON(R¹¹)₂, N(R¹¹)COR¹², SO₂R¹², N(R¹¹)₂, and COR¹²;
R⁹ is hydrogen, halogen, hydroxy, C₁₋₆alkoxy, C₁₋₆haloalkoxy, C₁₋₆haloalkyl, C₁₋₆alkyl, or COR¹²;
R¹⁰ is hydrogen, C₁₋₆alkyl, C₁₋₆alkoxy, or C₁₋₆haloalkyl;
R¹¹ is hydrogen, C₁₋₆alkyl or C₁₋₆haloalkyl; and
R¹² is C₁₋₆alkyl or C₁₋₆haloalkyl, or
R¹¹ and R¹² form together a C₃₋₇cycloalkyl or C₃₋₇heterocycloalkyl, wherein each R¹¹ and R¹² is optionally and independently substituted by one or more groups independently selected from hydrogen, halogen, hydroxy, cyano, C₁₋₃alkyl, C₁₋₃alkoxy, and C₁₋₃haloalkyl;
or salts, solvates, or solvated salts thereof.

2. The compound according to claim 1, wherein:
P is C₆₋₁₀arylC₀₋₆alkyl, C₅₋₁₁heteroarylC₀₋₆alkyl, C₃₋₇cycloalkylC₀₋₆alkyl, or C₂₋₁₀alkyl;
R¹ is hydrogen, hydroxy, halogen, C₁₋₁₀alkyl, C₁₋₁₀alkoxy, C₆₋₁₀arylC₀₋₆alkyl, C₅₋₁₁heteroarylC₀₋₆alkyl, C₁₋₆haloalkyl, R⁷OC₀₋₆alkyl, NO₂, R⁷SO₂C₀₋₄alkyl, R⁷CON(R⁸)C₀₋₄alkyl, COR⁷, or SO₂N(R⁸)₂;
n is 0, 1, 2, 3, or 4;
X is a single bond or NR⁶;
Q is CH or O;
R² is hydrogen;
R³ is halogen or C₁₋₁₀alkoxy;
R⁴ and R⁵ are each independently selected from hydrogen or C₁₋₅alkyl, or
R⁴ and R⁵ form together C₃₋₇heterocycloalkyl;
R⁶ is hydrogen;
R⁷ is C₁₋₁₀alkyl, C₁₋₆haloalkyl, C₆₋₁₀arylC₀₋₆alkyl, C₃₋₇cycloalkylC₀₋₆alkyl, or C₁₋₆alkoxyC₆₋₁₀aryl;
R⁸ is a hydrogen, C₁₋₁₀alkyl, C₆₋₁₀arylC₀₋₆alkyl, or C₁₋₆haloalkyl;
wherein each R¹, R⁷, and R⁸ aryl and each R¹, R⁷, and R⁸ heteroaryl is optionally and independently substituted by one or more groups independently selected from hydrogen, halogen, C₁₋₆haloalkyl, cyano, C₁₋₅alkoxy, or SR¹¹;
R⁹ is hydrogen; and
R¹⁰ is hydrogen;
or salts, solvates, or solvated salts thereof.

3. The compound according to claim 1, wherein P is phenyl, naftyl, pyridinyl, pyrrolyl, benzodioxanyl, methylpyridinyl, benzofuryl, thiophenyl, thioimidazolyl, benzothiaimidazolyl, benzofurazanyl, thiazolylpyrazolyl, imidazolyl, methylphenyl, indolinyl, benzopyrrolidinyl, quinoline, isoquinoline, thiazolyl, imidazothiazolyl, furyl, ethyl, cyclopropyl, thienyl, ethylnaphtyl, chromane, or indane.

4. The compound according to claim 1, wherein R¹ is hydrogen, chloro, fluoro, bromo, iodo, methyl, ethyl, i-propyl, n-propyl, n-butyl, tert-butyl, phenoxy, methoxy, ethoxy, propoxy, pyridinyl, isooxazole, benzooxazolyl, thiophenyl, methylCON, phenylNCOmethyl, phenylSO₂-ethyl, nitro, phenylSO₂, methylSO₂, NH₂SO₂, phenyl, cyano, COOmethyl, pyrimidyl, pyrazolyl, COmethyl, hydroxy, fluoromethyl, difluoromethyl, trifluoromethyl, fluoromethoxy, difluoromethoxy, or trifluoromethoxy.

5. The compound according to claim 1, wherein R³ is halogen, methoxy, ethoxy, propoxy, fluoromethyl, difluoromethyl, trifluoromethyl, fluoromethoxy, difluoromethoxy, or trifluoromethoxy.

6. The compound according to claim 1, wherein X is a bond, NH, indol, indoline, tetrahydroquinoline, tetrahydroisoquinoline, benzoxazepine, isoindoline, or benzazepine.

7. The compound according to claim 1, wherein R⁴ and R⁵ are each independently selected from hydrogen, methyl, ethyl, i-propyl, n-propyl, fluoroethyl, and pyrrolidine.

8. A compound according to claim 1, wherein said compound is selected from
(3R)-5-Methoxy-N,N-dimethyl-8-[(phenylsulfonyl)amino]chroman-3-ammonium acetate,
(3R)-8-{[(4-Chlorophenyl)sulfonyl]amino}-5-methoxy-N,N-dimethylchroman-3-ammonium acetate,
3-Bromo-N-[(3R)-3-(dimethylamino)-5-methoxy-3,4-dihydro-2H-chromen-8-yl]benzenesulfonamide,
N-[(3R)-3-(Dimethylamino)-5-methoxy-3,4-dihydro-2H-chromen-8-yl]biphenyl-4-sulfonamide,
N-[(3R)-3-(dimethylamino)-5-methoxy-3,4-dihydro-2H-chromen-8-yl]-2-methoxy-4-methylbenzenesulfonamide,
6-chloro-N-[(3R)-3-(dimethylamino)-5-methoxy-3,4-dihydro-2H-chromen-8-yl]imidazo[2,1-b][1,3]thiazole-5-sulfonamide,
N-[(3R)-3-(dimethylamino)-5-methoxy-3,4-dihydro-2H-chromen-8-yl]-2-(methylsulfonyl)benzenesulfonamide,
5-chloro-N-[(3R)-3-(dimethylamino)-5-methoxy-3,4-dihydro-2H-chromen-8-yl]-3-methyl-1-benzothiophene-2-sulfonamide,
7-chloro-N-[(3R)-3-(dimethylamino)-5-methoxy-3,4-dihydro-2H-chromen-8-yl]-2,1,3-benzoxadiazole-4-sulfonamide,
N-[(3R)-3-(dimethylamino)-5-methoxy-3,4-dihydro-2H-chromen-8-yl]-3-(trifluoromethoxy)benzenesulfonamide,
N-[(3R)-3-(dimethylamino)-5-methoxy-3,4-dihydro-2H-chromen-8-yl]-2,3-dihydro-1,4-benzodioxine-6-sulfonamide,
3-(2-chlorophenoxy)-N-[(3R)-3-(dimethylamino)-5-methoxy-3,4-dihydro-2H-chromen-8-yl]benzenesulfonamide,
4,5-dichloro-N-[(3R)-3-(dimethylamino)-5-methoxy-3,4-dihydro-2H-chromen-8-yl]thiophene-2-sulfonamide,
N-[(3R)-3-(dimethylamino)-5-methoxy-3,4-dihydro-2H-chromen-8-yl]-2-(1-naphthyl)ethanesulfonamide,
4-chloro-N-[(3R)-3-(dimethylamino)-5-methoxy-3,4-dihydro-2H-chromen-8-yl]naphthalene-1-sulfonamide,
4′-cyano-N-[(3R)-3-(dimethylamino)-5-methoxy-3,4-dihydro-2H-chromen-8-yl]-1,1′-biphenyl-2-sulfonamide,
N-[(3R)-3-(dimethylamino)-5-methoxy-3,4-dihydro-2H-chromen-8-yl]-3-(trifluoromethyl)benzenesulfonamide,
N-[(3R)-3-(dimethylamino)-5-methoxy-3,4-dihydro-2H-chromen-8-yl]-5-pyridin-2-ylthiophene-2-sulfonamide,
N-[3-({[(3R)-3-(dimethylamino)-5-methoxy-3,4-dihydro-2H-chromen-8-yl]amino}sulfonyl)phenyl]acetamide,
1-acetyl-5-bromo-N-[(3R)-3-(dimethylamino)-5-methoxy-3,4-dihydro-2H-chromen-8-yl]indoline-6-sulfonamide,
4-cyano-N-[(3R)-3-(dimethylamino)-5-methoxy-3,4-dihydro-2H-chromen-8-yl]benzenesulfonamide,
N-[(3R)-3-(dimethylamino)-5-methoxy-3,4-dihydro-2H-chromen-8-yl]-4-propylbenzenesulfonamide,
N-[(3R)-3-(dimethylamino)-5-methoxy-3,4-dihydro-2H-chromen-8-yl]naphthalene-2-sulfonamide,
N-[(3R)-3-(dimethylamino)-5-methoxy-3,4-dihydro-2H-chromen-8-yl]-4-methylbenzenesulfonamide,
4-bromo-N-[(3R)-3-(dimethylamino)-5-methoxy-3,4-dihydro-2H-chromen-8-yl]benzenesulfonamide,
3-bromo-5-chloro-N-[(3R)-3-(dimethylamino)-5-methoxy-3,4-dihydro-2H-chromen-8-yl]thiophene-2-sulfonamide,
4-tert-butyl-N-[(3R)-3-(dimethylamino)-5-methoxy-3,4-dihydro-2H-chromen-8-yl]benzenesulfonamide,
N-[(3R)-3-(dimethylamino)-5-methoxy-3,4-dihydro-2H-chromen-8-yl]-4-methoxybenzenesulfonamide,
2-chloro-N-[(3R)-3-(dimethylamino)-5-methoxy-3,4-dihydro-2H-chromen-8-yl]benzenesulfonamide,
N-[4-({[(3R)-3-(dimethylamino)-5-methoxy-3,4-dihydro-2H-chromen-8-yl]amino}sulfonyl)phenyl]acetamide,
2-bromo-N-[(3R)-3-(dimethylamino)-5-methoxy-3,4-dihydro-2H-chromen-8-yl]benzenesulfonamide,
N-{[5-({[(3R)-3-(dimethylamino)-5-methoxy-3,4-dihydro-2H-chromen-8-yl]amino}sulfonyl)thien-2-yl]methyl}benzamide,
N-[(3R)-3-(dimethylamino)-5-methoxy-3,4-dihydro-2H-chromen-8-yl]-4-(trifluoromethyl)benzenesulfonamide,
N-[(3R)-3-(dimethylamino)-5-methoxy-3,4-dihydro-2H-chromen-8-yl]-4-ethylbenzenesulfonamide,
N-[(3R)-3-(dimethylamino)-5-methoxy-3,4-dihydro-2H-chromen-8-yl]-2-nitrobenzenesulfonamide,
2-chloro-N-[(3R)-3-(dimethylamino)-5-methoxy-3,4-dihydro-2H-chromen-8-yl]-5-(trifluoromethyl)benzenesulfonamide,
N-[(3R)-3-(dimethylamino)-5-methoxy-3,4-dihydro-2H-chromen-8-yl]-4-methyl-3-nitrobenzenesulfonamide,
N-[(3R)-3-(dimethylamino)-5-methoxy-3,4-dihydro-2H-chromen-8-yl]naphthalene-1-sulfonamide,
4-chloro-N-[(3R)-3-(dimethylamino)-5-methoxy-3,4-dihydro-2H-chromen-8-yl]-3-nitrobenzenesulfonamide,
4-chloro-N-[(3R)-3-(dimethylamino)-5-methoxy-3,4-dihydro-2H-chromen-8-yl]benzenesulfonamide,
2,4-dichloro-N-[(3R)-3-(dimethylamino)-5-methoxy-3,4-dihydro-2H-chromen-8-yl]benzenesulfonamide,
N-[5-({[(3R)-3-(dimethylamino)-5-methoxy-3,4-dihydro-2H-chromen-8-yl]amino}sulfonyl)-4-methyl-1,3-thiazol-2-yl]acetamide,
N-[(3R)-3-(dimethylamino)-5-methoxy-3,4-dihydro-2H-chromen-8-yl]thiophene-2-sulfonamide,
N-[(3R)-3-(dimethylamino)-5-methoxy-3,4-dihydro-2H-chromen-8-yl]-4-nitrobenzenesulfonamide,
3,5-dichloro-N-[(3R)-3-(dimethylamino)-5-methoxy-3,4-dihydro-2H-chromen-8-yl]-2-hydroxybenzenesulfonamide,
N-[(3R)-3-(dimethylamino)-5-methoxy-3,4-dihydro-2H-chromen-8-yl]-3-nitrobenzenesulfonamide,
N-[(3R)-3-(dimethylamino)-5-methoxy-3,4-dihydro-2H-chromen-8-yl]-2,5-dimethoxybenzenesulfonamide,
4,5-dibromo-N-[(3R)-3-(dimethylamino)-5-methoxy-3,4-dihydro-2H-chromen-8-yl]thiophene-2-sulfonamide,
5-bromo-N-[(3R)-3-(dimethylamino)-5-methoxy-3,4-dihydro-2H-chromen-8-yl]-2-methoxybenzenesulfonamide,
N-[(3R)-3-(dimethylamino)-5-methoxy-3,4-dihydro-2H-chromen-8-yl]-5-(phenylsulfonyl)thiophene-2-sulfonamide,
N-[(3R)-3-(dimethylamino)-5-methoxy-3,4-dihydro-2H-chromen-8-yl]-5-[1-methyl-5-(trifluoromethyl)-1H-pyrazol-3-yl]thiophene-2-sulfonamide,
2-cyano-N-[(3R)-3-(dimethylamino)-5-methoxy-3,4-dihydro-2H-chromen-8-yl]benzenesulfonamide,
5-chloro-N-[(3R)-3-(dimethylamino)-5-methoxy-3,4-dihydro-2H-chromen-8-yl]-1,3-dimethyl-1H-pyrazole-4-sulfonamide,
N-[(3R)-3-(dimethylamino)-5-methoxy-3,4-dihydro-2H-chromen-8-yl]benzenesulfonamide,
N-[(3R)-3-(dimethylamino)-5-methoxy-3,4-dihydro-2H-chromen-8-yl]-3,5-dimethylisoxazole-4-sulfonamide,
N-[(3R)-3-(dimethylamino)-5-methoxy-3,4-dihydro-2H-chromen-8-yl]-1-methyl-1H-imidazole-4-sulfonamide,
N-[(3R)-3-(dimethylamino)-5-methoxy-3,4-dihydro-2H-chromen-8-yl]-5-isoxazol-3-ylthiophene-2-sulfonamide,
methyl 3-({[(3R)-3-(dimethylamino)-5-methoxy-3,4-dihydro-2H-chromen-8-yl]amino}sulfonyl)thiophene-2-carboxylate,
N-[(3R)-3-(dimethylamino)-5-methoxy-3,4-dihydro-2H-chromen-8-yl]-4-phenoxybenzenesulfonamide,
N-[(3R)-3-(dimethylamino)-5-methoxy-3,4-dihydro-2H-chromen-8-yl]-3,5-bis(trifluoromethyl)benzenesulfonamide,
2,6-dichloro-N-[(3R)-3-(dimethylamino)-5-methoxy-3,4-dihydro-2H-chromen-8-yl]benzenesulfonamide,
N-[(3R)-3-(dimethylamino)-5-methoxy-3,4-dihydro-2H-chromen-8-yl]-2,6-difluorobenzenesulfonamide,
N-[(3R)-3-(dimethylamino)-5-methoxy-3,4-dihydro-2H-chromen-8-yl]-2-methyl-5-nitrobenzenesulfonamide,
N-[(3R)-3-(dimethylamino)-5-methoxy-3,4-dihydro-2H-chromen-8-yl]-4-tertpentylbenzenesulfonamide,
N-[(3R)-3-(dimethylamino)-5-methoxy-3,4-dihydro-2H-chromen-8-yl]-3,4,5-trimethoxybenzenesulfonamide,
N-[(3R)-3-(dimethylamino)-5-methoxy-3,4-dihydro-2H-chromen-8-yl]-3-methylbenzenesulfonamide,
N-[(3R)-3-(dimethylamino)-5-methoxy-3,4-dihydro-2H-chromen-8-yl]-2-(trifluoromethoxy)benzenesulfonamide,
4-bromo-N-[(3R)-3-(dimethylamino)-5-methoxy-3,4-dihydro-2H-chromen-8-yl]-2-fluorobenzenesulfonamide,
N-[(3R)-3-(dimethylamino)-5-methoxy-3,4-dihydro-2H-chromen-8-yl]-2-methyl-4-nitrobenzenesulfonamide,
3-chloro-N-[(3R)-3-(dimethylamino)-5-methoxy-3,4-dihydro-2H-chromen-8-yl]benzenesulfonamide,
2-chloro-N-[(3R)-3-(dimethylamino)-5-methoxy-3,4-dihydro-2H-chromen-8-yl]-4-fluorobenzenesulfonamide,
3-chloro-N-[(3R)-3-(dimethylamino)-5-methoxy-3,4-dihydro-2H-chromen-8-yl]-4-fluorobenzenesulfonamide,
2-chloro-N-[(3R)-3-(dimethylamino)-5-methoxy-3,4-dihydro-2H-chromen-8-yl]-4-(trifluoromethyl)benzenesulfonamide,
N-[(3R)-3-(dimethylamino)-5-methoxy-3,4-dihydro-2H-chromen-8-yl]-1-phenylmethanesulfonamide,
N-[(3R)-3-(dimethylamino)-5-methoxy-3,4-dihydro-2H-chromen-8-yl]-2,4-difluorobenzenesulfonamide,
N-[(3R)-3-(dimethylamino)-5-methoxy-3,4-dihydro-2H-chromen-8-yl]-5-fluoro-2-methylbenzenesulfonamide,
N-[(3R)-3-(dimethylamino)-5-methoxy-3,4-dihydro-2H-chromen-8-yl]-4-(trifluoromethoxy)benzenesulfonamide,
2,5-dichloro-N-[(3R)-3-(dimethylamino)-5-methoxy-3,4-dihydro-2H-chromen-8-yl]benzenesulfonamide,
2,4,6-trichloro-N-[(3R)-3-(dimethylamino)-5-methoxy-3,4-dihydro-2H-chromen-8-yl]benzenesulfonamide,
3-bromo-N-[(3R)-3-(dimethylamino)-5-methoxy-3,4-dihydro-2H-chromen-8-yl]benzenesulfonamide,
3-chloro-N-[(3R)-3-(dimethylamino)-5-methoxy-3,4-dihydro-2H-chromen-8-yl]-2-methylbenzenesulfonamide,
N-[(3R)-3-(dimethylamino)-5-methoxy-3,4-dihydro-2H-chromen-8-yl]-2,3,5,6-tetramethylbenzenesulfonamide,
N-[(3R)-3-(dimethylamino)-5-methoxy-3,4-dihydro-2H-chromen-8-yl]-2-fluorobenzenesulfonamide,
N-[(3R)-3-(dimethylamino)-5-methoxy-3,4-dihydro-2H-chromen-8-yl]-4-fluorobenzenesulfonamide,
N-[(3R)-3-(dimethylamino)-5-methoxy-3,4-dihydro-2H-chromen-8-yl]-2-(trifluoromethyl)benzenesulfonamide,
2,5-dichloro-N-[(3R)-3-(dimethylamino)-5-methoxy-3,4-dihydro-2H-chromen-8-yl]thiophene-3-sulfonamide,
N-[(3R)-3-(dimethylamino)-5-methoxy-3,4-dihydro-2H-chromen-8-yl]-3,4-dimethoxybenzenesulfonamide,
N-[(3R)-3-(dimethylamino)-5-methoxy-3,4-dihydro-2H-chromen-8-yl]-2,5-dimethylbenzenesulfonamide,
N-[(3R)-3-(dimethylamino)-5-methoxy-3,4-dihydro-2H-chromen-8-yl]-3-methoxybenzenesulfonamide,
N-[(3R)-3-(dimethylamino)-5-methoxy-3,4-dihydro-2H-chromen-8-yl]-2,5-difluorobenzenesulfonamide,
N-[(3R)-3-(dimethylamino)-5-methoxy-3,4-dihydro-2H-chromen-8-yl]-4-[2-(phenylsulfonyl)ethyl]benzenesulfonamide,
8-chloro-N-[(3R)-3-(dimethylamino)-5-methoxy-3,4-dihydro-2H-chromen-8-yl]naphthalene-2-sulfonamide,
N-[4-({[(3R)-3-(dimethylamino)-5-methoxy-3,4-dihydro-2H-chromen-8-yl]amino}sulfonyl)phenyl]-2,2,2-trifluoroacetamide,
N-[(3R)-3-(dimethylamino)-5-methoxy-3,4-dihydro-2H-chromen-8-yl]-2-(phenylsulfonyl)benzenesulfonamide,
7-bromo-N-[(3R)-3-(dimethylamino)-5-methoxy-3,4-dihydro-2H-chromen-8-yl]naphthalene-1-sulfonamide,
4-(1,3-benzoxazol-2-yl)-N-[(3R)-3-(dimethylamino)-5-methoxy-3,4-dihydro-2H-chromen-8-yl]benzenesulfonamide,
N-[(3R)-3-(dimethylamino)-5-methoxy-3,4-dihydro-2H-chromen-8-yl]-4-methylnaphthalene-1-sulfonamide,
5-chloro-N-[(3R)-3-(dimethylamino)-5-methoxy-3,4-dihydro-2H-chromen-8-yl]naphthalene-2-sulfonamide,
N-[(3R)-3-(dimethylamino)-5-methoxy-3,4-dihydro-2H-chromen-8-yl]-1,2-dimethyl-1H-imidazole-4-sulfonamide,
N-[(3R)-3-(dimethylamino)-5-methoxy-3,4-dihydro-2H-chromen-8-yl]thiophene-3-sulfonamide,
2-chloro-N-[(3R)-3-(dimethylamino)-5-methoxy-3,4-dihydro-2H-chromen-8-yl]-4,5-difluorobenzenesulfonamide,
N-[(3R)-3-(dimethylamino)-5-methoxy-3,4-dihydro-2H-chromen-8-yl]-4-(methylsulfonyl)benzenesulfonamide,
4-chloro-N-[(3R)-3-(dimethylamino)-5-methoxy-3,4-dihydro-2H-chromen-8-yl]-2,5-difluorobenzenesulfonamide,
N-[(3R)-3-(dimethylamino)-5-methoxy-3,4-dihydro-2H-chromen-8-yl]-1,1′-biphenyl-4-sulfonamide,
2-chloro-4-cyano-N-[(3R)-3-(dimethylamino)-5-methoxy-3,4-dihydro-2H-chromen-8-yl]benzenesulfonamide,
3-chloro-N-[(3R)-3-(dimethylamino)-5-methoxy-3,4-dihydro-2H-chromen-8-yl]-4-methylbenzenesulfonamide,
4-bromo-N-[(3R)-3-(dimethylamino)-5-methoxy-3,4-dihydro-2H-chromen-8-yl]-2-methylbenzenesulfonamide,
4-chloro-N-[(3R)-3-(dimethylamino)-5-methoxy-3,4-dihydro-2H-chromen-8-yl]-2,5-dimethylbenzenesulfonamide,
N-[(3R)-3-(dimethylamino)-5-methoxy-3,4-dihydro-2H-chromen-8-yl]-2,3,4-trifluorobenzenesulfonamide,
4-butyl-N-[(3R)-3-(dimethylamino)-5-methoxy-3,4-dihydro-2H-chromen-8-yl]benzenesulfonamide,
1-(3-chlorophenyl)-N-[(3R)-3-(dimethylamino)-5-methoxy-3,4-dihydro-2H-chromen-8-yl]methanesulfonamide,
N-[(3R)-3-(dimethylamino)-5-methoxy-3,4-dihydro-2H-chromen-8-yl]-2,4,5-trifluorobenzenesulfonamide,
methyl 4-({[(3R)-3-(dimethylamino)-5-methoxy-3,4-dihydro-2H-chromen-8-yl]amino}sulfonyl)-2,5-dimethyl-3-furoate,
5-bromo-6-chloro-N-[(3R)-3-(dimethylamino)-5-methoxy-3,4-dihydro-2H-chromen-8-yl]pyridine-3-sulfonamide,
3-chloro-N-[(3R)-3-(dimethylamino)-5-methoxy-3,4-dihydro-2H-chromen-8-yl]-5-fluoro-2-methylbenzenesulfonamide,
4-bromo-N-[(3R)-3-(dimethylamino)-5-methoxy-3,4-dihydro-2H-chromen-8-yl]-2-ethylbenzenesulfonamide,
N-[(3R)-3-(dimethylamino)-5-methoxy-3,4-dihydro-2H-chromen-8-yl]-6-phenoxypyridine-3-sulfonamide,
2,3,4-trichloro-N-[(3R)-3-(dimethylamino)-5-methoxy-3,4-dihydro-2H-chromen-8-yl]benzenesulfonamide,
4-bromo-N-[(3R)-3-(dimethylamino)-5-methoxy-3,4-dihydro-2H-chromen-8-yl]-2,5-difluorobenzenesulfonamide,
N-[(3R)-3-(dimethylamino)-5-methoxy-3,4-dihydro-2H-chromen-8-yl]-1,1′-biphenyl-3-sulfonamide,
N-[(3R)-3-(dimethylamino)-5-methoxy-3,4-dihydro-2H-chromen-8-yl]-1,1′-biphenyl-2-sulfonamide,
N-[(3R)-3-(dimethylamino)-5-methoxy-3,4-dihydro-2H-chromen-8-yl]-1-pyridin-3-ylmethanesulfonamide,
N-[(3R)-3-(dimethylamino)-5-methoxy-3,4-dihydro-2H-chromen-8-yl]-2,2-diphenylethanesulfonamide,
N-[(3R)-3-(dimethylamino)-5-methoxy-3,4-dihydro-2H-chromen-8-yl]-1-benzofuran-2-sulfonamide,
4-chloro-N′-[(3R)-3-(dimethylamino)-5-methoxy-3,4-dihydro-2H-chromen-8-yl]benzene-1,3-disulfonamide,
N-[(3R)-3-(dimethylamino)-5-methoxy-3,4-dihydro-2H-chromen-8-yl]-4-pentylbenzenesulfonamide,
N-[(3R)-3-(dimethylamino)-5-methoxy-3,4-dihydro-2H-chromen-8-yl]-3-(2-methoxyphenoxy)benzenesulfonamide,
N-[(3R)-3-(dimethylamino)-5-methoxy-3,4-dihydro-2H-chromen-8-yl]-4′-methoxy-1,1′-biphenyl-3-sulfonamide,
N-[(3R)-3-(dimethylamino)-5-methoxy-3,4-dihydro-2H-chromen-8-yl]cyclopropanesulfonamide,
1-[3,5-bis(trifluoromethyl)phenyl]-N-[(3R)-3-(dimethylamino)-5-methoxy-3,4-dihydro-2H-chromen-8-yl]methanesulfonamide,
N-[(3R)-3-(dimethylamino)-5-methoxy-3,4-dihydro-2H-chromen-8-yl]-4-fluoronaphthalene-1-sulfonamide,
N-[(3R)-3-(dimethylamino)-5-methoxy-3,4-dihydro-2H-chromen-8-yl]-3,5-difluorobenzenesulfonamide,
N-[(3R)-3-(dimethylamino)-5-methoxy-3,4-dihydro-2H-chromen-8-yl]-3-fluoro-4-methoxybenzenesulfonamide,
N-[(3R)-3-(dimethylamino)-5-methoxy-3,4-dihydro-2H-chromen-8-yl]-5-[2-(methylthio)pyrimidin-4-yl]thiophene-2-sulfonamide,
1-[3-chloro-5-(trifluoromethyl)pyridin-2-yl]-N-[(3R)-3-(dimethylamino)-5-methoxy-3,4-dihydro-2H-chromen-8-yl]-1H-pyrrole-2-sulfonamide,
2,6-dichloro-N-[(3R)-3-(dimethylamino)-5-methoxy-3,4-dihydro-2H-chromen-8-yl]-4-(trifluoromethyl)benzenesulfonamide,
N-[(3R)-3-(dimethylamino)-5-methoxy-3,4-dihydro-2H-chromen-8-yl]-5-[1-methyl-3-(trifluoromethyl)-1H-pyrazol-5-yl]thiophene-2-sulfonamide,
N-[(3R)-3-(dimethylamino)-5-methoxy-3,4-dihydro-2H-chromen-8-yl]-5-[5-(trifluoromethyl)isoxazol-3-yl]thiophene-2-sulfonamide,
N-[(3R)-3-(dimethylamino)-5-methoxy-3,4-dihydro-2H-chromen-8-yl]-4-fluoro-2-(trifluoromethyl)benzenesulfonamide,
N-[(3R)-3-(dimethylamino)-5-methoxy-3,4-dihydro-2H-chromen-8-yl]-4-fluoro-3-(trifluoromethyl)benzenesulfonamide,
N-[(3R)-3-(dimethylamino)-5-methoxy-3,4-dihydro-2H-chromen-8-yl]-2,4,6-trifluorobenzenesulfonamide,
N-[(3R)-3-(dimethylamino)-5-methoxy-3,4-dihydro-2H-chromen-8-yl]-5-isoxazol-5-ylthiophene-2-sulfonamide,
N-[(3R)-3-(dimethylamino)-5-methoxy-3,4-dihydro-2H-chromen-8-yl]-1-(3-nitrophenyl)methanesulfonamide,
N-[(3R)-3-(dimethylamino)-5-methoxy-3,4-dihydro-2H-chromen-8-yl]-2-fluoro-5-(trifluoromethyl)benzenesulfonamide,
N-[(3R)-3-(dimethylamino)-5-methoxy-3,4-dihydro-2H-chromen-8-yl]-5-methyl-2,1,3-benzothiadiazole-4-sulfonamide,
N-[(3R)-3-(dimethylamino)-5-methoxy-3,4-dihydro-2H-chromen-8-yl]-5-fluoro-3-methyl-1-benzothiophene-2-sulfonamide,
2,3-dichloro-N-[(3R)-3-(dimethylamino)-5-methoxy-3,4-dihydro-2H-chromen-8-yl]-4-methoxybenzenesulfonamide,
1-(4-chlorophenyl)-N-[(3R)-3-(dimethylamino)-5-methoxy-3,4-dihydro-2H-chromen-8-yl]methanesulfonamide,
2,3-dichloro-N-[(3R)-3-(dimethylamino)-5-methoxy-3,4-dihydro-2H-chromen-8-yl]benzenesulfonamide,
5-chloro-N-[(3R)-3-(dimethylamino)-5-methoxy-3,4-dihydro-2H-chromen-8-yl]thiophene-2-sulfonamide,
2-chloro-N-[(3R)-3-(dimethylamino)-5-methoxy-3,4-dihydro-2H-chromen-8-yl]-6-methylbenzenesulfonamide,
3,4-dichloro-N-[(3R)-3-(dimethylamino)-5-methoxy-3,4-dihydro-2H-chromen-8-yl]benzenesulfonamide,
3,5-dichloro-N-[(3R)-3-(dimethylamino)-5-methoxy-3,4-dihydro-2H-chromen-8-yl]benzenesulfonamide,
4-(3-chloro-2-cyanophenoxy)-N-[(3R)-3-(dimethylamino)-5-methoxy-3,4-dihydro-2H-chromen-8-yl]benzenesulfonamide,
5-bromo-N-[(3R)-3-(dimethylamino)-5-methoxy-3,4-dihydro-2H-chromen-8-yl]thiophene-2-sulfonamide,
N-[(3R)-3-(dimethylamino)-5-methoxy-3,4-dihydro-2H-chromen-8-yl]-4-isopropylbenzenesulfonamide,
4-bromo-5-chloro-N-[(3R)-3-(dimethylamino)-5-methoxy-3,4-dihydro-2H-chromen-8-yl]thiophene-2-sulfonamide,
5-chloro-N-[(3R)-3-(dimethylamino)-5-methoxy-3,4-dihydro-2H-chromen-8-yl]-2-methoxybenzenesulfonamide,
N-[(3R)-3-(dimethylamino)-5-methoxy-3,4-dihydro-2H-chromen-8-yl]-3-fluorobenzenesulfonamide,
N-[2-chloro-4-({[(3R)-3-(dimethylamino)-5-methoxy-3,4-dihydro-2H-chromen-8-yl]amino}sulfonyl)phenyl]acetamide,
2,4-dichloro-N-[(3R)-3-(dimethylamino)-5-methoxy-3,4-dihydro-2H-chromen-8-yl]-5-methylbenzenesulfonamide,
N-[(3R)-3-(dimethylamino)-5-methoxy-3,4-dihydro-2H-chromen-8-yl]-2-oxo-1,2,3,4-tetrahydroquinoline-6-sulfonamide,
2,4-dichloro-N-[(3R)-3-(dimethylamino)-5-methoxy-3,4-dihydro-2H-chromen-8-yl]-6-methylbenzenesulfonamide,
N-[(3R)-3-(dimethylamino)-5-methoxy-3,4-dihydro-2H-chromen-8-yl]-3,4-difluorobenzenesulfonamide,
N-[(3R)-3-(dimethylamino)-5-methoxy-3,4-dihydro-2H-chromen-8-yl]-2-methylbenzenesulfonamide,
N-[(3R)-3-(dimethylamino)-5-methoxy-3,4-dihydro-2H-chromen-8-yl]-4-iodobenzenesulfonamide,
3-Chloro-N-[(3R)-5-methoxy-3-pyrrolidin-1-yl-3,4-dihydro-2H-chromen-8-yl]-4-methylbenzenesulfonamide, and
5-Chloro-N-[(3R)-5-methoxy-3-pyrrolidin-1-yl-3,4-dihydro-2H-chromen-8-yl]naphthalene-2-sulfonamide,
or salts, solvates or solvated salts thereof.

9. A compound according to claim 1, wherein said compound is selected from
(2S)-5-{[(3-Bromophenyl)sulfonyl]amino}-N,N-dimethyl-1,2,3,4-tetrahydronaphthalen-2-ammonium acetate,
N-[(6S)-4-Bromo-6-(dimethylamino)-5,6,7,8-tetrahydronaphthalen-1-yl]benzenesulfonamide,
N-[(6S)-4-Bromo-6-(dimethylamino)-5,6,7,8-tetrahydronaphthalen-1-yl]-3-chloro-4-fluorobenzenesulfonamide,
4-bromo-N-[(6S)-6-(dimethylamino)-4-methoxy-5,6,7,8-tetrahydronaphthalen-1-yl]-2-ethylbenzenesulfonamide,
5-bromo-6-chloro-N-[(6S)-6-(dimethylamino)-4-methoxy-5,6,7,8-tetrahydronaphthalen-1-yl]pyridine-3-sulfonamide,
N-[(6S)-6-(dimethylamino)-4-methoxy-5,6,7,8-tetrahydronaphthalen-1-yl]-2,3-dihydro-1,4-benzodioxine-6-sulfonamide,
N-[(6S)-6-(dimethylamino)-4-methoxy-5,6,7,8-tetrahydronaphthalen-1-yl]-1,1′-biphenyl-2-sulfonamide,
N-[(6S)-6-(dimethylamino)-4-methoxy-5,6,7,8-tetrahydronaphthalen-1-yl]-1-pyridin-3-ylmethanesulfonamide,
4-chloro-N′-[(6S)-6-(dimethylamino)-4-methoxy-5,6,7,8-tetrahydronaphthalen-1-yl]benzene-1,3-disulfonamide,
5-chloro-N-[(6S)-6-(dimethylamino)-4-methoxy-5,6,7,8-tetrahydronaphthalen-1-yl]naphthalene-1-sulfonamide,
N-[(6S)-6-(dimethylamino)-4-methoxy-5,6,7,8-tetrahydronaphthalen-1-yl]-4-fluoro-3-(trifluoromethyl)benzenesulfonamide,
N-[(6S)-6-(dimethylamino)-4-methoxy-5,6,7,8-tetrahydronaphthalen-1-yl]-5-fluoro-3-methyl-1-benzothiophene-2-sulfonamide,
1-(4-chlorophenyl)-N-[(6S)-6-(dimethylamino)-4-methoxy-5,6,7,8-tetrahydronaphthalen-1-yl]methanesulfonamide,
2-chloro-4-cyano-N-[(6S)-6-(dimethylamino)-4-methoxy-5,6,7,8-tetrahydronaphthalen-1-yl]benzenesulfonamide,
6-chloro-N-[(6S)-6-(dimethylamino)-4-methoxy-5,6,7,8-tetrahydronaphthalen-1-yl]imidazo[2,1-b][1,3]thiazole-5-sulfonamide,
N-[(6S)-6-(dimethylamino)-4-methoxy-5,6,7,8-tetrahydronaphthalen-1-yl]-2-(methylsulfonyl)benzenesulfonamide,
7-chloro-N-[(6S)-6-(dimethylamino)-4-methoxy-5,6,7,8-tetrahydronaphthalen-1-yl]-2,1,3-benzoxadiazole-4-sulfonamide,
4,5-dibromo-N-[(6S)-6-(dimethylamino)-4-methoxy-5,6,7,8-tetrahydronaphthalen-1-yl]thiophene-2-sulfonamide,
5-bromo-N-[(6S)-6-(dimethylamino)-4-methoxy-5,6,7,8-tetrahydronaphthalen-1-yl]-2-methoxybenzenesulfonamide,
N-[(6S)-6-(dimethylamino)-4-methoxy-5,6,7,8-tetrahydronaphthalen-1-yl]-4-phenoxybenzenesulfonamide,
1-acetyl-5-bromo-N-[(6S)-6-(dimethylamino)-4-methoxy-5,6,7,8-tetrahydronaphthalen-1-yl]indoline-6-sulfonamide,
N-[(6S)-6-(dimethylamino)-4-methoxy-5,6,7,8-tetrahydronaphthalen-1-yl]-4-propylbenzenesulfonamide,
4-cyano-N-[(6S)-6-(dimethylamino)-4-methoxy-5,6,7,8-tetrahydronaphthalen-1-yl]benzenesulfonamide,
5-chloro-N-[(6S)-6-(dimethylamino)-4-methoxy-5,6,7,8-tetrahydronaphthalen-1-yl]thiophene-2-sulfonamide,
N-[(6S)-6-(dimethylamino)-4-methoxy-5,6,7,8-tetrahydronaphthalen-1-yl]naphthalene-2-sulfonamide,
3-chloro-N-[(6S)-6-(dimethylamino)-4-methoxy-5,6,7,8-tetrahydronaphthalen-1-yl]-4-methylbenzenesulfonamide,
4-bromo-N-[(6S)-6-(dimethylamino)-4-methoxy-5,6,7,8-tetrahydronaphthalen-1-yl]-2-methylbenzenesulfonamide,
4-chloro-N-[(6S)-6-(dimethylamino)-4-methoxy-5,6,7,8-tetrahydronaphthalen-1-yl]-2,5-dimethylbenzenesulfonamide,
N-[(6S)-6-(dimethylamino)-4-methoxy-5,6,7,8-tetrahydronaphthalen-1-yl]-2,3,4-trifluorobenzenesulfonamide,
1-(3-chlorophenyl)-N-[(6S)-6-(dimethylamino)-4-methoxy-5,6,7,8-tetrahydronaphthalen-1-yl]methanesulfonamide,
N-[(6S)-6-(dimethylamino)-4-methoxy-5,6,7,8-tetrahydronaphthalen-1-yl]-2,4,5-trifluorobenzenesulfonamide,
3-chloro-N-[(6S)-6-(dimethylamino)-4-methoxy-5,6,7,8-tetrahydronaphthalen-1-yl]-5-fluoro-2-methylbenzenesulfonamide,
N-[(6S)-6-(dimethylamino)-4-methoxy-5,6,7,8-tetrahydronaphthalen-1-yl]-6-phenoxypyridine-3-sulfonamide,
4-bromo-N-[(6S)-6-(dimethylamino)-4-methoxy-5,6,7,8-tetrahydronaphthalen-1-yl]-2,5-difluorobenzenesulfonamide,
N-[(6S)-6-(dimethylamino)-4-methoxy-5,6,7,8-tetrahydronaphthalen-1-yl]-1-pyridin-2-ylmethanesulfonamide,
N-[(6S)-6-(dimethylamino)-4-methoxy-5,6,7,8-tetrahydronaphthalen-1-yl]-1,1′-biphenyl-3-sulfonamide,
N-[(6S)-6-(dimethylamino)-4-methoxy-5,6,7,8-tetrahydronaphthalen-1-yl]-1-benzofuran-2-sulfonamide,
4-chloro-N′-[(6S)-6-(dimethylamino)-4-methoxy-5,6,7,8-tetrahydronaphthalen-1-yl]benzene-1,3-disulfonamide,
N-[(6S)-6-(dimethylamino)-4-methoxy-5,6,7,8-tetrahydronaphthalen-1-yl]-3-(2-methoxyphenoxy)benzenesulfonamide,
N-[(6S)-6-(dimethylamino)-4-methoxy-5,6,7,8-tetrahydronaphthalen-1-yl]-4′-methoxy-1,1′-biphenyl-3-sulfonamide,
N-[(6S)-6-(dimethylamino)-4-methoxy-5,6,7,8-tetrahydronaphthalen-1-yl]cyclopropanesulfonamide,
N-[(6S)-6-(dimethylamino)-4-methoxy-5,6,7,8-tetrahydronaphthalen-1-yl]-4-fluoronaphthalene-1-sulfonamide,
N-[(6S)-6-(dimethylamino)-4-methoxy-5,6,7,8-tetrahydronaphthalen-1-yl]-3,5-difluorobenzenesulfonamide,
N-[(6S)-6-(dimethylamino)-4-methoxy-5,6,7,8-tetrahydronaphthalen-1-yl]-3-fluoro-4-methoxybenzenesulfonamide,
1-[3-chloro-5-(trifluoromethyl)pyridin-2-yl]-N-[(6S)-6-(dimethylamino)-4-methoxy-5,6,7,8-tetrahydronaphthalen-1-yl]-1H-pyrrole-2-sulfonamide,
N-[(6S)-6-(dimethylamino)-4-methoxy-5,6,7,8-tetrahydronaphthalen-1-yl]-5-[5-(trifluoromethyl)isoxazol-3-yl]thiophene-2-sulfonamide,
N-[(6S)-6-(dimethylamino)-4-methoxy-5,6,7,8-tetrahydronaphthalen-1-yl]-2,4,6-trifluorobenzenesulfonamide,
N-[(6S)-6-(dimethylamino)-4-methoxy-5,6,7,8-tetrahydronaphthalen-1-yl]-5-isoxazol-5-ylthiophene-2-sulfonamide,
N-[(6S)-6-(dimethylamino)-4-methoxy-5,6,7,8-tetrahydronaphthalen-1-yl]-1-(3-nitrophenyl)methanesulfonamide,
N-[(6S)-6-(dimethylamino)-4-methoxy-5,6,7,8-tetrahydronaphthalen-1-yl]-2-fluoro-5-(trifluoromethyl)benzenesulfonamide,
2,3-dichloro-N-[(6S)-6-(dimethylamino)-4-methoxy-5,6,7,8-tetrahydronaphthalen-1-yl]-4-methoxybenzenesulfonamide,
N-[(6S)-6-(dimethylamino)-4-methoxy-5,6,7,8-tetrahydronaphthalen-1-yl]-4-methylbenzenesulfonamide,
5-(dimethylamino)-N-[(6S)-6-(dimethylamino)-4-methoxy-5,6,7,8-tetrahydronaphthalen-1-yl]naphthalene-1-sulfonamide,
N-[(6S)-6-(dimethylamino)-4-methoxy-5,6,7,8-tetrahydronaphthalen-1-yl]-2-nitrobenzenesulfonamide,
N-[(6S)-6-(dimethylamino)-4-methoxy-5,6,7,8-tetrahydronaphthalen-1-yl]-4-nitrobenzenesulfonamide,
N-[(6S)-6-(dimethylamino)-4-methoxy-5,6,7,8-tetrahydronaphthalen-1-yl]benzenesulfonamide,
N-[(6S)-6-(dimethylamino)-4-methoxy-5,6,7,8-tetrahydronaphthalen-1-yl]-3,4,5-trimethoxybenzenesulfonamide,
N-[(6S)-6-(dimethylamino)-4-methoxy-5,6,7,8-tetrahydronaphthalen-1-yl]-1-phenylmethanesulfonamide,
N-[(6S)-6-(dimethylamino)-4-methoxy-5,6,7,8-tetrahydronaphthalen-1-yl]-4-fluorobenzenesulfonamide,
N-[(6S)-6-(dimethylamino)-4-methoxy-5,6,7,8-tetrahydronaphthalen-1-yl]-4-isopropylbenzenesulfonamide,
N-[(6S)-6-(dimethylamino)-4-methoxy-5,6,7,8-tetrahydronaphthalen-1-yl]-4-iodobenzenesulfonamide,
3-bromo-5-chloro-N-[(6S)-6-(dimethylamino)-4-methoxy-5,6,7,8-tetrahydronaphthalen-1-yl]thiophene-2-sulfonamide,
4-tert-butyl-N-[(6S)-6-(dimethylamino)-4-methoxy-5,6,7,8-tetrahydronaphthalen-1-yl]benzenesulfonamide,
N-[(6S)-6-(dimethylamino)-4-methoxy-5,6,7,8-tetrahydronaphthalen-1-yl]-4-methoxybenzenesulfonamide,
2-chloro-N-[(6S)-6-(dimethylamino)-4-methoxy-5,6,7,8-tetrahydronaphthalen-1-yl]benzenesulfonamide,
N-[4-({[(6S)-6-(dimethylamino)-4-methoxy-5,6,7,8-tetrahydronaphthalen-1-yl]amino}sulfonyl)phenyl]acetamide,
2-bromo-N-[(6S)-6-(dimethylamino)-4-methoxy-5,6,7,8-tetrahydronaphthalen-1-yl]benzenesulfonamide,
N-{[5-({[(6S)-6-(dimethylamino)-4-methoxy-5,6,7,8-tetrahydronaphthalen-1-yl]amino}sulfonyl)thien-2-yl]methyl}benzamide,
N-[(6S)-6-(dimethylamino)-4-methoxy-5,6,7,8-tetrahydronaphthalen-1-yl]-4-(trifluoromethyl)benzenesulfonamide,
N-[(6S)-6-(dimethylamino)-4-methoxy-5,6,7,8-tetrahydronaphthalen-1-yl]-4-ethylbenzenesulfonamide,
2-chloro-N-[(6S)-6-(dimethylamino)-4-methoxy-5,6,7,8-tetrahydronaphthalen-1-yl]-5-(trifluoromethyl)benzenesulfonamide,
N-[(6S)-6-(dimethylamino)-4-methoxy-5,6,7,8-tetrahydronaphthalen-1-yl]-4-methyl-3-nitrobenzenesulfonamide,
N-[(6S)-6-(dimethylamino)-4-methoxy-5,6,7,8-tetrahydronaphthalen-1-yl]naphthalene-1-sulfonamide,
4-chloro-N-[(6S)-6-(dimethylamino)-4-methoxy-5,6,7,8-tetrahydronaphthalen-1-yl]-3-nitrobenzenesulfonamide,
N-[(6S)-6-(dimethylamino)-4-methoxy-5,6,7,8-tetrahydronaphthalen-1-yl]-3-(trifluoromethyl)benzenesulfonamide,
4-chloro-N-[(6S)-6-(dimethylamino)-4-methoxy-5,6,7,8-tetrahydronaphthalen-1-yl]benzenesulfonamide,
2,4-dichloro-N-[(6S)-6-(dimethylamino)-4-methoxy-5,6,7,8-tetrahydronaphthalen-1-yl]benzenesulfonamide,
N-[5-({[(6S)-6-(dimethylamino)-4-methoxy-5,6,7,8-tetrahydronaphthalen-1-yl]amino}sulfonyl)-4-methyl-1,3-thiazol-2-yl]acetamide,
N-[(6S)-6-(dimethylamino)-4-methoxy-5,6,7,8-tetrahydronaphthalen-1-yl]thiophene-2-sulfonamide,
3,5-dichloro-N-[(6S)-6-(dimethylamino)-4-methoxy-5,6,7,8-tetrahydronaphthalen-1-yl]-2-hydroxybenzenesulfonamide,
N-[(6S)-6-(dimethylamino)-4-methoxy-5,6,7,8-tetrahydronaphthalen-1-yl]-3-nitrobenzenesulfonamide,
N-[(6S)-6-(dimethylamino)-4-methoxy-5,6,7,8-tetrahydronaphthalen-1-yl]-2,5-dimethoxybenzenesulfonamide,
N-[(6S)-6-(dimethylamino)-4-methoxy-5,6,7,8-tetrahydronaphthalen-1-yl]-5-(phenylsulfonyl)thiophene-2-sulfonamide,
N-[(6S)-6-(dimethylamino)-4-methoxy-5,6,7,8-tetrahydronaphthalen-1-yl]-5-pyridin-2-ylthiophene-2-sulfonamide,
2-cyano-N-[(6S)-6-(dimethylamino)-4-methoxy-5,6,7,8-tetrahydronaphthalen-1-yl]benzenesulfonamide,
5-chloro-N-[(6S)-6-(dimethylamino)-4-methoxy-5,6,7,8-tetrahydronaphthalen-1-yl]-1,3-dimethyl-1H-pyrazole-4-sulfonamide,
N-[(6S)-6-(dimethylamino)-4-methoxy-5,6,7,8-tetrahydronaphthalen-1-yl]-3,5-dimethylisoxazole-4-sulfonamide,
N-[(6S)-6-(dimethylamino)-4-methoxy-5,6,7,8-tetrahydronaphthalen-1-yl]-1-methyl-1H-imidazole-4-sulfonamide,
N-[(6S)-6-(dimethylamino)-4-methoxy-5,6,7,8-tetrahydronaphthalen-1-yl]-5-isoxazol-3-ylthiophene-2-sulfonamide,
methyl 3-({[(6S)-6-(dimethylamino)-4-methoxy-5,6,7,8-tetrahydronaphthalen-1-yl]amino}sulfonyl)thiophene-2-carboxylate,
2,6-dichloro-N-[(6S)-6-(dimethylamino)-4-methoxy-5,6,7,8-tetrahydronaphthalen-1-yl]benzenesulfonamide,
N-[(6S)-6-(dimethylamino)-4-methoxy-5,6,7,8-tetrahydronaphthalen-1-yl]-2,6-difluorobenzenesulfonamide,
N-[(6S)-6-(dimethylamino)-4-methoxy-5,6,7,8-tetrahydronaphthalen-1-yl]-2-methyl-5-nitrobenzenesulfonamide,
N-[(6S)-6-(dimethylamino)-4-methoxy-5,6,7,8-tetrahydronaphthalen-1-yl]-3-methylbenzenesulfonamide,
4-bromo-N-[(6S)-6-(dimethylamino)-4-methoxy-5,6,7,8-tetrahydronaphthalen-1-yl]-2-fluorobenzenesulfonamide,
N-[3-({[(6S)-6-(dimethylamino)-4-methoxy-5,6,7,8-tetrahydronaphthalen-1-yl]amino}sulfonyl)phenyl]acetamide,
N-[(6S)-6-(dimethylamino)-4-methoxy-5,6,7,8-tetrahydronaphthalen-1-yl]-2-methyl-4-nitrobenzenesulfonamide,
3-chloro-N-[(6S)-6-(dimethylamino)-4-methoxy-5,6,7,8-tetrahydronaphthalen-1-yl]benzenesulfonamide,
2-chloro-N-[(6S)-6-(dimethylamino)-4-methoxy-5,6,7,8-tetrahydronaphthalen-1-yl]-4-fluorobenzenesulfonamide,
3-chloro-N-[(6S)-6-(dimethylamino)-4-methoxy-5,6,7,8-tetrahydronaphthalen-1-yl]-4-fluorobenzenesulfonamide,
2-chloro-N-[(6S)-6-(dimethylamino)-4-methoxy-5,6,7,8-tetrahydronaphthalen-1-yl]-4-(trifluoromethyl)benzenesulfonamide,
N-[(6S)-6-(dimethylamino)-4-methoxy-5,6,7,8-tetrahydronaphthalen-1-yl]-2,4-difluorobenzenesulfonamide,
N-[(6S)-6-(dimethylamino)-4-methoxy-5,6,7,8-tetrahydronaphthalen-1-yl]-5-fluoro-2-methylbenzenesulfonamide,
2,5-dichloro-N-[(6S)-6-(dimethylamino)-4-methoxy-5,6,7,8-tetrahydronaphthalen-1-yl]benzenesulfonamide,
3-bromo-N-[(6S)-6-(dimethylamino)-4-methoxy-5,6,7,8-tetrahydronaphthalen-1-yl]benzenesulfonamide,
3-chloro-N-[(6S)-6-(dimethylamino)-4-methoxy-5,6,7,8-tetrahydronaphthalen-1-yl]-2-methylbenzenesulfonamide,
N-[(6S)-6-(dimethylamino)-4-methoxy-5,6,7,8-tetrahydronaphthalen-1-yl]-2-fluorobenzenesulfonamide,
N-[(6S)-6-(dimethylamino)-4-methoxy-5,6,7,8-tetrahydronaphthalen-1-yl]-2-(trifluoromethyl)benzenesulfonamide,
2,5-dichloro-N-[(6S)-6-(dimethylamino)-4-methoxy-5,6,7,8-tetrahydronaphthalen-1-yl]thiophene-3-sulfonamide,
N-[(6S)-6-(dimethylamino)-4-methoxy-5,6,7,8-tetrahydronaphthalen-1-yl]-3,4-dimethoxybenzenesulfonamide,
2,3-dichloro-N-[(6S)-6-(dimethylamino)-4-methoxy-5,6,7,8-tetrahydronaphthalen-1-yl]benzenesulfonamide,
2-chloro-N-[(6S)-6-(dimethylamino)-4-methoxy-5,6,7,8-tetrahydronaphthalen-1-yl]-6-methylbenzenesulfonamide,
3,4-dichloro-N-[(6S)-6-(dimethylamino)-4-methoxy-5,6,7,8-tetrahydronaphthalen-1-yl]benzenesulfonamide,
3,5-dichloro-N-[(6S)-6-(dimethylamino)-4-methoxy-5,6,7,8-tetrahydronaphthalen-1-yl]benzenesulfonamide,
5-bromo-N-[(6S)-6-(dimethylamino)-4-methoxy-5,6,7,8-tetrahydronaphthalen-1-yl]thiophene-2-sulfonamide,
4-bromo-5-chloro-N-[(6S)-6-(dimethylamino)-4-methoxy-5,6,7,8-tetrahydronaphthalen-1-yl]thiophene-2-sulfonamide,
5-chloro-N-[(6S)-6-(dimethylamino)-4-methoxy-5,6,7,8-tetrahydronaphthalen-1-yl]-2-methoxybenzenesulfonamide,
N-[(6S)-6-(dimethylamino)-4-methoxy-5,6,7,8-tetrahydronaphthalen-1-yl]-3-fluorobenzenesulfonamide,
N-[2-chloro-4-({[(6S)-6-(dimethylamino)-4-methoxy-5,6,7,8-tetrahydronaphthalen-1-yl]amino}sulfonyl)phenyl]acetamide,
N-[(6S)-6-(dimethylamino)-4-methoxy-5,6,7,8-tetrahydronaphthalen-1-yl]-2-oxo-1,2,3,4-tetrahydroquinoline-6-sulfonamide,
N-[(6S)-6-(dimethylamino)-4-methoxy-5,6,7,8-tetrahydronaphthalen-1-yl]-3,4-difluorobenzenesulfonamide,
N-[(6S)-6-(dimethylamino)-4-methoxy-5,6,7,8-tetrahydronaphthalen-1-yl]-2-methylbenzenesulfonamide,
N-[(6S)-6-(dimethylamino)-4-methoxy-5,6,7,8-tetrahydronaphthalen-1-yl]-2,5-dimethylbenzenesulfonamide,
N-[(6S)-6-(dimethylamino)-4-methoxy-5,6,7,8-tetrahydronaphthalen-1-yl]-3-methoxybenzenesulfonamide,
N-[(6S)-6-(dimethylamino)-4-methoxy-5,6,7,8-tetrahydronaphthalen-1-yl]-2,5-difluorobenzenesulfonamide,
4-chloro-N-[(6S)-6-(dimethylamino)-4-methoxy-5,6,7,8-tetrahydronaphthalen-1-yl]naphthalene-1-sulfonamide,
N-[(6S)-6-(dimethylamino)-4-methoxy-5,6,7,8-tetrahydronaphthalen-1-yl]-4-[2-(phenylsulfonyl)ethyl]benzenesulfonamide,
8-chloro-N-[(6S)-6-(dimethylamino)-4-methoxy-5,6,7,8-tetrahydronaphthalen-1-yl]naphthalene-2-sulfonamide,
N-[4-({[(6S)-6-(dimethylamino)-4-methoxy-5,6,7,8-tetrahydronaphthalen-1-yl]amino}sulfonyl)phenyl]-2,2,2-trifluoroacetamide,
N-[(6S)-6-(dimethylamino)-4-methoxy-5,6,7,8-tetrahydronaphthalen-1-yl]-2-(phenylsulfonyl)benzenesulfonamide,
7-bromo-N-[(6S)-6-(dimethylamino)-4-methoxy-5,6,7,8-tetrahydronaphthalen-1-yl]naphthalene-1-sulfonamide,
4-(1,3-benzoxazol-2-yl)-N-[(6S)-6-(dimethylamino)-4-methoxy-5,6,7,8-tetrahydronaphthalen-1-yl]benzenesulfonamide,
N-[(6S)-6-(dimethylamino)-4-methoxy-5,6,7,8-tetrahydronaphthalen-1-yl]-4-methylnaphthalene-1-sulfonamide,
5-chloro-N-[(6S)-6-(dimethylamino)-4-methoxy-5,6,7,8-tetrahydronaphthalen-1-yl]naphthalene-2-sulfonamide,
4′-cyano-N-[(6S)-6-(dimethylamino)-4-methoxy-5,6,7,8-tetrahydronaphthalen-1-yl]-1,1′-biphenyl-2-sulfonamide,
N-[(6S)-6-(dimethylamino)-4-methoxy-5,6,7,8-tetrahydronaphthalen-1-yl]-1,2-dimethyl-1H-imidazole-4-sulfonamide,
N-[(6S)-6-(dimethylamino)-4-methoxy-5,6,7,8-tetrahydronaphthalen-1-yl]thiophene-3-sulfonamide,
2-chloro-N-[(6S)-6-(dimethylamino)-4-methoxy-5,6,7,8-tetrahydronaphthalen-1-yl]-4,5-difluorobenzenesulfonamide,
N-[(6S)-6-(dimethylamino)-4-methoxy-5,6,7,8-tetrahydronaphthalen-1-yl]-4-(methylsulfonyl)benzenesulfonamide,
4-chloro-N-[(6S)-6-(dimethylamino)-4-methoxy-5,6,7,8-tetrahydronaphthalen-1-yl]-2,5-difluorobenzenesulfonamide,
N-[(6S)-6-(dimethylamino)-4-methoxy-5,6,7,8-tetrahydronaphthalen-1-yl]-1,1′-biphenyl-4-sulfonamide,
N-[(6S)-6-(dimethylamino)-4-methoxy-5,6,7,8-tetrahydronaphthalen-1-yl]-2-methoxy-4-methylbenzenesulfonamide,
N-[(6S)-4-methoxy-6-pyrrolidin-1-yl-5,6,7,8-tetrahydronaphthalen-1-yl]pyridine-3-sulfonamide,
3,5-Dichloro-N-[(6S)-4-methoxy-6-pyrrolidin-1-yl-5,6,7,8-tetrahydronaphthalen-1-yl]benzenesulfonamide,
N-[(6S)-4-Methoxy-6-pyrrolidin-1-yl-5,6,7,8-tetrahydronaphthalen-1-yl]quinoline-8-sulfonamide,
N-[(6S)-4-Methoxy-6-pyrrolidin-1-yl-5,6,7,8-tetrahydronaphthalen-1-yl]naphthalene-1-sulfonamide,
4′-Chloro-N-[(6S)-4-methoxy-6-(methylamino)-5,6,7,8-tetrahydronaphthalen-1-yl]biphenyl-2-sulfonamide,
4′-Chloro-N-[(6S)-4-methoxy-6-(methylamino)-5,6,7,8-tetrahydronaphthalen-1-yl]-N-methylbiphenyl-2-sulfonamide,
N-[(6S)-4-Methoxy-6-pyrrolidin-1-yl-5,6,7,8-tetrahydronaphthalen-1-yl]naphthalene-1-sulfonamide,
N-[(6S)-6-(Dimethylamino)-4-methoxy-5,6,7,8-tetrahydronaphthalen-1-yl]quinoline-8-sulfonamide,
4′-Chloro-N-[(6S)-6-(dimethylamino)-4-methoxy-5,6,7,8-tetrahydronaphthalen-1-yl]biphenyl-2-sulfonamide, and
4′-Chloro-N-[(6S)-6-(dimethylamino)-4-methoxy-5,6,7,8-tetrahydronaphthalen-1-yl]-N-methylbiphenyl-2-sulfonamide,
or salts, solvates or solvated salts thereof.

10-12. (canceled)

13. A pharmaceutical composition comprising as active ingredient a therapeutically effective amount of a compound according to claim 1, in association with one or more pharmaceutically acceptable diluents, excipients and/or inert carriers.

14. A method of treating 5HT6 mediated disorders Alzheimer's disease, cognitive impairment associated with schizophrenia, obesity, and/or Parkinson's disease comprising administering a pharmaceutical composition according to claim 11.

15. A method of treating 5HT6 mediated disorders Alzheimer's disease, cognitive impairment associated with schizophrenia, obesity, and/or Parkinson's disease comprising administering to a mammal in need of such treatments a therapeutically effective amount of a compound according to Claim 1.

16. An agent for the prevention or treatment of Alzheimer's disease, cognitive impairment associated with schizophrenia, obesity, and/or Parkinson's disease comprising as active ingredient a compound according to Claim 1.

17. A compound selected from
(3R)-5-methoxy-N³,N³-dimethylchromane-3,8-diamine,
(6S)-4-bromo-N⁶,N⁶-dimethyl-5,6,7,8-tetrahydronaphthalene-1,6-diamine,
(6S)-4-methoxy-N⁶,N⁶-dimethyl-5,6,7,8-tetrahydronaphthalene-1,6-diamine,
(6S)-4-methoxy-6-pyrrolidin-1-yl-5,6,7,8-tetrahydronaphthalen-1-amine, and
N-[(2S)-5-amino-8-methoxy-1,2,3,4-tetrahydronaphthalen-2-yl]-2,2,2-trifluoroacetamide.

18. (canceled)

19. A method of treatment according to claim 13, wherein said mammal is man.

20. A pharmaceutical composition comprising as active ingredient a therapeutically effective amount of a compound according to claim 8, in association with one or more pharmaceutically acceptable diluents, excipients and/or inert carriers.

21. A pharmaceutical composition comprising as active ingredient a therapeutically effective amount of the compound according to claim 9, in association with one or more Pharmaceutically acceptable diluents, excipients and/or inert carriers.

22. A method of treating a 5HT6 mediated disorder, Alzheimer's disease, cognitive impairment associated with schizophrenia, obesity, and/or Parkinson's disease comprising administering to a mammal in need of such treatment a therapeutically effective amount of a compound according to claim 8.

23. A method of treating a 5HT6 mediated disorder, Alzheimer's disease, cognitive impairment associated with schizophrenia, obesity, and/or Parkinson's disease comprising administering to a mammal in need of such treatment a therapeutically effective amount of a compound according to claim 9.
